(19) United States
(12) Patent Application Publication
(10) Pub. No.: US 2006/0121085 A1
(43) Pub. Date: Jun. 8, 2006

(54) Formulations for cell-schedule dependent anticancer agents

(76) Inventors: Stephen L. Warren, Wapchung, NJ (US); Eric Dadey, Fort Collins, CO (US); Mingxing Zhou, Fort Collins, CO (US); Richard L. Dunn, Fort Collins, CO (US)

Correspondence Address: Schwegman, Lundberg, Woessner & Kluth, P. A., P. O. Box 2938, Minneapolis, MN, 55402, US

(21) Appl. No.: 11/222,668

(22) Filed: Sep. 9, 2005

Related U.S. Application Data
(63) Continuation of application No. PCT/US04/07650, filed on Mar. 11, 2004.
(60) Provisional application No. 60/454,100, filed on Mar. 11, 2003.
(60) Provisional application No. 60/505,124, filed on Sep. 22, 2003.

(51) Int. Cl. A61K 31/7072; A61F 2/00; A61K 9/14

(52) U.S. Cl. 424/426; 424/486; 514/49

(57) ABSTRACT

The present invention provides a flowable composition suitable for use as a controlled release implant. The composition includes: (a) a biodegradable, biocompatible thermoplastic polymer that is at least substantially insoluble in aqueous medium, water or body fluid; (b) a cell-cycle dependent biological agent, a schedule-dependent biological agent, a metabolite thereof, a pharmaceutically acceptable salt thereof, or a prodrug thereof; and (c) a biocompatible organic liquid, at standard temperature and pressure, in which the thermoplastic polymer is soluble. The present invention also provides a method of treating cancer in a mammal. The present invention also provides a method of blocking, impeding, or otherwise interfering with cell cycle progression at the G1-phase, G1/S interphase, S-phase, G2/M interface or M-phase of the cell cycle in a mammal. The methods includes administering to a mammal an effective amount of a flowable composition of the present invention.

### RELATED APPLICATIONS

This patent application is a U.S. National Stage Filing Under 35 U.S.C. 111(a) of PCT/US04/07650, filed Mar. 11, 2004 and published on Sep. 23, 2004 as WO 2004/081196 A2, which claims the benefit of priority, under 35 U.S.C. §119(e), to U.S. Provisional Patent Application Ser. No. 60/454,100, filed on Mar. 11, 2003, and to U.S. Provisional Patent Application Ser. No. 60/505,124, filed on Sep. 22, 2003, which applications are herein incorporated by reference in its entirety.

### BACKGROUND OF THE INVENTION

Cancer is a general term frequently used to indicate any of the various types of malignant neoplasms (i.e., abnormal tissue that grows by uncontrolled cellular proliferation), most of which invade surrounding tissue, may metastasize to several sites, are likely to recur after attempted removal, and causes death unless adequately treated. Stedman's Medical Dictionary, 25th Edition Illustrated, Williams & Wilkins, 1990. Approximately 1.2 million Americans are diagnosed with cancer each year, 8,000 of which are children. In addition, 500,000 Americans die from cancer each year in the United States alone. Specifically, lung and prostate cancer are the top cancer killers for men while lung and breast cancer are the top cancer killers for women. It is estimated that cancer-related costs account for about 10 percent of the total amount spent on disease treatment in the United States. CNN Cancer Facts, http://www.cnn.com/HEALTH/9511/conquer_cancer/facts/index.html, page 2 of 2, Jul. 18, 1999.

Although a variety of approaches to cancer therapy (e.g., surgical resection, radiation therapy, and chemotherapy) have been available and commonly used for many years, cancer remains one of the leading causes of death in the world. This is due in part to the therapies themselves causing significant toxic side-effects as well as the re-emergence of the deadly disease. Though effective in some kinds of cancers, the use of systemic chemotherapy has had minor success in the treatment of cancer of the colon-rectum, esophagus, liver, pancreas, kidney and melanoma. A major problem with systemic chemotherapy for the treatment of these types of cancer is that the systemic doses required to achieve control of tumor growth frequently result in unacceptable systemic toxicity.

The toxicity associated with conventional cancer chemotherapy is due primarily to a lack of specificity of the chemotherapeutic agent. Unfortunately, conventional cytotoxic anti-cancer drugs by themselves typically do not distinguish between malignant and normal cells. As a result, anti-cancer drugs are absorbed by both cell types. Thus, conventional chemotherapeutic agents not only destroy diseased cells, but also destroy normal, healthy cells. To overcome this limitation, therapeutic strategies that increase the specificity, increase the efficacy, as well as reduce the toxicity of anti-cancer drugs are being explored. One such strategy that is being aggressively pursued is drug targeting.

An objective of drug targeting is to deliver drugs to a specific site of action through a carrier system. Such targeting achieves at least two major aims of drug delivery. The first is to deliver the maximum dose of therapeutic agent to diseased cells. The second is the avoidance of uptake by normal, healthy cells. Thus, targeted drug delivery systems result in enhancing drug accumulation in tumors while decreasing exposure to susceptible healthy tissues. As such, the efficacy is increased while the toxicity is decreased.

Several references describe flowable compositions suitable for use as a controlled release implant, sustained release delivery systems for use as biodegradable and bioerodible implants; wherein the flowable compositions and sustained release delivery systems include: (a) a biodegradable, biocompatible polymer; (b) a biological agent; and (c) a biocompatible organic liquid; and wherein the resulting implants that are formed in situ include: (a) a biodegradable, biocompatible polymer and (b) a biological agent. See, e.g., U.S. Pat. Nos. 6,565,874; 6,528,080; RE37,950; 6,461,631; 6,395,293; 6,355,657; 6,261,583; 6,143,314; 5,990,194; 5,945,115; 5,792,469; 5,780,044; 5,759,563; 5,744,153; 5,739,176; 5,736,152; 5,733,950; 5,702,716; 5,681,873; 5,599,552; 5,487,897; 5,340,849; 5,324,519; 5,278,202; and 5,278,201. These references do dot describe such articles wherein the biological agent is a cell-cycle dependent biological agent, a schedule-dependent biological agent, a metabolite thereof, a pharmaceutically acceptable salt thereof, or a prodrug thereof. Additionally, these references do dot describe such articles that employ a chemotherapeutic agent that blocks, impedes, or otherwise interferes with cell cycle progression at the G1-phase, G1/S interphase, S-phase, G2/M interface or M-phase of the cell cycle. These references do dot describe such articles that employ a chemotherapeutic agent that has improved specificity (i.e., localize in tumor cells in high concentration compared to normal cells). These references also do not describe such articles that employ a chemotherapeutic agent to be administered in an amount (e.g., dosage) that is significantly lower that the recommended amount.

As such, there is currently a need for chemotherapeutic agents that have improved specificity (i.e., localize in tumor cells in high concentration compared to normal cells), or efficacy, and for chemotherapeutic agents which can selectively target cancer cells.

### SUMMARY OF THE INVENTION

The present invention provides an article of manufacture that includes, as a chemotherapeutic agent, a cell-cycle dependent biological agent, a schedule-dependent biological agent, a metabolite thereof, a pharmaceutically acceptable salt thereof, or a prodrug thereof. Such a chemotherapeutic agent can effectively block, impede, or otherwise interfere with cell cycle progression at the G1-phase, G1/S interphase, S-phase, G2/M interface or M-phase of the cell cycle. This class of chemotherapeutic agents, present in the article of manufacture, has an improved specificity (i.e., will localize in or near tumor cells in high concentration, compared to normal cells). The article of manufacture will include and deliver the chemotherapeutic agent in an amount (e.g., dosage) that can be significantly lower than the recommended amount. This will not only be less expensive that current oncological treatments, but will lessen or diminish the side effects associated with the current administration of these chemotherapeutic agents.

With the administration of the flowable composition of the present invention, local activation of a cell-cycle dependent biological agent or schedule-dependent biological agent (e.g., 125-IUDR) can be achieved, by the activation of a prodrug to the parent drug. Additionally, by employing a prodrug in a suitable flowable composition, prolonged release kinetics can be achieved, as well as an enhanced therapeutic index. This is so because upon administration, the prodrug is sequestered in the depot wherein little or no degradation (e.g., hydrolysis) of the prodrug is encountered, and maximum retention of the prodrug is achieved due to hydrophobicity. The limited biodistribution (i.e., a high local concentration, a low systemic concentration and a rapid hepatic detoxification) provides an acceptable therapeutic index for these toxic chemotherapeutic agents. Bioerosion of the implant exposes the prodrug to aqueous milieu at the tissue interface of the depot. The prodrug degrades (e.g., hydrolyzes), thereby activating it (i.e., converting the prodrug to the parent drug). Any prodrug that escapes into the bloodstream will likely be inactivated by dehalogenation.

Both spatial and temporal requirements are critical for treating forms of cancer that operate via a cell cycle progression at the G1-phase, G1/S interphase, S-phase, G2/M interface or M-phase of the cell cycle. Both the temporal and spatial requirement are achieved with the controlled release implant of the present invention, since the implant will preferably be located in the tumor or tumor margin for days or weeks and since the implant releases a cell-cycle dependent biological agent, schedule-dependent biological agent, metabolite thereof, pharmaceutically acceptable salt thereof, or prodrug thereof.

The present invention provides a flowable composition suitable for use as a controlled release implant. The composition includes: (a) a biodegradable, biocompatible thermoplastic polymer that is at least substantially insoluble in aqueous medium, water or body fluid; (b) a cell-cycle dependent biological agent, a schedule-dependent biological agent, a metabolite thereof, a pharmaceutically acceptable salt thereof, or a prodrug thereof; and (c) a biocompatible organic liquid (e.g., at standard temperature and pressure), in which the thermoplastic polymer is soluble.

The present invention also provides a method of treating cancer in a mammal. The method includes administering to a mammal in need of such treatment an effective amount of a flowable composition of the present invention.

The present invention also provides a method of blocking, impeding, or otherwise interfering with cell cycle progression at the G1-phase, G1/S interphase, S-phase, G2/M interface or M-phase of the cell cycle in a mammal. The method includes administering to a mammal in need of such blocking, impeding, or interfering an effective amount of a flowable composition of the present invention.

The present invention also provides an implant that includes: (a) a biodegradable, biocompatible thermoplastic polymer that is at least substantially insoluble in aqueous medium, water or body fluid; (b) a cell-cycle dependent biological agent, a schedule-dependent biological agent, a metabolite thereof, a pharmaceutically acceptable salt thereof, or a prodrug thereof; and (c) a biocompatible organic liquid at standard temperature and pressure, in which the thermoplastic polymer is soluble; wherein the implant has a solid or gelatinous microporous matrix, the matrix being a core surrounded by a skin and wherein the implant is surrounded by body tissue.

The present invention also provides an implant that includes: (a) a biodegradable, biocompatible thermoplastic polymer that is at least substantially insoluble in aqueous medium, water or body fluid; and (b) a cell-cycle dependent biological agent, a schedule-dependent biological agent, a metabolite thereof, a pharmaceutically acceptable salt thereof, or a prodrug thereof; wherein the implant has a solid or gelatinous microporous matrix, the matrix being a core surrounded by a skin and wherein the implant is surrounded by body tissue.

The present invention also provides a method of forming an implant in situ within a living body. The method includes: (a) injecting a flowable composition within the body of a patient, the composition includes: (i) a biodegradable, biocompatible thermoplastic polymer that is at least substantially insoluble in aqueous medium, water or body fluid; (ii) a cell-cycle dependent biological agent, a schedule-dependent biological agent, a metabolite thereof, a pharmaceutically acceptable salt thereof, or a prodrug thereof; and (iii) a biocompatible organic liquid at standard temperature and pressure, in which the thermoplastic polymer is soluble; and (b) allowing the biocompatible organic liquid to dissipate to produce a solid biodegradable implant.

The present invention also provides a pharmaceutical kit suitable for in situ formation of a biodegradable implant in a body. The kit includes: (a) a first container comprising a flowable composition, the composition includes: (i) a biodegradable, biocompatible thermoplastic polymer that is at least substantially insoluble in aqueous medium, water or body fluid; and (ii) a biocompatible organic liquid at standard temperature and pressure, in which the thermoplastic polymer is soluble; and (b) a second container comprising a cell-cycle dependent biological agent, a schedule-dependent biological agent, a metabolite thereof, a pharmaceutically acceptable salt thereof, or a prodrug thereof.

The present invention also provides a flowable composition of the present invention for use in medical therapy or diagnosis.

The present invention also provides the use of a flowable composition of the present invention for the manufacture of a medicament for treating cancer.

### DETAILED DESCRIPTION OF THE INVENTION

The present invention is directed to a flowable composition suitable for use as a controlled release implant. The composition includes: (a) a biodegradable, biocompatible thermoplastic polymer that is at least substantially insoluble in aqueous medium, water or body fluid; (b) a cell-cycle dependent biological agent, a schedule-dependent biological agent, a metabolite thereof, a pharmaceutically acceptable salt thereof, or a prodrug thereof; and (c) a biocompatible organic liquid, at standard temperature and pressure, in which the thermoplastic polymer is soluble. The thermoplastic polymer is at least substantially, preferably essentially completely soluble, in the organic solvent and is at least substantially, preferably completely insoluble in aqueous medium, body fluid and water. The organic solvent is at least slightly soluble in water, preferably moderately soluble in water, and especially preferably substantially soluble in water. The flowable composition is pharmaceutically suitable for injection into a body wherein it will form a pharmaceutically acceptable, solid matrix, which typically is a single body implant or drug delivery system. The implant will release the cell-cycle dependent biological agent, schedule-dependent biological agent, metabolite thereof, pharmaceutically acceptable salt thereof, or prodrug thereof, at a controlled rate. The rate of release may be altered to be faster or slower by inclusion of a rate-modifying agent.

It is appreciated that those of skill in the art underatand that the terms “soluble” and “insoluble” are relative terms. For example, a substance that has a solubility, in water, of about 1×10⁻⁴⁵ mg/L is relativelt insoluble in water. It none-the-less, has some (i.e., discrete and finite) solubility in water. It is because of this impresice terminology that Applicant employs the terms “solubility ranging from completely insoluble in any proportion to completely soluble in all proportions,” “at least partially water-soluble,” and “completely water-soluble” to describe the organic solvent/liquid.

It is also appreciated that those of skill in the art understand that the solubility of an organic solvent/liquid in boldily fluid can vary, e.g., on the specified bodily fluid and with the specified individual. Since Applicant is unaware of any universally accepted parameters to define an organic liquid/solvent in terms of its solubility in bodily fluids, Applicant has described the organic liquid/solvent in terms of its solubility in water. As such, when reference is made to the solubility of an organic liquid/solvent in water, it is appreciated that those of skill in the art understand that this is to give guidance and direction to an organic liquid/solvent with an equivalent solubility in bolidy fluids. This is so even though it is understood that not all organic liquids/solvents have the same solubility in water than they do in bodily fluids.

The term ester linkage refers to —OC(═O)— or —C(═O)O—; the term thioester linkage refers to —SC(═O)— or —C(═O)S—; the term amide linkage refers to —N(R)C(═O)— or —C(═O)N(R)—, the term phosphoric acid ester refers to —OP(═O)₂O—; the term sulphonic acid ester refers to —SO₂O— or —OSO₂—, wherein each R is a suitable organic radical, such as, for example, hydrogen, (C₁-C₂₀)alkyl, (C₃-C₆)cycloalkyl, (C₃-C₆)cycloalkyl(C₁-C₂₀)alkyl, aryl, heteroaryl, aryl(C₁-C₂₀)alkyl, or heteroaryl(C₁-C₂₀)alkyl.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C1-C6)alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York, John Wiley & sons, Inc., and references cited therein).

The term “peptide” describes a sequence of 2 to 35 amino acids (e.g. as defined hereinabove) or peptidyl residues. The sequence may be linear or cyclic. For example, a cyclic peptide can be prepared or may result from the formation of disulfide bridges between two cysteine residues in a sequence. Preferably a peptide comprises 3 to 20, or 5 to 15 amino acids. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right.

The term “saccharide” refers to any sugar or other carbohydrate, especially a simple sugar or carbohydrate. Saccharides are an essential structural component of living cells and source of energy for animals. The term includes simple sugars with small molecules as well as macromolecular substances. Saccharides are classified according to the number of monosaccharide groups they contain.

The term “polysaccharide” refers to a type of carbohydrate that contains sugar molecules that are linked together chemically, i.e., through a glycosidic linkage. The term refers to any of a class of carbohydrates whose are carbohydrates that are made up of chains of simple sugars. Polysaccharides are polymers composed of multiple units of monosaccharide (simple sugar).

The term “fatty acid” refers to a class of aliphatic monocarboxylic acids that form part of a lipid molecule and can be derived from fat by hydrolysis. The term refers to any of many long lipid-carboxylic acid chains found in fats, oils, and as a component of phospholipids and glycolipids in animal cell membranes.

The term “polyalcohol” refers to a hydrocarbon that includes one or more (e.g., 2, 3, 4, or 5) hydroxyl groups.

The term “carbohydrate” refers to an essential structural component of living cells and source of energy for animals; includes simple sugars with small molecules as well as macromolecular substances; are classified according to the number of monosaccharide groups they contain. The term refers to one of a group of compounds including the sugars, starches, and gums, which contain six (or some multiple of six) carbon atoms, united with a variable number of hydrogen and oxygen atoms, but with the two latter always in proportion as to form water; as dextrose, {C₆H₁₂O₆}. The term refers to a compound or molecule that is composed of carbon, oxygen and hydrogen in the ratio of 2H:1C:1O. Carbohydrates can be simple sugars such as sucrose and fructose or complex polysaccharide polymers such as chitin.

As used herein, “starch” refers to the complex polysaccharides present in plants, consisting of α-(1,4)-D-glucose repeating subunits and α-(1,6)-glucosidic linkages.

As used herein, “dextrin” refers to a polymer of glucose with intermediate chain length produced by partial degradation of starch by heat, acid, enzyme, or a combination thereof.

As used herein, “maltodextrin” or “glucose polymer” refers to non-sweet, nutritive saccharide polymer that consists of D-glucose units linked primarily by α,-1,4 bonds and that has a DE (dextrose equivalent) of less than 20. See, e.g., The United States Food and Drug Administration (21 C.F.R. paragraph 184.1444). Maltodextrins are partially hydrolyzed starch products. Starch hydrolysis products are commonly characterized by their degree of hydrolysis, expressed as dextrose equivalent (DE), which is the percentage of reducing sugar calculated as dextrose on dry-weight basis.

As used herein, “cyclodextrins” refers to a group of naturally occurring clathrates and products by the action of Bacillus macerans amylase on starch, e.g., α-, β-, and γ-cyclodextrins.

Flowable Composition

According to the present invention, a flowable composition is provided in which a biocompatible, biodegradable, thermoplastic polymer and a cell-cycle dependent biological agent, a schedule-dependent biological agent, a metabolite thereof, a pharmaceutically acceptable salt thereof, or a prodrug thereof are dissolved or dispersed in a biocompatible organic solvent.

Upon contact with an aqueous medium, body fluid or water, the flowable composition solidifies to form an implant or implantable article. The implants and implantable articles that are formed from the flowable polymer compositions of the present invention are used for controlled drug release. The cell-cycle dependent biological agent, schedule-dependent biological agent, metabolite thereof, pharmaceutically acceptable salt thereof, or prodrug thereof is contained within the solidified polymer matrix when the flowable composition undergoes its transformation to an implant or implantable article. When the implant is present within a body, the cell-cycle dependent biological agent, schedule-dependent biological agent, metabolite thereof, pharmaceutically acceptable salt thereof, or prodrug thereof is released in a sustained manner through diffusion through the polymer matrix, by direct dissolution at the implant surfaces and by degradation and erosion of the thermoplastic polymer.

Polymer

The biocompatible, biodegradable, thermoplastic polymers used according to the invention can be made from a variety of monomers which form polymer chains or monomeric units joined together by linking groups. These include polymers with polymer chains or backbones containing such linking groups as ester, amide, urethane, anhydride, carbonate, urea, esteramide, acetal, ketal, and orthocarbonate groups as well as any other organic functional group that can be hydrolyzed by enzymatic or hydrolytic reaction (i.e., is biodegradable by this hydrolytic action). These polymers are usually formed by reaction of starting monomers containing the reactant groups that will form these backbone linking groups. For example, alcohols and carboxylic acids will form ester linking groups. Isocyanates and amines or alcohols will respectively form urea or urethane linking groups.

According to the present invention, some fraction of one of these starting monomers will be at least trifunctional, and preferably multifunctional. This multifunctional character provides at least some branching of the resulting polymer chain. For example, when the polymer chosen contains ester linking groups along its polymer backbone, the starting monomers normally will be hydroxycarboxylic acids, cyclic dimmers of hydroxycarboxylic acids, cyclic trimers of hydroxycarboxylic acids, diols or dicarboxylic acids. The polymers of the present invention are obtained by inclusion of some fraction of a starting monomer that is at least multifunctional. In addition, the polymers of the present invention may incorporate more than one multifunctional unit per polymer molecule, and typically many multifunctional units depending on the stoichiometry of the polymerization reaction. Preferably, the polymers of the present invention incorporate at least one multifunctional unit per polymer molecule. A so-called star or branched polymer is formed when one multifunctional unit is incorporated in each polymer molecule. The biodegradable, biocompatible thermoplastic polymer of the present invention can be a linear polymer; or the biodegradable, biocompatible thermoplastic polymer of the present invention can be a branched polymer.

For example, for the ester linking group polymer described above, a dihydroxycarboxylic acid would be included with the first kind of starting monomer, or a triol and/or a tricarboxylic acid would be included with the second kind of starting monomer. Similarly, a triol, quatraol, pentaol, or hexaol such as sorbitol or glucose can be included with the first kind of starting monomer. The same rationale would apply to polyamides. A triamine and/or triacid would be included with starting monomers of a diamine and dicarboxylic acid. An amino dicarboxylic acid, diamino carboxylic acid or a triamine would be included with the second kind of starting monomer, amino acid. Any aliphatic, aromatic or arylalkyl starting monomer having the specified functional groups can be used according to the invention to make the branched thermoplastic polymers of the invention, provided that the polymers and their degradation products are biocompatible. The biocompatiblity specifications of such starting monomers are known in the art.

In particular, the monomers used to make the biocompatible thermoplastic branched polymers of the present invention will produce polymers or copolymers that are biocompatible and biodegradable. Examples of biocompatible, biodegradable polymers suitable for use as the biocompatible thermoplastic branched polymers of the present invention include polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), and copolymers, terpolymers, or combinations or mixtures of the above materials.

The polymer composition of the invention can also include polymer blends of the polymers of the present invention with other biocompatible polymers, so long as they do not interfere undesirably with the biodegradable characteristics of the composition. Blends of the polymer of the invention with such other polymers may offer even greater flexibility in designing the precise release profile desired for targeted drug delivery or the precise rate of biodegradability desired for structural implants such as for orthopedic applications.

The preferred biocompatible thermoplastic polymers or copolymers of the present invention are those which have a lower degree of crystallization and are more hydrophobic. These polymers and copolymers are more soluble in the biocompatible organic solvents than highly crystalline polymers such as polyglycolide or chitin, which have a high degree of hydrogen-bonding. Preferred materials with the desired solubility parameters are branched polylactides, polycaprolactones, and copolymers of these with glycolide in, which there are more amorphous regions to enhance solubility. Generally, the biocompatible, biodegradable thermoplastic polymer is substantially soluble in the organic solvents so that up to 50-60 wt % solids can be made. Preferably, the polymers used according to the invention are essentially completely soluble in the organic solvent so that mixtures up to 85-98 wt % solids can be made. The polymers also are at least substantially insoluble in water so that less than 0.1 g of polymer per mL of water will dissolve or disperse in water. Preferably, the polymers used according to the invention are essentially completely insoluble in water so that less than 0.001 g of polymer per mL of water will dissolve or disperse in water. At this preferred level, the flowable composition with a completely water miscible solvent will almost immediately transform to the solid polymer.

Solvent/Liquid

Liquids suitable for use in the flowable composition are biocompatible and are at least slightly soluble in aqueous medium, body fluid, or water. The organic liquid preferably is at least moderately soluble, more preferably very soluble, and most preferably soluble at all concentrations in aqueous medium, body fluid, or water. An organic liquid that is at least slightly soluble in aqueous or body fluid will allow water to permeate into the polymer solution over a period of time ranging from seconds to weeks and cause it to coagulate or solidify. The slightly soluble liquids will slowly diffuse from the flowable composition and typically will enable the transformation over a period of days to weeks, e.g. about a day to several weeks. The moderately soluble to very soluble organic liquids will diffuse from the flowable composition over a period of minutes to days so that the transformation will occur rapidly but with sufficient leisure to allow its manipulation as a pliable implant after its placement. The highly soluble organic liquids will diffuse from the flowable composition over a period of seconds to hours so that the transformation will occur almost immediately. The organic liquid preferably is a polar aprotic or polar protic organic solvent. Preferably, the organic solvent has a molecular weight in the range of about 30 to about 1000.

Although it is not meant as a limitation of the invention, it is believed that the transition of the flowable composition to a solid is the result of the dissipation of the organic liquid from the flowable composition into the surrounding aqueous medium or body fluid and the infusion of water from the surrounding aqueous medium or body fluid into the organic liquid within the flowable composition. It is believed that during this transition, the thermoplastic polymer and organic liquid within the flowable composition partition into regions rich and poor in polymer. The regions poor in polymer become infused with water and yield the porous nature of the resulting solid structure.

Examples of biocompatible organic liquids that may be used to form the flowable compositions of the present invention include aliphatic, aryl, and arylalkyl linear, cyclic and branched organic compounds that are liquid or at least flowable at ambient and physiological temperature and contain such functional groups as alcohols, ketones, ethers, amides, esters, carbonates, sulfoxides, sulfones, and any other functional group that is compatible with living tissue.

Preferred biocompatible organic liquids that are at least slightly soluble in aqueous or body fluid include N-methyl-2-pyrrolidone, 2-pyrrolidone; C₁ to C₁₅ alcohols, diols, triols and tetraols such as ethanol, glycerine, propylene glycol, butanol; C₃ to C₁₅ alkyl ketones such as acetone, diethyl ketone and methyl ethyl ketone; C₃ to C₁₅ esters such as methyl acetate, ethyl acetate, ethyl lactate; C₁ to C₁₅ amides such as dimethylformamide, dimethylacetamide and caprolactam; C₃ to C₂₀ ethers such as tetrahydrofuran, or solketal; tweens, triacetin, propylene carbonate, decylmethylsulfoxide, dimethyl sulfoxide, oleic acid, and 1-dodecylazacycloheptan-2-one. Other preferred organic liquids are benzyl alcohol, benzyl benzoate, dipropylene glycol, tributyrin, ethyl oleate, glycerin, glycofural, isopropyl myristate, isopropyl palmitate, oleic acid, polyethylene glycol, propylene carbonate, and triethyl citrate. The most preferred solvents are N-methyl-2-pyrrolidone, 2-pyrrolidone, dimethyl sulfoxide, triacetin, and propylene carbonate because of their solvating ability and their compatibility.

The solubility of the biodegradable thermoplastic polymers in the various organic liquids will differ depending upon their crystallinity, their hydrophilicity, hydrogen-bonding, and molecular weight. Lower molecular-weight polymers will normally dissolve more readily in the organic liquids than high-molecular-weight polymers. As a result, the concentration of a polymer dissolved in the various organic liquids will differ depending upon type of polymer and its molecular weight. Moreover, the higher molecular-weight polymers will tend to give higher solution viscosities than the low-molecular-weight materials.

Generally, the concentration of the polymer in the organic liquid according to the invention will range from about 0.01 g per ml of organic liquid to a saturated concentration. Typically, the saturated concentration will be in the range of 80 to 95 wt % solids or 4 to almost 5 gm per ml of organic liquid, assuming that the solvent weighs approximately 1 gm per ml.

For polymers that tend to coagulate slowly, a solvent mixture can be used to increase the coagulation rate. In essence, one liquid component of the solvent mixture is a good solvent for the polymer, and the other liquid component of the solvent mixture is a poorer solvent or a non-solvent. The two liquids are mixed at a ratio such that the polymer is still soluble but precipitates with the slightest increase in the amount of non-solvent, such as water in a physiological environment. By necessity, the solvent system must be miscible with both the polymer and water. An example of such a binary solvent system is the use of N-methyl pyrrolidone and ethanol. The addition of ethanol to the NMP/polymer solution increases its coagulation rate.

The pliability of the composition can be substantially maintained throughout its life as an implant if a certain subgroup of the organic liquid of the composition is used. Such organic liquid also can act as a plasticizer for the thermoplastic polymer and at least in part may remain in the composition rather than dispersing into body fluid, especially when the organic liquid has low water solubility. Such an organic liquid having these low water solubility and plasticizing properties may be included in the composition in addition to the organic liquid that is highly water soluble. In the latter situation, the first organic liquid preferably will rapidly disperse into the body fluid.

Organic liquids of low water solubility, i.e. those forming aqueous solutions of no more than 5% by weight in water can also be used as the organic liquid of the implant composition. Such organic liquids can also act as plasticizers for the thermoplastic polymer. When the organic liquid has these properties, it is a member of a subgroup of organic solvents termed “plasticizer organic liquids” herein. The plasticizer organic liquid influences the pliablity and moldability of the implant composition such that it is rendered more comfortable to the patient when implanted. Moreover, the plasticizer organic liquid has an effect upon the rate of sustained release of the biologically active agent such that the rate can be increased or decreased according to the character of the plasticizer organic liquid incorporated into the implant composition. Although the organic liquid of low water solubility and plasticizing ability can be used alone as the organic liquid of the implant composition, it is preferable to use it in combination as follows. When a high water solubility organic liquid is chosen for primary use in the implant composition, the plasticizer effect can be achieved by use of a second organic liquid having a low water solubility and a plasticizing ability. In this instance, the second organic liquid is a member of the organic liquid subgroup and at least in part will remain in the implant composition for a sustained period. In general, the organic liquid acting as a plasticizer is believed to facilitate molecular movement within the solid thermoplastic matrix. The plasticizing capability enables polymer molecules of the matrix to move relative to each other so that pliability and easy moldability are provided. The plasticizing capability also enables easy movement of the bioactive agent so that in some situations, the rate of sustained release is either positively or negatively affected.

High Water Solubility Organic Liquids/Solvents

A highly water soluble organic liquid can be generally used in the implant composition and especially when pliability will not be an issue after implantation of the implant composition. Use of the highly water soluble organic liquid will produce an implant having the physical characteristics of and implant made through direct insertion of the flowable composition. Such implants and the precursor flowable compositions are described, for example in U.S. Pat. Nos. 4,938,763 and 5,278,201, the disclosures of which are incorporated herein by reference.

Useful, highly water soluble organic liquids include, for example, substituted heterocyclic compounds such as N-methyl-2-pyrrolidone (NMP) and 2-pyrrolidone; C₂ to C₁₀ alkanoic acids such as acetic acid and lactic acid, esters of hydroxy acids such as methyl lactate, ethyl lactate, alkyl citrate and the like; monoesters of polycarboxylic acids such as monomethyl succinate acid, monomethyl citric acid and the like; ether alcohols such as glycofurol, glycerol formal, isopropylidene glycol, 2,2-dimethyl-1,3-dioxolone-4-methanol; Solketal; dialkylamides such as dimethylformamide, dimethylacetamide; dimethylsulfoxide (DMSO) and dimethylsulfone; lactones such as epsilon, caprolactone and butyrolactone; cyclic alkyl amides such as caprolactam; and mixtures and combinations thereof. Preferred organic liquids include N-methyl-2-pyrrolidone, 2-pyrrolidone, dimethylsulfoxide, ethyl lactate, glycofurol, glycerol formal, and isopropylidene glycol.

Low Water Solubility Organic Liquids/Solvents

As described above, a low water solubility organic liquid may also be used in the implant composition. Preferably, a low water solubility liquid is used when it is desirable to have an implant that remains pliable and is extrudable. Also, the release rate of the biologically active agent can be affected under some circumstances through the use of an organic liquid of low water solubility. Typically such circumstances involve retention of the organic liquid within the implant product and its function as a plasticizer.

Examples of low water soluble organic liquids include esters of carbonic acid and aryl alcohols such as benzyl benzoate; C₄ to C₁₀ alkyl alcohols; C₁ to C₆ alkyl C₂ to C₆ alkanoates; esters of carbonic acid and alkyl alcohols such as propylene carbonate, ethylene carbonate and dimethyl carbonate, alkyl esters of mono-, di-, and tricarboxylic acids, such as 2-ethyoxyethyl acetate, ethyl acetate, methyl acetate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate; alkyl ketones such as methyl ethyl ketone; as well as other carbonyl, ether, carboxylic ester, amide and hydroxy containing liquid organic compounds having some solubility in water. Propylene carbonate, ethyl acetate, triethyl citrate, isopropyl myristate, and glyceryl triacetate are preferred because of biocompatitibility and pharmaceutical acceptance.

Additionally, mixtures of the foregoing high and low water solubility organic liquids providing varying degrees of solubility for the matrix forming material can be used to alter the hardening rate of the implant composition. Examples include a combination of N-methyl pyrrolidone and propylene carbonate, which provides a more hydrophobic solvent than N-methyl pyrrolidone alone, and a combination of N-methyl pyrrolidone and polyethylene glycol, which provides a more hydrophilic solvent than N-methyl pyrrolidone alone.

Chemotherapeutic Agent

Suitable cell-cycle dependent biological agents, schedule-dependent biological agents, metabolites thereof, or prodrugs thereof include drugs, proteins or other molecules that block, impede, or otherwise interfere with, cell cycle progression at the G1-phase, G1/S interface, S-phase, G2/M interface, or M-phase of the cell cycle. These drugs are cell cycle-dependent or schedule-dependent.

Specifically, suitable cell-cycle dependent biological agents, schedule-dependent biological agents, metabolites thereof, or prodrugs thereof include:

(1) Analogues of uridine nucleosides, analogues of thymidine nucleosides, and analogues of uridine and thymidine nucleosides. These compounds act at the S-phase in tumor cells, and possibly neovascular endothelial cells. These compounds include, e.g., 5-fluorodeoxyuridine (floxuridine, FUDR); 5-flurouracil (5-FU); prodrugs of 5-FU (e.g. capecitabine, 5′-deoxy-5-fluorouridine, ftorafur, flucytosine); bromodeoxyuridine; iododexoyuridine; and prodrugs of halopyrimidines, including polymeric prodrugs of halopyrimidines.

(2) Modulators of fluoropyrimidines. These compounds act at the S-phase in tumor cells, and possibly neovascular endothelial cells. These compounds include, e.g., leurovorin, methotrexate and other folates; levamisole; acivicin; phosphonacetyl-L-aspartic acid (PALA); brequinar; 5-ethynyluracil; and uracil.

(3) Cytidine analogues and cytidine nucleoside analogues. These compounds act at the S-phase in tumor cells, and possibly neovascular endothelial cells. These compounds include, e.g., cytarabine (Ara-C, cytosine arabinoside); gemcitabine (2′,2′-difluorodeoxycytidine); 5-azacytidine; and prodrugs of cytidine analogues, including polymeric prodrugs of cytidine analogues.

(4) Purine analogues and purine nucleoside analogues. These compounds act at the S-phase in tumor cells, and possibly neovascular endothelial cells. These compounds include, e.g., 6-thioguanine; 6-mercaptopurine; azathioprine; adenosine arabinoside (Ara-A); 2′,2′-difluorodeoxyguanosine; deoxycoformycin (pentostatin); cladribine (2-chlorodeoxyadeno sine); inhibitors of adenosine deaminase; and prodrugs of purine analogues, including polymeric prodrugs of purine analogues.

(5) Antifolates. These compounds act at the S-phase in tumor cells, and possibly neovascular endothelial cells. These compounds include, e.g., methotrexate; aminopterin; trimetrexate; edatrexate; N10-propargyl-5,8-dideazafolic acid (CB3717); ZD1694, 5,8-dideazaisofolic acid (IAHQ); 5,10-dideazatetrahydrofolic acid (DDATHF); 5-deazafolic acid (efficient substrate for FPGS); PT523 (N alpha-(4-amino-4-deoxypteroyl)-N delta-hemiphthaloyl-L-ornithine); 10-ethyl-10-deazaaminopterin (DDATHF, lomatrexol); piritrexim; 10-EDAM; ZD1694; GW1843; PDX (10-propargyl-10-deazaaminopterin); multi-targeted folate (i.e. LY231514, permetrexed); any folate-based inhibitor of thymidylate synthase (TS); any folate-based inhibitor of dihydrofolate reductase (DHFR); any folate-based inhibitor of glycinamide ribonucleotide transformylase (GARTF); any inhibitor of folylpolyglutamate synthetase (FPGS); and any folate-based inhibitor of GAR formyl transferase (AICAR transformylase).

(6) Other antimetabolites. These compounds act at the S-phase in tumor cells, and possibly neovascular endothelial cells. These compounds include, e.g., hydroxyurea and polyamines.

(7) S-phase specific radiotoxins (deoxythymidine analogues). These compounds act at the S-phase in all cells undergoing DNA synthesis. The compounds are incorporated into chromosomal DNA during S-phase. These compounds include, e.g., [¹²⁵I]-iododeoxyuridine; [¹²³I]-iododeoxyuridine; [¹²⁴I]-iododeoxyuridine; [^{80m}Br]-iododeoxyuridine; [¹³¹I]-iododeoxyuridine; and [²¹¹At]-astatine-deoxyuridine.

(8) Inhibitors of enzymes involved in deoxynucleoside/deoxynucleotide metabolism. These compounds act at the S-phase in tumor cells, and possibly neovascular endothelial cells. These compounds include, e.g., inhibitors of thymidylate synthase (TS); inhibitors of dihydrofolate reductase (DHFR); inhibitors of glycinamide ribonucleotide transformylase (GARTF); inhibitors of folylpolyglutamate synthetase (FPGS); inhibitors of GAR formyl transferase (AICAR transformylase); inhibitors of DNA polymerases (DNA Pol; e.g. aphidocolin); inhibitors of ribonucleotide reductase (RNR); inhibitors of thymidine kinase (TK); and inhibitors of topoisomerase I enzymes (e.g. camptothecins, irinotecan [CPT-11, camptosar], topotecan, NX-211 [lurtotecan], rubitecan, etc.).

(9) DNA chain-terminating nucleoside analogues. These compounds act specifically on S-phase cells and are incorporated into chromosomal DNA during S-phase; terminate growing DNA strand. These compounds include, e.g., acyclovir; abacavir; valacyclovir; zidovudine (AZT); didanosine (ddI, dideoxycytidine); zalcitabine (ddC); stavudine (D4T); lamivudine (3TC); Any 2′3′-dideoxy nucleoside analogue; and any 2′3′-dideoxy nucleoside analogue that terminates DNA synthesis. These compounds include, e.g., inhibitors of growth factor receptor tyrosine kinases that regulate progression through the G1-phase, G1/S interface, or S-phase of the cell cycle (e.g. EGF receptors, HER-2 neu/c-erbB2 receptor, PDGF receptors, etc; [e.g. trastusumab, iressa, erbitux, tarceva]); inhibitors of non-receptor tyrosine kinases (e.g. c-src family of tyrosine kinases; [e.g. Gleevec]); inhibitors of serine-threonine kinases that regulate progression through the G1-phase, G1/S interface or S-phase of the cell cycle (e.g. G1 cyclin-dependent kinases, G1/S cyclin-dependent kinases, and S cyclin-dependent kinases [e.g. CDK2, CDK4, CDK5, CDK6]; mitogen-activated kinases; MAP kinase signaling pathway); inhibitors of G1-phase, G1/S interface or S-phase cyclins [e.g. cyclins D1, D2, D3, E, and A]); inhibitors of G-proteins and cGMP phosphodiesterases that positively regulate cell cycle progression at the G1-phase, G1/S interface or S-phase of the cell cycle; drugs that inhibit the induction of immediate early response transcription factors (e.g. N-terminal c-jun kinase, c-myc); and drugs that inhibit proteosomes that degrade ‘negative’ cell cycle regulatory molecules (e.g. p53, p27/Kip1; [e.g. bortezomib]).

(10) Cytokines, growth factors, anti-angiogenic factors and other proteins that inhibit cell cycle progression at the G1-phase or G1/S interface of the cell cycle. These compounds act at G1, G1/S or S-phase of the cell cycle in tumor cells, and in some cases, neovascular endothelial cells. These compounds include, e.g., interferons; interleukins; somatostatin and somatostatin analogues (octreotide, sandostatin LAR); and many anti-angiogenic factors inhibit cell proliferation of endothelial cells at the G1 or G1/S phases of the cell cycle.

(11) Drugs and compounds that inhibit cell cycle progression at the G2/M interface, or M-phase of the cell cycle. These compounds act at G2/M interface or M-phase of the cell cycle in tumor cells, and in some cases, neovascular endothelial cells. These compounds include, e.g., (a) microtubule-targeting drugs—taxanes (e.g., taxol, taxotere, epothilones, and other taxanes and derivatives); (b) microtubule-targeting drugs—vinca alkaloids (e.g., vinblastine, vincristine, vindesine; vinflunine, vinorelbine, vinzolidine, nocadazole, and colchicines); (c) microtubule-targeting drugs—others (e.g., estramustine, CP-248 and CP-461); (d) inhibitors of serine-threonine kinases that regulate progression through the G2/M interface or M-phase of the cell cycle (e.g., inhibitors of G2/M cyclin-dependent kinases (e.g. CDC2); inhibitors of M-phase cyclins (e.g. cyclin B) and any drug that blocks, impedes, or otherwise interferes with, cell cycle progression at the G2/M interface, or M-phase of the cell cycle).

(12) Radiopharmaceuticals useful in radiation therapy and/or diagnosis. A suitable class of radioisotopes decay by a nuclear disintegration process known as the “Auger Process” or “Auger Cascade”. Auger emitting isotopes generate short acting electrons that efficiently cleave duplex DNA. Suitable Auger-emitting radionuclides include, e.g., 125-Iodine, 123-Iodine and 80m-Bromine. Suitable corresponding halogenated pryimidine and purine nucleosides include, e.g., 5-¹²⁵Iodo-2′-deoxyuridine, 5-¹²³Iodo-2′-deoxyuridine, 5-^{80m}Bromo-2′-deoxyuridine and 8-^{80m}Bromo-2′-guanidine.

The cell-cycle biological agent, schedule-dependant biological agent, metabolite thereof, pharmaceutically acceptable salt thereof, or prodrug thereof can be incorporated into a particulate or encapsulated controlled-release component. The particulate controlled-release component can include a conjugate in which the cell-cycle biological agent, schedule-dependant biological agent, metabolite thereof, pharmaceutically acceptable salt thereof, or prodrug thereof is covalently bonded to a carrier molecule. The particulate controlled-release component can be a microstructure selected from the group of a microcapsule, a nanoparticle, a cyclodextrin, a liposome, and a micelle. Additionally, the microstructure can be of any suitable size (e.g., less than about 500 microns). Alternatively, the particulate controlled-release component can be a macrostructure selected from the group of a fiber, film, rod, disc and cylinder. Additionally, the macrostructure can be of any suitable size (e.g., at least about 500 microns).

Additional/Second Chemotherapeutic Agent

In additional to the cell-cycle dependent biological agent, schedule-dependent biological agent, metabolite thereof, or prodrug thereof described above; a second chemotherapeutic agent can be employed in the present invention. The second chemotherapeutic agent can be any suitable compound that has biological activity against one or more forms of cancer.

Suitable additional chemotherapeutic agents include, e.g., drugs that may act at various stages of the cell cycle. These drugs are not particularly cell cycle- or schedule-dependent. Such compounds include, e.g., antracyclines (e.g., doxorubicin, daunorubicin, epirubicin, idarubicin, and mitoxantrone); (b) other DNA intercalators (e.g., actinomycins C, D, B, etc.; podophyllotoxins, and epipodophyllatoxins (etoposide, teniposide, ctoposide)); (c) alkylating agents (e.g., mechlorethamine, melphalan, cyclophosphamide, chlorambucil, ifosfamide, carmustine, lomustine, busulfan, dacarbazine, cisplatin, carboplatin, oxaliplatin, iproplatin, and tetraplatin); (d) hormonal agents (e.g., antiestrogens/estrogen antagonists (tamoxifen and other SERMs); LHRH agonists and antagonists (leuprolide acetate, goserelin, abarelix); aromatase inhibitors; and antiandrogens; (e) chemoprevention agents (e.g., NSAIDs and cis-retinoids); prodrugs thereof, and metabolites thereof.

Alternatively, the additional chemotherapeutic agent can include, e.g., antineoplasts. Representative antineoplasts include, e.g., adjuncts (e.g., levamisole, gallium nitrate, granisetron, sargramostim strontium-89 chloride, filgrastim, pilocarpine, dexrazoxane, and ondansetron); androgen inhibitors (e.g., flutamide and leuprolide acetate); antibiotic derivatives (e.g., doxorubicin, bleomycin sulfate, daunorubicin, dactinomycin, and idarubicin); antiestrogens (e.g., tamoxifen citrate, analogs thereof, and nonsteroidal antiestrogens such as toremifene, droloxifene and roloxifene); antimetabolites (e.g., fludarabine phosphate, interferon alfa-2b recombinant, methotrexate sodium, plicamycin, mercaptopurine, and thioguanine); cytotoxic agents (e.g., doxorubicin, carmustine [BCNU], lomustine [CCNU], cytarabine USP, cyclophosphamide, estramucine phosphate sodium, altretamine, hydroxyurea, ifosfamide, procarbazine, mitomycin, busulfan, cyclophosphamide, mitoxantrone, carboplati, cisplati, cisplatin, interferon alfa-2a recombinant, paclitaxel, teniposide, and streptozoci); hormones (e.g., medroxyprogesterone acetate, estradiol, megestrol acetate, octreotide acetate, diethylstilbestrol diphosphate, testolactone, and goserelin acetate); immunomodulators (e.g., aldesleukin); nitrogen mustard derivatives (e.g., melphalan, chlorambucil, mechlorethamine, and thiotepa) and steroids (betamethasone sodium phosphate and betamethasone acetate).

Suitable additional chemotherapeutic agents include, e.g., alkylating agents, antimitotic agents, plant alkaloids, biologicals, topoisomerase I inhibitors, topoisomerase II inhibitors, and synthetics.

Representative alkylating agents include, e.g., asaley, AZQ, BCNU, busulfan, bisulphan, carboxyphthalatoplatinum, CBDCA, CCNU, CHIP, chlorambucil, chlorozotocin, cis -platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864. See, AntiCancer Agents by Mechanism, http://dtp.nci.nih.gov/docs/cancer/searches/standard_mechanism_list.html, Apr. 12, 1999.

Representative antimitotic agents include, e.g., allocolchicine, Halichondrin B, colchicine, colchicine derivatives, dolastatin 10, maytansine, rhizoxin, paclitaxel derivatives, paclitaxel, thiocolchicine, trityl cysteine, vinblastine sulfate, and vincristine sulfate.

Representative plant alkaloids include, e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere.

Representative biologicals include, e.g., alpha interferon, BCG, G-CSF, GM-CSF, and interleukin-2.

Representative topoisomerase I inhibitors include, e.g., camptothecin, camptothecin derivatives, and morpholinodoxorubicin.

Representative topoisomerase II inhibitors include, e.g., mitoxantron, amonafide, m-AMSA, anthrapyrazole derivatives, pyrazoloacridine, bisantrene HCL, daunorubicin, deoxydoxorubicin, menogaril, N, N-dibenzyl daunomycin, oxanthrazole, rubidazone, VM-26 and VP-16.

Representative synthetics include, e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium.

Alternatively, the additional chemotherapeutic agent can include tubulin-binding drugs and drugs that affect tubulin dynamics and function. This includes a variety of drugs that are chemically unrelated to vinca alkaloids and taxanes (e.g. CP-248 [a derivative of exisulind] and ILX-65 1). These drugs have distinctive effects on cells at G2M-phase and may have functionally independent effects on cells in G1 and/or S phase.

Alternatively, the additional chemotherapeutic agent can include selective apoptotic antineoplastic drugs (SAANDs), which include sulindac, aptosyn, CP461, CP-248 and related sulindac derived compounds that inhibit one or more of the following isozymes of cyclic GMP phosphodiesterase (cGMP PDE): 1, 2, 5.

Alternatively, the additional chemotherapeutic agent can include drugs that inhibit proteosomes (bortezomib or Velcade). Proteosomes degrade many ubiquitinated proteins that have been marked for active destruction. Ubiquitinated proteins include many critical cell cycle regulatory molecules and molecules that regulate apoptosis at specific stages of the cell cycle. While proteosomes may degrade proteins throughout the cell cycle, the proteins that are degraded by proteosomes include some of the most critical cell cycle regulatory proteins. The so-called “cell cycle active rationale” may be applied to the treatment of diseases in various categories, including cancer, inflammatory/autoimmune diseases, and neurological diseases that involve disorderly cell cycle and/or apoptosis.

Alternatively, the additional chemotherapeutic agent can include drugs that inhibit heat shock protein 90 (HSP90), a ‘chaperonin’ that participates in the degradation of ‘client’ proteins in the ubiquitin mediated proteosome pathway. Several drugs seem to exert their antitumour effect by inhibiting the intrinsic ATPase activity of HSP90, resulting in degradation of HSP90 “client proteins” via the ubiquitin proteosome pathway. Examples include: geldanamycin, 17-allylamino geldanamycin, 17-demethoxygeldanamycin and radicicol.

Growth Factors

Many growth factors and cytokines have the capacity to stimulate malignant cells to traverse specific points in the cell cycle. For example, G-CSF or GM-CSF can stimulate leukemic blasts in acute myeloid leukemia to traverse the G1/S interface. This increases the cells' susceptibility to cell-cycle specific drugs, such as cytarabine. Similar strategies have been tested using EGF and cytotoxic drugs for solid tumors. In order to respond the the growth factor, cells must be at a specific stage of the cell cycle, e.g., at the G1/S interface. The continuous presence of a growth factor could be beneficial, because at any given time, only a subset of the blasts are at G1/S. Thus, the growth factors act in a cell cycle specific fashion. Similar logic can be applied to the use of hematopoietic growth factors used to treat neutropenia, anemia and thrombocytopenia.

As such, peptide/protein growth factors can be employed in the present invention to promote survival of normal non-malignant cell lineages. One benefit in using such substances is the ability to protect proliferating cells in bone marrow, skin, oral and gastrointestinal mucosa, and hair follicles.

Examples of substances within this category include, e.g., hematopoietic growth factors: G-CSF, GM-CSF, erythropoietin, thrombopoietin and biologically active derivatives of these peptides; keratinocyte growth factor (KGF) for mucositis; B-lymphocyte stimulating pepdie (BLys); platelet derived growth factor (PDGF), epithelial growth factor (EGF), TGF-alpha and related growth factors; interleukins (e.g. IL-2, IL-6); other cytokines, growth factors and peptides that stimulate proliferation of non-malignant cells that need to be protected.

Therapeutic Growth Factors/Cytokines

Some therapeutic growth factors/cytokines can inhibit cell proliferation of cancer cells and/or neovascular cells at specific stages of the cell cycle. For example, interferons, somatostatin, octreotide and analogues thereof, thrombospondin and troponin-I inhibit neovascular endothelial cell proliferation by reducing the rate at which the cells enter S-phase. As such, any one or more of these substances can be employed in the present invention.

Prodrugs

The term “prodrug” as used herein refers to derivatives of biologically active compounds which have chemically or metabolically cleavable groups and become by solvolysis or under physiological conditions the biologically acive compounds, which are pharmaceutically active in vivo. Prodrugs are pharmacologically inactive derivatives of active drugs. They are designed to maximize the amount of active drug that reaches its site of action, through manipulation of the physicochemical, biopharmaceutical or pharmacokinetic properties of the drug. Prodrugs are converted into the active drug within the body through enzymatic or non-enzymatic reactions. Prodrugs are typically employed for one or more reasons, for example: (1) to increase site specificity of the drug, (2) to improve the drug's chemical stability, (3) to alter the drug's solubility, (4) to alter the pharmacokinetics, (5) to decrease the drug's toxicity and adverse effects, and/or (6) to alter drug transportation across tissue or membranes.

Prodrugs include hydroxyl and amino derivatives well-known to practitioners of the art, such as, for example, esters prepared by reaction of the parent hydroxyl compound with a suitable carboxylic acid, or amides prepared by reaction of the parent amino compound with a suitable carboxylic acid. Simple aliphatic or aromatic esters derived from hydroxyl groups pendent on the compounds employed in this invention are preferred prodrugs. In some cases it may be desirable to prepare double ester type prodrugs such as (acyloxy) alkyl esters or ((alkoxycarbonyl)oxy)alkyl esters. Specific suitable esters as prodrugs include methyl, ethyl, propyl, isopropyl, n-butyl, isobutyl, tert-butyl, and morpholinoethyl.

Hydrolysis in Drug and Prodrug Metabolism: Chemistry, Biochemistry, and Enzymology, by Bernard Testa and Joachim Mayer; Vch Verlagsgesellschaft Mbh (August 2003) provides a comprehensive review of metabolic reactions and enzymes involved in the hydrolysis of drugs and prodrugs. The text also describes the significance of biotransformation and discusses the physiological roles of hydrolytic enzymes, hydrolysis of amides, and the hydrolysis of lactams. Additional references useful in designing prodrugs employed in the present invention include, e.g., Biological Approaches to the Controlled Delivery of Drugs (Annals of the New York Academy of Sciences, Vol. 507), R. L. Juliano (editor) (February 1988); Design of Biopharmaceutical Properties through Prodrugs and Analogs, Edward B. Roche (editor), Amer Pharmaceutical Assn (MacK) (June 1977); Prodrugs: Topical and Ocular Drug Delivery (Drugs and the Pharmaceutical Sciences, Vol. 53), Kenneth B. Sloan (editor), Marcel Dekker (Mar. 17, 1992); Enzyme-Prodrug Strategies for Cancer Therapy, Roger G. Melton (editor), Richard J. Knox (editor), Plenum Press (February 1999); Design of Prodrugs, Hans Bundgaard (editor), Elsevier Science (February 1986); Textbook of Drug Design and Development, Povl Krogsgaard-Larsen, Hans Bundgaard (editor), Hardwood Academic Pub (May 1991); Conversion of Non-Toxic Prodrugs to Active, Anti-Neoplastic Drugs Selectively in Breast Cancer Metastases, Basse, Per H. (September 2000); and Marine lipids for produrgs, of compounds and other pharmaceutical applications, M. Másson, T. Loftsson and G. G. Haraldsson, Die Pharmazie, 55 (3), 172-177 (2000);

When the biologically active agent is a nucleoside analogue, the following references can be particularly useful in designing prodrugs of the nucleoside analogues: 5′-[2-(2-Nitrophenyl)-2-methylpropionyl]-2′-deoxy-5-fluorouridine as a potential bioreductively activated prodrug of FUDR: synthesis, stability and reductive activation, Hu L, Liu B, Hacking D R., Bioorg Med Chem Lett. 2000 Apr. 17;10(8):797-800; Specificity of esterases and structure of prodrug esters. II. Hydrolytic regeneration behavior of 5-fluoro-2′deoxyuridine (FUdR) from 3′,5′-diesters of FUdR with rat tissue homogenates and plasma in relation to their antitumor activity, Kawaguchi T, Saito M, Suzuki Y, Nambu N, Nagai T., Chem Pharm Bull (Tokyo). April 1985;33(4):1652-9; Kang et al., Nucleosides Nucleotides 17 (1998) 1089; Jiang et al., J. Biol. Chem., 273 (1998) 11017; Li et al., Tetrahedron 53 (1997) 12017; Kruppa et al., Bioorg. Med. Chem. Lett., 7 (1997) 945; U.S. Pat. No. 6,492,347; U.S. Pat. No. 5,981,507; U.S. Pat. No. 5,554,386; U.S. Pat. No. 5,424,297; U.S. Pat. No. 5,336,506; U.S. Pat. No. 5,233,031; U.S. Pat. No. 5,149,794; Benet et al., 1990, Pharmacokinetics: The Dynamics of Drug Absorption, Distribution, and Elimination, in Goodman and Gilman's The Pharmacological Basis of Therapeutics, Eigth edition, Goodman et al., eds., Pergamon Press Inc., New York, pp. 3-32; A 5-fluorodeoxyuridine prodrug as targated therapy for prostate cancer, Mhaka A, Denmeade S R, Yao W, Isaacs J T, Khan S R, Biiorg Med Chem Lett, 2002 Sep. 2; 12 (17): 2459-61; 5′-[2-(2-Nitrophenyl)-2-methylpropionyl]-2′-deoxy-5-fluorouridine as a potential bioreductively activated prodrug of FUDR: synthesis, stability and reductive activation, Hu L, Liu B. Hacking D R., Bioorg Med Chem Lett, 2000 Apr. 17;10(8):797-800; and Specificity of esterases and structure of prodrug esters. II. Hydrolytic regeneration behavior of 5-fluoro-2′-deoxyuridine (FUdR) from 3′,5′-diesters of FUdR with rat tissue homogenates and plasma in relation to their antitumor activity, Kawaguchi T, Saito M, Suzuki Y, Nambu N, Nagai T., Chem Pharm Bull (Tokyo), April 1985;33(4):1652-9.

Prodrugs employed in the present invention can include any suitable functional group that can be chemically or metabolically cleaved by solvolysis or under physiological conditions to provide the biologically acive compound (e.g., the cell-cycle dependent biological agent or schedule-dependent biological agent). Suitable functional groups include, e.g., carboxylic esters, amides, and thioesters. Depending on the reactive functional group(s) of the biologically active compound, a corresponding functional group of a suitable linker precursor can be selected from the following table, to provide, e.g., an ester linkage, thioester linkage, or amide linkage in the prodrug.

| Functional Group on | | |
|---|---|---|
| Biologically Active | Functional Group on | Resulting Linkage in |
| Compound | Linker Precursor | Prodrug |
| —COOH | —OH | Ester |
| —COOH | —NHR | Amide |
| —COOH | —SH | Thioester |
| —OH | —COOH | Carboxylic Ester |
| —SH | —COOH | Thioester |
| —NHR | —COOH | Amide |
| —OH | —OP(═O)(OH)₂ | Phosphoric Acid Ester |
| —OH | —OP(═O)(OR)₂ | Phosphoric Acid Ester |
| —OH | —SO₂OH | Sulphonic Acid Ester |

Depending on the reactive functional group(s) of the biologically active compound, one or more positions of the biologically active compound can be chosen to link the linker precursor to the biologically active compound, thereby providing the prodrug. By way of illustration, the following table shows suitable positions on several biologically active compounds (e.g., nucleoside analogues) that can be linked to a linker precursor.

| | | Suitable Positions |
|---|---|---|
| Biologically Active | Chemical Structure with | Available to Link with |
| Compound | Positions Indicated | Linker Precursor |
| 5-[125I]- iododeoxyuridine (125IUDR) | 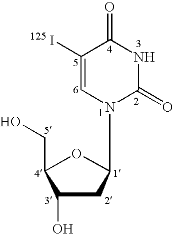 | 3′(OH) 5′(OH) 3(NH) |
| Difluorodideoxycytidine (dFdG, gemcitabine) | 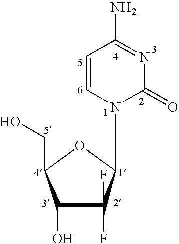 | 3′(OH) 5′(OH) 4(NH₂) |
| Deoxycoformycin (DCFM, pentostatin, nipent) | 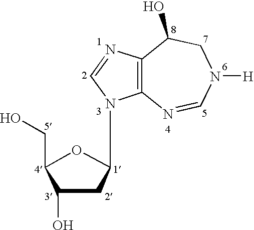 | 3′(OH) 5′(OH) 8(OH) 6(NH) |
| 6-mercaptopurine deoxynucleoside (6- MPdN) | 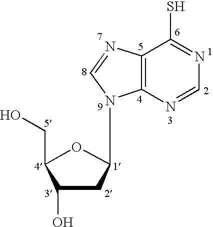 | 3′(OH) 5′(OH) 6(SH) |
| 6-thioguanine deoxynucleoside (6- TGdN) | 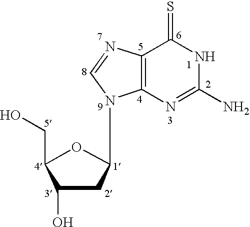 | 3′(OH) 5′(OH) 2(NH₂) |
| 5-fluorodeoxyuridine (FUDR) | 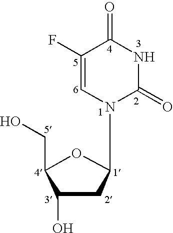 | 3′(OH) 5′(OH) 3(NH) |

Linker Precursor and Linking Group

A biologically acive compound can be linked to a suitable linker precursor to provide the prodrug. As shown above, the reactive functional groups present on the biologically active compound will typically influence the functional groups that need to be present on the linker precursor. The nature of the linker precursor is not critical, provided the prodrug employed in the present invention possesses acceptable mechanical properties and release kinetics for the selected therapeutic application. The linker precursor is typically a divalent organic radical having a molecular weight of from about 25 daltons to about 400 daltons. More preferably, the linker precursor has a molecular weight of from about 40 daltons to about 200 daltons.

The resulting linking group, present on the prodrug, may be biologically inactive, or may itself possess biological activity. The linking group can also include other functional groups (including hydroxy groups, mercapto groups, amine groups, carboxylic acids, as well as others) that can be used to modify the properties of the prodrug (e.g. for appending other molecules) to the prodrug, for changing the solubility of the prodrug, or for effecting the biodistribution of the prodrug).

Specifically, the linking group can be a divalent, branched or unbranched, saturated or unsaturated, hydrocarbon chain, having from 1 to 50 carbon atoms, wherein one or more (e.g. 1, 2, 3, or 4) of the carbon atoms is optionally replaced by (—O—) or (—NR—, wherein R can be hydrogen, alkyl, cycloalkyl alkyl, or aryl alkyl, and wherein the chain is optionally substituted on carbon with one or more (e.g. 1, 2, 3, or 4) substituents selected from the group of alkoxy, substituted alkoxy, cycloalkyl, substituted cycloalkyl, alkanoyl, alkanoyloxy, alkoxycarbonyl, alkylthio, substituted alkylthio, hydroxycarbonyl, azido, cyano, nitro, halo, hydroxy, oxo, carboxy, aryl, substituted aryl, aryloxy, substituted aryloxy, heteroaryl, substituted heteroaryl, heteroaryloxy, substituted heteroaryloxy, COOR, or NRR, wherein each R can independently be hydrogen, alkyl, cycloalkyl alkyl, or aryl alkyl.

The term “alkyl” refers to a monoradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, sec-butyl, n-hexyl, n-decyl, tetradecyl, and the like.

The alkyl can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “alkylene” refers to a diradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methylene, ethylene, n-propylene, iso-propylene, n-butylene, iso-butylene, sec-butylene, n-hexylene, n-decylene, tetradecylene, and the like.

The alkylene can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “alkoxy” refers to the groups alkyl—O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkoxy, halo, haloalkyl, hydroxy, hydroxyalkyl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkoxy, halo, haloalkyl, hydroxy, hydroxyalkyl, aryl, heteroaryl, heterocycle, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 14 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkoxy, halo, haloalkyl, hydroxy, hydroxyalkyl, aryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano

The term “heterocycle” refers to a saturated or partially unsaturated ring system, containing at least one heteroatom selected from the group oxygen, nitrogen, and sulfur, and optionally substituted with alkyl or C(═O)ORb, wherein Rb is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “nitro” refers to —NO₂.

The term “trifluoromethyl” refers to —CF₃.

The term “trifluoromethoxy” refers to —OCF₃.

The term “cyano” refers to —CN.

The term “hydroxy” refers to —OH.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano. When a substituent is keto (i.e., ═O) or thioxo (i.e., ═S) group, then 2 hydrogens on the atom are replaced.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Specifically, the linking group can be a divalent peptide, amino acid, fatty acid, saccharide, polysaccharide, polyalcohol (e.g., PEG or PVA), starch, dextrin, maltodextrin, cyclodextrin, or carbohydrate. For example, the linking group can be a divalent peptide, amino acid, saccharide, polysaccharide, or polyalcohol.

In one specific embodiment of the present invention, the linking group itself can have biological activity. For example, the linking group can be a divalent bioactive peptide such as growth hormone releasing peptide (GHRP), luteinizing hormone-releasing hormone (LHRH), leuprolide acetate, somatostatin, bombesin, gastrin releasing peptide (GRP), calcitonin, bradykinin, galanin, melanocyte stimulating hormone (MSH), growth hormone releasing factor (GRF), amylin, tachykinins, secretin, parathyroid hormone (PTH), enkephalin, endothelin, calcitonin gene releasing peptide (CGRP), neuromedins, parathyroid hormone related protein (PTHrP), glucagon, neurotensin, adrenocorticotrophic hormone (ACTH), peptide YY (PYY), glucagon releasing peptide (GLP), vasoactive intestinal peptide (VIP), pituitary adenylate cyclase activating peptide (PACAP), motilin, substance P, neuropeptide Y (NPY), TSH, and analogs and fragments thereof. See, e.g., U.S. Pat. Nos. 6,221,958; 6,113,943; and 5,863,985.

In one specific embodiment of the present invention, the linking group can be lipophillic. In another specific embodiment of the present invention, the linking group can be hydrophilic.

A suitable class of prodrugs include compounds of formula (I):
D-X¹-L¹ (I)

wherein,

D is a mono radical of a biologically acive compound disclosed herein (e.g., a cell-cycle dependent biological agent or a schedule-dependent biological agent);

X¹ is a carboxylic ester linkage, an amide linkage, a thioester linkage, a phosphoric acid ester linkage, or a sulphonic acid ester linkage; and

L¹ is a linking group.

Another suitable class of prodrugs include compounds of formula (II):

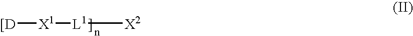

wherein,

each D is independently a mono- or di-radical of a biologically acive compound disclosed herein (e.g., a cell-cycle dependent biological agent or a schedule-dependent biological agent);

each X¹ is independently a carboxylic ester linkage, an amide linkage, a thioester linkage, a phosphoric acid ester linkage, or a sulphonic acid ester linkage;

each L¹ is independently a linking group;

X² is a carboxylic ester, an amide, a thioester, a phosphoric acid ester, or a sulphonic acid ester; and

n is about 1 to about 10,000.

As shown above, a suitable class of prodrugs includes polymeric prodrugs of biologically active compounds disclosed herein (e.g., a cell-cycle dependent biological agent or a schedule-dependent biological agent). Depending on the reactive functional group(s) of the biologically active compound, one or more positions of the biologically active compound can be chosen to link the linker precursor to the biologically active compound, in a repeated fashion, thereby providing the polymeric prodrug. By way of illustration, the following table shows suitable exemplary positions and linkages on several biologically active compounds (e.g., nucleoside analogues) that can be linked to a linker precursor, to provide the polymeric prodrug.

| | | Suitable Linkages |
|---|---|---|
| Biologically Active | Chemical Structure with | Available to Link with |
| Compound | Positions Indicated | Linker Precursor |
| 5-[125I]- iododeoxyuridine (125IUDR) | 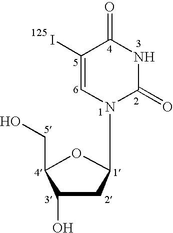 | 3′(OH)→3′(OH) 3′(OH)→5′(OH) 3′(OH)→3′(NH) 5′(OH)→5′(OH) 5′(OH)→3′(OH) 5′(OH)→3′(NH) 3(NH)→3(NH) 3(NH)→3′(OH) 3(NH)→5′(OH) |
| Difluorodideoxycytidine (dFdG, gemcitabine) | 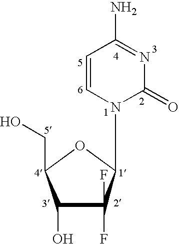 | 3′(OH)→3′(OH) 3′(OH)→5′(OH) 3′(OH)→4(NH₂) 5′(OH)→5′(OH) 5′(OH)→3′(OH) 5′(OH)→4(NH₂) 4(NH₂)→4(NH₂) 4(NH₂)→3′(OH) 4(NH₂)→5′(OH) |
| Deoxycoformycin (DCFM, pentostatin, nipent) | 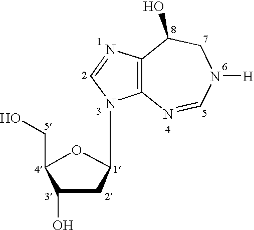 | 3′(OH)→3′(OH) 3′(OH)→5′(OH) 3′(OH)→8(OH) 3′(OH)→6(NH) 5′(OH)→3′(OH) 5′(OH)→8(OH) 5′(OH)→6(NH) 5′(OH)→5′(OH) 8(OH)→8(OH) 8(OH)→3′(OH) 8(OH)→5′(OH) 8(OH)→6(NH) 6(NH)→6(NH) 6(NH)→8(OH) 6(NH)→3′(OH) 6(NH)→5′(OH) |
| 6-mercaptopurine deoxynucleoside (6- MPdN) | 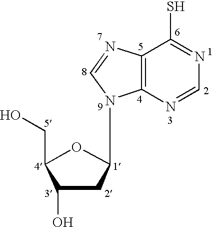 | 3′(OH)→3′(OH) 3′(OH)→5′(OH) 3′(OH)→6(SH) 5′(OH)→5′(OH) 5′(OH)→3′(OH) 5′(OH)→6(SH) 6(SH)→6(SH) 6(SH)→3′(OH) 6(SH)→5′(OH) |
| 6-thioguanine deoxynucleoside (6- TGdN) | 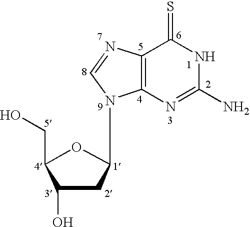 | 3′(OH)→3′(OH) 3′(OH)→5′(OH) 3′(OH)→2(NH₂) 5′(OH)→5′(OH) 5′(OH)→3′(OH) 5′(OH)→2(NH₂) 2(NH₂)→2(NH₂) 2(NH₂)→3′(OH) 2(NH₂)→5′(OH) |
| 5-fluorodeoxyuridine (FUDR) | 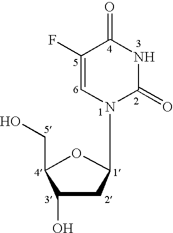 | 3′(OH)→3′(OH) 3′(OH)→5′(OH) 3′(OH)→3(NH) 5′(OH)→5′(OH) 5′(OH)→3′(OH) 5′(OH)→3(NH) 3(NH)→3(NH) 3(NH)→3′(OH) 3(NH)→5′(OH) |

Dosages

The flowable composition is a liquid or a gel composition, suitable for injection into a patient. As such, the flowable composition can preferably be formulated as an injectable subcutaneous delivery system. The amount of flowable composition administered will typically depend upon the desired properties of the controlled release implant. For example, the amount of flowable composition can influence the length of time in which the cell-cycle dependent biological agent, a schedule-dependent biological agent, a metabolite thereof, or a prodrug thereof is released from the controlled release implant. Additionally, the amount of flowable composition administered will typically depend upon the specific intended use (e.g., nature and stage/progression of the cancer). Additionally, the amount of flowable composition administered will typically depend upon the number of controlled release implants formed (i.e., the number of flowable compositions administered). Specifically, up to about 200, up to about 100, up to about 50, up to about 25, or up to about 10 flowable compositions can be administered and up to about 200, up to about 100, up to about 50, up to about 25, or up to about 10 controlled release implants can be formed by the administration of those flowable compositions. Typically, as the number of flowable compositions administered increases, the amount of flowable composition administered will decrease. Likewise, as the number of flowable compositions administered decreases, the amount of flowable composition administered will typically increase.

Specifically, the composition can be used to formulate a one year delivery system of cell-cycle dependent biological agent, schedule-dependent biological agent, metabolite thereof, pharmaceutically acceptable salt thereof, or prodrug thereof. The composition can also be used to formulate a six month delivery system of cell-cycle dependent biological agent, schedule-dependent biological agent, metabolite thereof, pharmaceutically acceptable salt thereof, or prodrug thereof. The composition can also be used to formulate a three month delivery system of cell-cycle dependent biological agent, schedule-dependent biological agent, metabolite thereof, pharmaceutically acceptable salt thereof, or prodrug thereof. The composition can also be used to formulate a two month delivery system of cell-cycle dependent biological agent, schedule-dependent biological agent, metabolite thereof, pharmaceutically acceptable salt thereof, or prodrug thereof. The composition can also be used to formulate a one month delivery system of cell-cycle dependent biological agent, schedule-dependent biological agent, metabolite thereof, pharmaceutically acceptable salt thereof, or prodrug thereof.

Specifically, up to about 10 mL of the flowable composition can be administered. More specifically, up to about 5 mL, up to about 1 mL, or up to about 0.5 mL of the flowable composition can be administered.

When multiple controlled release implants are formed (i.e., multiple flowable compositions are administered) as described above, each flowable composition administered can include the same amount of cell-cycle dependent biological agent, schedule-dependent biological agent, metabolite thereof, pharmaceutically acceptable salt thereof, or prodrug thereof. Alternatively, when multiple controlled release implants are formed (i.e., multiple flowable compositions are administered) as described above, each flowable composition administered can include a different amount of cell-cycle dependent biological agent, schedule-dependent biological agent, metabolite thereof, pharmaceutically acceptable salt thereof, or prodrug thereof. Each of the flowable compositions can be administered in any suitable amount. Specifically, each of the flowable composition administered can be up to about 10 mL, up to about 5 mL, up to about 1 mL, up to about 0.5 mL, or up to about 0.1 mL.

The cell-cycle dependent biological agent, schedule-dependent biological agent, metabolite thereof, pharmaceutically acceptable salt thereof, or prodrug thereof can be present in any effective, suitable and appropriate amount. For example, the cell-cycle dependent biological agent, schedule-dependent biological agent, metabolite thereof, pharmaceutically acceptable salt thereof, or prodrug thereof can be present up to about 70 wt. % of the flowable composition, up to about 60 wt. % of the flowable composition, up to about 40 wt. % of the flowable composition, or up to about 20 wt. % of the flowable composition. Specifically, the cell-cycle dependent biological agent, schedule-dependent biological agent, metabolite thereof, pharmaceutically acceptable salt thereof, or prodrug thereof can be present up to about 10 wt. % of the flowable composition, up to about 5 wt. % of the flowable composition, up to about 1 wt. % of the flowable composition, or up to about 0.1 wt. % of the flowable composition.

As described above, when multiple controlled release implants are formed (i.e., multiple flowable compositions are administered), each flowable composition administered can include the same amount of cell-cycle dependent biological agent, schedule-dependent biological agent, metabolite thereof, pharmaceutically acceptable salt thereof, or prodrug thereof. Alternatively, when multiple controlled release implants are formed (i.e., multiple flowable compositions are administered), each flowable composition administered can include a different amount of cell-cycle dependent biological agent, schedule-dependent biological agent, metabolite thereof, pharmaceutically acceptable salt thereof, or prodrug thereof. In any event, each of the flowable composition administered can independently include the cell-cycle dependent biological agent, schedule-dependent biological agent, metabolite thereof, pharmaceutically acceptable salt thereof, or prodrug thereof in up to about 10 wt. % of the flowable composition, up to about 5 wt. % of the flowable composition, up to about 1 wt. % of the flowable composition, or up to about 0.1 wt. % of the flowable composition.

Specicfically, the flowable composition can have a volume of more than about 0.001 mL. Additionally, the flowable composition can have a volume of up to about 20.0 mL. Specifically, the flowable composition can have a volume of about 0.01 mL to about 10.0 mL, about 0.05 mL to about 1.5 mL, about 0.1 mL to about 1.0 mL, or about 0.2 mL to about 0.8 mL.

Specifically, the flowable composition can be formulated for administration less than about once per day. More specifically, the flowable composition can be formulated for administration less than about once per week, less than about once per month, more than about once per year, about once per week to about once per year, or about once per month to about once per year.

The flowable composition will effectively deliver the cell-cycle biological agent, schedule-dependant biological agent, metabolite thereof, pharmaceutically acceptable salt thereof, or prodrug thereof to mammalian tissue at a suitable, effective, safe, and appropriate dosage. For example, the flowable composition can effectively deliver the cell-cycle biological agent, schedule-dependant biological agent, metabolite thereof, pharmaceutically acceptable salt thereof, or prodrug thereof to mammalian tissue at a dosage of more than about 0.001 picogram/kilogram/day, more than about 0.01 picogram/kilogram/day, more than about 0.1 picogram/kilogram/day, or more than about 1 picogram/kilogram/day. Alternatively, the flowable composition can effectively deliver the cell-cycle biological agent, schedule-dependant biological agent, metabolite thereof, pharmaceutically acceptable salt thereof, or prodrug thereof to mammalian tissue at a dosage of up to about 100 milligram/kilogram/day, up to about 50 milligram/kilogram/day, up to about 10 milligram/kilogram/day, or up to about 1 milligram/kilogram/day.

More specifically, the flowable composition can effectively deliver the cell-cycle biological agent, schedule-dependant biological agent, metabolite thereof, pharmaceutically acceptable salt thereof, or prodrug thereof to mammalian tissue at a dosage of about 0.001 picogram/kilogram/day to about 100 milligram/kilogram/day; about 0.01 picogram/kilogram/day to about 50 milligram/kilogram/day; about 0.1 picogram/kilogram/day to about 10 milligram/kilogram/day; or about 1 picogram/kilogram/day to about 1 milligram/kilogram/day.

The cell-cycle biological agent, schedule-dependant biological agent, metabolite thereof, pharmaceutically acceptable salt thereof, or prodrug thereof can be released from the controlled-release implant in any suitable manner. For example, the cell-cycle biological agent, schedule-dependant biological agent, metabolite thereof, pharmaceutically acceptable salt thereof, or prodrug thereof can be released from the controlled-release implant with linear or first order kinetics. Alternatively, the cell-cycle biological agent, schedule-dependant biological agent, metabolite thereof, pharmaceutically acceptable salt thereof, or prodrug thereof can be released from the controlled-release implant in a continuous zero order. Additionally, the cell-cycle biological agent, schedule-dependant biological agent, metabolite thereof, pharmaceutically acceptable salt thereof, or prodrug thereof can be released from the controlled-release implant with little or no drug burst.

The delivery of the cell-cycle biological agent, schedule-dependant biological agent, metabolite thereof, pharmaceutically acceptable salt thereof, or prodrug thereof to the mammalian tissue can be systemic and/or local. Specifically, the dosage can be deleivered locally. More specifically, the dosage can be delivered locally for a period of time of up to about 1 year. More specifically, the dosage can be delivered locally for a period of time of up to about 1 month, up to about 1 week, or more than about 1 day.

In addition to the cell-cycle biological agent, schedule-dependant biological agent, metabolite thereof, pharmaceutically acceptable salt thereof, or prodrug thereof; the flowable composition and/or the implant of the present invention can optionally include at least one of an analgesic, anesthetic, anti-infective agent, gastrointestinal agent, anti-migraine agent, muscle relaxant, or sedative and hypnotic. The analgesic, anesthetic, anti-infective agent, gastrointestinal agent, anti-migraine agent, muscle relaxant, or sedative and hypnotic can be present in any suitable amount. See, e.g., Physician's Desk Reference, 55^{th} Edition (2001).

Suitable analgesics include, e.g., acetaminophen, phenylpropanolamine HCl, chlorpheniramine maleate, hydrocodone bitartrate, acetaminophen elixir, diphenhydramine HCl, pseudoephedrine HCl, dextromethorphan HBr, guaifenesin, doxylamine succinate, pamabron, clonidine hydrochloride, tramadol hydrochloride, carbamazepine, sodium hyaluronate, lidocaine, hylan, Arnica Montana, radix (mountain arnica), Calendula officinalis (marigold), Hamamelis (witch hazel), Millefolium (milfoil), Belladonna (deadly nightshade), Aconitum napellus (monkshood), Chamomilla (chamomile), Symphytum officinale (comfrey), Bellis perennis (daisy), Echinacea angustifolia (narrow-leafed cone flower), Hypericum perforatum (St. John's wort), Hepar sulphuris calcareum (calcium sulfide), buprenorphine hydrochloride, nalbuphine hydrochloride, pentazocine hydrochloride, acetylsalicylic acid, salicylic acid, naloxone hydrochloride, oral transmucosal fentanyl citrate, morphine sulfate, propoxyphene napsylate, propoxyphene hydrochloride, meperidine hydrochloride, hydromorphone hydrochloride, fentanyl transdermal system, levorphanol tartrate, promethazine HCl , oxymorphone hydrochloride, levomethadyl acetate hydrochloride, oxycodone HCl, oxycodone, codeine phosphate, isometheptene mucate, dichloralphenazone, butalbital, naproxen sodium, diclofenac sodium, misoprostol, diclofenac potassium, celecoxib, sulindac, oxaprozin, salsalate, diflunisal, naproxen, piroxicam, indomethacin, indomethacin sodium trihydrate, etodolac, meloxicam, ibuprofen, fenoprofen calcium, ketoprofen, mefenamic acid, nabumetone, tolmetin sodium, ketorolac tromethamine, choline magnesium trisalicylate, and rofecoxib.

Suitable anesthetics include: propofol, halothane, desflurane, midazolam HCl, epinephrine, levobupivacaine, etidocaine hydrochloride, ropivacaine HCl, chloroprocaine HCl, bupivacaine HCl, and lidocaine HCl.

Suitable anti-infective agents include, e.g., trimethoprim, sulfamethoxazole, clarithromycin, ganciclovir sodium, ganciclovir, daunorubicin citrate liposome, fluconazole, doxorubicin HCl liposome, foscarnet sodium, interferon alfa-2b, atovaquone, rifabutun, trimetrexate glucoronate, itraconazole, ciclofovir, azithromycin, delavirdine mesylate, efavirenz, nevirapine, lamivudine/zidovudine, zalcitabine, didanosine, stavudine, abacavir sulfate, amprenavir, indinavir sulfate, saquinavir, saquinavir mesylate, ritonavir, nelfinavir, chloroquine hydrochloride, metronidazole, metronidazole hydrochloride, iodoquinol, albendazole, praziquantel, thiabendazole, ivermectin, mebendazole sulfate, tobramycin sulfate, tobramycin, azetreonam, cefotetan disodium, cefotetan, loracarbef, cefoxitin, meropenem, imipenemand cilastatin, cefazolin, cefaclor, ceftibuten, ceftizoxime, cefoperazone, cefuroxumeaxetil, cefprozil, ceftazidime, cefotaxime sodium, cefadroxil monohydrate, cephalexin, cephalexin hydrochloride, cefuroxime, cefazolin, cefamandole nafate, cefapime hydrochloride, cefdinir, ceftriaxone sodium, cefixme, cefpodoxime proxetil, dirithromycin, erythromycin, erythromycin ethylsuccinate, erythromycin stearate, erythromycin, sulfisoxazole acetyl, troleandomycin, azithromycin, clindamycin, clindamycin hydrochloride, colistimethate sodium, quinupristin/dalfopristin, vancomycin hydrochloride, amoxicillin, amoxicillin/calvulanate/potassium, penicillin G benzathine, penicillin G procaine, penicillin G potassium, carbenicillin indanyl sodium, piperacillin sodium, ticarcillin disodium, clavulanate potassium, ampicillin sodium/sulbactam sodium, tazobactam sodium, tetracycline HCl, demeclocycline hydrochloride, doxycycline hyclate, minocycline HCl, doxycycline monohydrate, oxytetracycline HCl, hydrocortisone acetate, doxycycline calcium, amphotericin B lipid, flucytosine, griseofulvin, terbinafine hydrochloride, ketoconazole, chloroquine hydrochloride, chloroquine phosphate, pyrimethamine, mefloquine hydrochloride, atovaquone and proguanil hydrochloride, hydroxychloroquine sulfate, ethambutol hydrochloride, aminosalicylic acid, rifapentine, rifampin, isoniazid, pyrazinamide, ethionamide, interferon alfa-n3, famciclovir, rimantadine hydrochloride, foscamet sodium, interferon alfacon-1, ribavirin, zanamivir, amantadine hydrochloride, palivizumab, oseltamivir phosphate, valacyclovir hydrochloride, nelfinavir mesylate, stavudine, acyclovir, acyclovir sodium, rifabutin, trimetrexate glucuronate, linezolid, moxifloxacin, moxifloxacin hydrochloride, ciprofloxacin, ciprofloxacin hydrochloride, ofloxacin, levofloxacin, lomefloxacin hydrochloride, nalidixic acid, norfloxacin, enoxacin, gatifloxacin, trovafloxacin mesylate, alatrofloxacin, sparfloxacin, aztreonam, nitrofurantoin monohydrate/macrocrystals, cefepime hydrochloride, fosfomycin tromethamine, neomycin sulfate-polymyxin B sulfate, imipenem, cilastatin, methenamine, methenamine mandelate, phenyl salicylate, atropine sulfate, hyoscyamine sulfate, benzoic acid, oxytetracycline hydrochloride, sulfamethizole, phenazopyridine hydrochloride, and sodium acid phosphate, monohydrate.

Suitable gastrointestinal agents include, e.g., alumina, magnesia, and simethicone, aluminum hydroxide, magnesium hydroxide, calcium carbonate, magnesium oxide, elemental magnesium, glycopyrrolate, trizyme, lipase, hyoscyamine sulfate, atropine sulfate, phenobarbital, loperamide hydrochloride, diphenoxylate hydrochloride, alosetron hydrochloride, defenoxin hydrochloride, bismuth subsalicylate, octreotide acetate, meclizine HCl, dolasetron mesylate, hydroxyzine hydrochloride, diphenhydramine hydrochloride, meclizine hydrochloride, prochlorperazine, granisetron hydrochloride, dronabinol, promethazine HCl, metochlopramide, chlorpromazine, trimethobenzamine hydrochloride, scopolamine, perphenazine, hydroxyzine pamoate, ondansetron hydrochloride, loperamide HCl, mesalamine, sulfasalazine, balsalazide disodium, hydrocortisone, olsalazine sodium, hyoscyamine, scopolamine hydrobromide, bisacodyl, monobasic sodium phosphate monohydrate, dibasic sodium phosphate heptahydrate, mineral oil, PEG-3350, electrolytes, extract of senna concentrate, diclofenac sodium, misoprostol, pancrelipase, pancreatin, lactase enzymes, sucaralfate, nizatidine, famotidine, cimetidine hydrochloride, ranitidine hydrochloride, psyllium husk, docusate sodium, polyethylene glycol, casanthrol, glycerin, lactulose, celecoxib, lansoprazole, amoxicillin, clarithromycin, infliximab, ursodiol, misoprostol, rabeprazole sodium, lansoprazole, and pantoprazole sodium.

Suitable homeopathic remedies include, e.g., cocculus indicus, conium maculatum, ambra grisea, and petroleum.

Suitable anti-migraine agents include, e.g., timolol maleate, propranolol hydrochloride, dihydroergotamine mesylate, ergotamine tartrate, caffeine, divalproex sodium, acetaminophen, acetylsalicylic acid, salicylic acid, naratriptan hydrochloride, sumatriptan succinate, sumatriptan, rizatriptan benzoate, and zolmitriptan.

Suitable muscle relaxants include, e.g., succinylcholine chloride, vecuronium bromide, rapacuronium bromide, rocuronium bromide, dantrolene sodium, cyclobanzaprine HCl, orphenadrine citrate, chlorzoxazone, methocarbamol, acetylsalicylic acid, salicylic acid, metaxalone, carisoprodol, codeine phosphate, diazepam, and tizanidine hydrochloride.

Suitable sedatives and hypnotics include, e.g., mephobarbital, pentobarbital sodium, lorazepam, triazolam, estazolam, diazepam, midazolam HCl, zolpidem tartrate, melatonin, vitamin B12, folic acid, propofol, meperidine HCl, promethazine HCl, diphenhydramine HCl, zaleplon, and doxylamine succinate.

The flowable composition and/or the implant of the present invention can further include at least one of: a release rate modification agent for controlling the rate of release of the cell-cycle biological agent or schedule-dependant biological agent in vivo from an implant matrix; a pore-forming agent; a biodegradable, crystallization-controlling agent; a plasticizer; a leaching agent; a penetration enhancer; an absorption altering agent; an opacification agent; and a colorant.

Release Rate Modification Agent

Rate modifying agents, plasticizers and leachable agents can be included to manage the rate of release of bioactive agent and the pliability of the matrix. Known plasticizers as well as organic compounds that are suitable for secondary pseudobonding in polymer systems are acceptable as pliability modifiers and leaching agents. Generally these agents are esters of mono, di and tricarboxylic acids, diols and polyols, polyethers, non-ionic surfactants, fatty acids, fatty acid esters, oils such as vegetable oils, and the like. The concentrations of such agents within the solid matrix can range in amount up to 60 wt % relative to the total weight of the matrix, preferably up to 30 wt % and more preferably up to 15 wt %. Generally, these leaching agents, plasticizers and pliability modifiers and their application are described in U.S. Pat. Nos. 5,702,716 and 5,447,725, the disclosures of which are incorporated herein by reference with the proviso that the polymers to be used are the biocompatible, biodegradable, thermoplastic polymers of the present invention.

A release rate modification agent may also be included in the flowable composition for controlling the rate of breakdown of the implant matrix and/or the rate of release of a bioactive agent in vivo from the implant matrix. The rate modifying agent can increase or retard the rate of release depending upon the nature of the rate modifying agent incorporated into the solid matrix according to the invention. Examples of suitable substances for inclusion as a release rate modification agent include dimethyl citrate, triethyl citrate, ethyl-heptanoate, glycerin, hexanediol, and the like.

The polymer solution may include a release rate modification agent to provide controlled, sustained release of a bioactive agent from the implant matrix. Although not intended to be a limitation to the present disclosure, it is believed the release rate modification agent alters the release rate of a bioactive agent from the implant matrix by changing the hydrophobicity of the polymer implant.

The use of a release rate modification agent may either decrease or increase the release of the bioactive agent in the range of multiple orders of magnitude (e.g., 1 to 10 to 100), preferably up to a ten-fold change, as compared to the release of a bioactive agent from a solid matrix without the release rate modification agent. Release rate modification agents which are hydrophilic, such as polyethylene glycol, may increase the release of the bioactive agent. By an appropriate choice of the polymer molecular weight in combination with an effective amount of the release rate modification agent, the release rate and extent of release of a bioactive agent from the implant matrix may be varied, for example, from relatively fast to relatively slow.

Useful release rate modification agents include, for example, organic substances which are water-soluble, water-miscible, or water insoluble (i.e., water immiscible), with water-insoluble substances preferred.

The release rate modification agent is preferably an organic compound which will substitute as the complementary molecule for secondary valence bonding between polymer molecules, and increases the flexibility and ability of the polymer molecules to slide past each other. Such an organic compound preferably includes a hydrophobic and a hydrophilic region so as to effect secondary valence bonding. It is preferred that a release rate modification agent is compatible with the combination of polymers and solvent used to formulate polymer solution. It is further preferred that the release rate modification agent is a pharmaceutically-acceptable substance.

Useful release rate modification agents include, for example, fatty acids, triglycerides, other like hydrophobic compounds, organic solvents, plasticizing compounds and hydrophilic compounds. Suitable release rate modification agents include, for example, esters of mono-, di-, and tricarboxylic acids, such as 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, and the like; polyhydroxy alcohols, such as propylene glycol, polyethylene glycol, glycerin, sorbitol, and the like; fatty acids; triesters of glycerol, such as triglycerides, epoxidized soybean oil, and other epoxidized vegetable oils; vegetable oils obtained from seeds, flowers, fruits, leaves, or stem of a plant or tree, such as sesame oil, soybean oil, cotton seed oil, almond oil, sunflower oil, and peanut oil; sterols, such as cholesterol; alcohols, such as C₆-C₁₂ alkanols, 2-ethoxyethanol, and the like. The release rate modification agent may be used singly or in combination with other such agents. Suitable combinations of release rate modification agents include, for example, glycerin/propylene glycol, sorbitol/glycerine, ethylene oxide/propylene oxide, butylene glycol/adipic acid, and the like. Preferred release rate modification agents include dimethyl citrate, triethyl citrate, ethyl heptanoate, glycerin, and hexanediol.

The amount of the release rate modification agent included in the polymer solution will vary according to the desired rate of release of the bioactive agent from the implant matrix. Preferably, the polymer solution contains about 0.5-15%, preferably about 5-10%, of a release rate modification agent.

Pore Forming Agent/Additive

The flowable composition of the present invention can be used for implantation, injection, or otherwise placed totally or partially within the body. One of the biologically active substances of the composition (e.g., cell-cycle biological agent, schedule-dependant biological agent, metabolite thereof, or prodrug thereof) and the polymer of the invention may form a homogeneous matrix, or one of the biologically active substances may be encapsulated in some way within the polymer. For example, the one of the biologically active substances may be first encapsulated in a microsphere and then combined with the polymer in such a way that at least a portion of the microsphere structure is maintained. Alternatively, one of the biologically active substances may be sufficiently immiscible in the polymer of the invention that it is dispersed as small droplets, rather than being dissolved, in the polymer. Either form is acceptable, but it is preferred that, regardless of the homogeneity of the composition, the release rate of that biologically active substance in vivo remain controlled, at least partially as a function of hydrolysis of the ester bond of the polymer upon biodegradation.

Additives can be used to advantage in further controlling the pore size in the solid matrix, which influences the structure of the matrix and the release rate of a bioactive agent or the diffusion rate of body fluids. For example, if the flowable composition is too impervious to aqueous medium, water or tissue ingrowth, a pore-forming agent can be added to generate additional pores in the matrix. Any biocompatible water-soluble material can be used as the pore-forming additive. These additives can be either soluble in the flowable composition or simply dispersed within it. They are capable of dissolving, diffusing or dispersing out of both the coagulating polymer matrix whereupon pores and microporous channels are generated. The amount of pore-forming additive (and size of dispersed particles of such pore-forming agent, if appropriate) within the flowable composition will directly affect the size and number of the pores in the polymer matrix.

Pore-forming additives include any pharmaceutically acceptable organic or inorganic substance that is substantially miscible in water and body fluids and will dissipate from the forming and formed matrix into aqueous medium or body fluids or water-immiscible substances that rapidly degrade to water soluble substances. It is further preferred that the pore-forming additive is miscible or dispersible in the organic solvent to form a uniform mixture. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores can be varied over a wide range by changing the molecular weight and percentage of pore-forming additive incorporated into the flowable composition.

As indicated, upon contact with body fluid, the solvent and optional pore-forming additive dissipate into surrounding tissue fluids. This causes the formation of microporous channels within the coagulating polymer matrix. Optionally, the pore-forming additive may dissipate from the matrix into the surrounding tissue fluids at a rate slower than that of the solvent, or be released from the matrix over time by biodegradation or bioerosion of the matrix. Preferably, the pore-forming additive dissipates from the coagulating implant matrix within a short time following implantation such that a matrix is formed with a porosity and pore structure effective to perform the particular purpose of the implant, as for example, a barrier system for a tissue regeneration site, a matrix for timed-release of a drug or medicament, and the like.

Porosity of the solid polymer matrix may be varied by the concentration of water-soluble or water-miscible ingredients, such as the solvent and/or pore-forming agent, in the polymer composition. For example, a high concentration of water-soluble substances in the flowable composition may produce a polymer matrix having a high degree of porosity. The concentration of the pore-forming agent relative to polymer in the composition may be varied to achieve different degrees of pore-formation, or porosity, in the matrix. Generally, the polymer composition will include about 0.01-1 gram of pore-forming agent per gram polymer.

The size or diameter of the pores formed in the matrix of the implant may be modified according to the size and/or distribution of the pore-forming agent within the polymer matrix. For example, pore-forming agents that are relatively insoluble in the polymer mixture may be selectively included in the polymer composition according to particle size in order to generate pores having a diameter that corresponds to the size of the pore-forming agent. Pore-forming agents that are soluble in the polymer mixture may be used to vary the pore size and porosity of the implant matrix by the pattern of distribution and/or aggregation of the pore-forming agent within the polymer mixture and coagulating and solid polymer matrix.

Pore diameter and distribution within the polymer matrix of the implant may be measured, as for example, according to scanning electron microscopy methods by examination of cross-sections of the polymer matrix. Porosity of the polymer matrix may be measured according to suitable methods known in the art, as for example, mercury intrusion porosimetry, specific gravity or density comparisons, calculation from scanning electron microscopy photographs, and the like. Additionally, porosity may be calculated according to the proportion or percent of water-soluble material included in the polymer composition. For example, a polymer composition which contains about 30% polymer and about 70% solvent and/or other water-soluble components will generate an implant having a polymer matrix of about 70% porosity.

The biologically active substance of the composition and the polymer of the invention may form a homogeneous matrix, or the biologically active substance may be encapsulated in some way within the polymer. For example, the biologically active substance may be first encapsulated in a microsphere and then combined with the polymer in such a way that at least a portion of the microsphere structure is maintained. Alternatively, the biologically active substance may be sufficiently immiscible in the polymer of the invention that it is dispersed as small droplets, rather than being dissolved, in the polymer. Either form is acceptable, but it is preferred that, regardless of the homogeneity of the composition, the release rate of the biologically active substance in vivo remain controlled, at least partially as a function of hydrolysis of the ester bond of the polymer upon biodegradation.

The article of the invention is designed for implantation or injection into the body of a mammal. It is particularly important that such an article result in minimal tissue irritation when implanted or injected into vasculated tissue. As a structural medical device, the polymer compositions of the invention provide a physical form having specific chemical, physical, and mechanical properties sufficient for the application and a composition that degrades in vivo into non-toxic residues.

The implant formed within the injectable polymer solution will slowly biodegrade within the body and allow natural tissue to grow and replace the impact as it disappears. The implant formed from the injectable system will release the drug contained within its matrix at a controlled rate until the drug is depleted. With certain drugs, the polymer will degrade after the drug has been completely released. With other drugs such as peptides or proteins, the drug will be completely released only after the polymer has degraded to a point where the non-diffusing drug has been exposed to the body fluids.

Biodegradable, Crystallization-Controlling Agent

A crystallization-controlling agent may optionally be combined with the polymer to effect homogeneity of the polymer mass, that is, a substantially uniform distribution of crystalline sections of the polymer to achieve a homogeneous mass having the desired physical characteristics of moldability, cohesion, and stability for effective use with bone and other tissues. The crystallization-controlling agent may be in the form of a dispersed solid particle in the composition, for example, an inorganic salt such as calcium carbonate or calcium phosphate, a polymer such as poly(vinyl alcohol), starch or dextran, and other like substance. Other useful crystallization-controlling agent are those substances that are either melted with the polymer during the compounding process, or soluble in the molten polymer. Examples of those substances include low molecular weight organic compounds such as glycerol palmitate or ethyl lactate, polymers such as poly(ethylene glycol) or poly(lactide-co-caprolactone), and other like substances. Compositions formulated with a crystallization-controlling agent include about 40-95 wt-% of the polymer, preferably about 60-90 wt-%, and about 5-60 wt-% of the crystallization-controlling agent, preferably about 10-40 wt-%.

Crystallization-controlling agents suitable for use in the present compositions may be divided into two major classes, those that persist in the form of a solid particulate in the molten composition, and those that melt or dissolve in the molten polymer composition.

Crystallization-controlling agents that will persist as solid particles, or fillers, in the composition include inorganic or organic salts, and polymers. Suitable inorganic salts include, for example, calcium carbonate, hydroxy apatite, calcium phosphate, calcium apatite, calcium sulfate, calcium bicarbonate, calcium chloride, sodium carbonate, sodium bicarbonate, sodium chloride, and other like salts. Suitable organic salts include for example, calcium stearate, calcium palmitate, sodium stearate, other metallic salts of C₁₀-C₅₀ fatty acid derivatives, and other like salts. Polymers suitable for use in the composition that persist as dispersed particles or fillers in the composition include, for example, polysaccharides, cellulose derivatives and poly(vinyl alcohol). Examples of suitable polysaccharides include, for example, dextran, maltodextrin, starches derived from corn, wheat, rice and the like, and starch derivatives such as sodium starch glycolate. Examples of suitable cellulose derivatives include for example, sodium carboxymethyl cellulose, crosslinked sodium carboxymethyl cellulose, carboxyl methyl cellulose, hydroxyethyl cellulose, and the like. Suitable poly(vinyl alcohol)s have a molecular weight of about 5,000 to 20,000, preferably about 10,000-15,000, with a percent hydrolysis of about 80-100%.

Crystallization-controlling agents which either melt with or dissolve into the molten polymer during compounding may also be used in the polymer compositions of the invention. These compositions may or may not undergo some degree of phase separation during cooling. Crystallization-controlling agents of this type include low molecular weight organic compounds and polymers. Suitable low molecular weight compounds include, for example, glycerol, palmitate, glycerol stearate and other like glycerol derivatives, triethyl citrate and other like citric acid derivatives, ethyl lactate and other like esters, and the like.

The crystallization-controlling agent is included in the composition in an amount effective to soften the polymer to a moldable and/or smearable consistency. Preferably, the crystallization-controlling agent is a non-solvent, solid substance. A crystallization-controlling agent may be included in the composition alone or in combination with another crystallization-controlling agent. An example of a preferred combination of such agents is poly(lactide-co-caprolactone) and calcium stearate.

Penetration Enhancer

The composition may further comprise a penetration enhancer effective to improve the penetration of the biological agent into and through bodily tissue, with respect to a composition lacking the penetration enhancer. The penetration enhancer may generally be any penetration enhancer, preferably is oleic acid, oleyl alcohol, ethoxydiglycol, laurocapram, alkanecarboxylic acids, dimethylsulfoxide, polar lipids, or N-methyl-2-pyrrolidone, and more preferably is oleic acid or oleyl alcohol. The penetration enhancer can be present in the flowable composition in any suitable and appropriate amount (e.g., between about 1 wt. % and about 10 wt. %)

Absorption Altering Agent

Any suitable and appropriate absorption altering agent can be employed in the present invention. For example, the absorption altering agent can be selected from the group of propylene glycol, glycerol, urea, diethyl sebecate sodium, lauryl sulfate, sodium lauryl sulfate, sorbitan ethoxylates, oleic acid, pyrrolidone carboxylate esters, N-methylpyrrolidone, N,N-diethyl-m-tolumide, dimethyl sulfoxide, alkyl methyl sulfoxides, and combinations thereof.

Opacification Agent

Any suitable and appropriate opacification agent can be employed in the present invention. For example, the opacification agent can be selected from the group of barium, iodine, calcium, and any combination thereof.

Colorant

Colorants can also be added to the liquid composition in an amount effective to allow monitoring of the biodegradability or bioerodibility of the microporous film over time. Suitable and appropriate colorants will be nontoxic, non-irritating and non-reactive with the solvent in the liquid composition. Colorants which have been approved by the FDA for use in cosmetics, foods and drugs include: D & C Yellow No. 7; D & C Red No. 17; D & C Red No. 7, 9, and 34; FD & C Red No. 4; Orange D & C No. 4; FD & C Blue 2; FD & C Green No. 3, and the like.

Moldable Implant Precursor

The flowable composition can be formed into a moldable implant precursor by its contact with an aqueous medium such as water or saline, or contact with a body fluid such as blood serum, lymph, and the like pursuant to the techniques disclosed in U.S. Pat. No. 5,487,897, the disclosure of which is incorporated herein by reference with the specification that the thermoplastic polymer of the '897 patent is a biocompatible, biodegradable, thermoplastic polymer as described herein.

Briefly, the technique disclosed by the '897 patent converts the flowable composition with or without bioactive agent into a two-part structure comprising an outer sac with a flowable content. The technique applies a limited amount of aqueous medium and the like to a quantity of the pharmaceutical system so that only the outer surface of the system is converted to solid, thus forming the sac with a flowable content inside. The flowable content of the implant precursor may range in consistency from watery to viscous. The outer sac may range in consistency from gelatinous to an impressionable, moldable and waxen-like. The resulting device, or implant precursor, may then be applied to an implant site. Upon implantation, the solvent from the implant precursor diffuses into the surrounding tissue fluids to form an implant having a solid polymer matrix. Preferably, the implant precursor solidifies in situ to a solid matrix within about 0.5-4 hours after implantation, preferably within about 1-3 hours, preferably within about 2 hours. Thus, when placed into an implant site in a body, the implant precursor eventually coagulates to a solid, microporous matrix structure.

Porous Structure

The porous structure of the solid matrices, e.g., in situ formed implants, implants, implantable articles, biodegradable articles and devices of the invention, is influenced by nature of the organic solvent and thermoplastic polymer, by their solubility in water, aqueous medium or body fluid (which may differ for each medium) and by the presence of an additional substances (e.g., pore forming moiety). The porous structure is believed to be formed by several mechanisms and their combinations. The dissipation, disbursement or diffusion of the solvent out of the solidifying flowable composition into the adjacent fluids may generate pores, including pore channels, within the polymer matrix. The infusion of aqueous medium, water or body fluid into the flowable composition also occurs and is in part also responsible for creation of pores. Generally, it is believed that the porous structure is formed during the transformation of the flowable composition to an implant, article and the like. During this process, it is believed, as explained above, that the organic solvent and thermoplastic polymer partition within the flowable composition into regions that are rich and poor in thermoplastic polymer. The partition is believed to occur as a result of the dynamic interaction of aqueous infusion and solvent dissipation. The infusion involves movement of aqueous medium, water or body fluid into the flowable composition and the dissipation involves movement of the organic solvent into the medium surrounding the flowable composition. The regions of the flowable composition that are poor in thermoplastic polymer become infused with a mixture of organic solvent and water, aqueous medium or body fluid. These regions are believed to eventually become the porous network of the implant, article and the like.

Typically, the macroscopic structure of the solid matrix involves a core and a skin. Typically, the core and skin are microporous but the skin pores are of smaller size than those of the core unless a separate pore forming agent is used as discussed below. Preferably, the outer skin portion of the solid matrix has pores with diameters significantly smaller in size than these pores in the inner core portion. The pores of the core are preferably substantially uniform and the skin is typically functionally non-porous compared to the porous nature of the core. The size of the pores of the implant, article, device and the like are in the range of about 4-1000 microns, preferably the size of pores of the skin layer are about 1-500 microns. The porosity of such matrices is described by U.S. Pat. No. 5,324,519, the disclosure of which is incorporated herein by reference.

The solid microporous implant, article, device and the like will have a porosity in the range of about 5-95% as measured by the percent solid of the volume of the solid. The development of the degree of porosity will be governed at least in part by the degree of water solubility of the organic solvent and thermoplastic polymer. If the water solubility of the organic solvent is high and that of the polymer is extremely low or non-existent, a substantial degree of porosity will be developed, typically on the order of 30 to 95%. If the organic solvent has a low water solubility and the polymer has a low to non-existent water solubility, a low degree of porosity will be developed, typically on the order of 5 to 40%. It is believed that the degree of porosity is in part controlled by the polymer-solvent partition when the flowable composition contacts an aqueous medium and the like. The control of the degree of porosity is beneficial for generation of differing kinds of biodegradable articles, implants and devices according to the invention. For example, if strength is a requirement for the article, implant or device and the like, it may be beneficial to have a low degree of porosity.

Solid Biodegradable Articles

Biodegradable drug delivery products can be prepared by the transformation process using water or an aqueous medium or body fluid to cause solidification. Generally, these products are ex vivo solid matrices. If the ex vivo solid matrix is to have a particular shape, it can be obtained by transforming the flowable composition in a suitable mold following the moldable implant precursor technique described above. After the precursor has been formed, it can be contacted with additional aqueous medium to complete the transformation. Alternatively, the flowable composition can be placed in a closed mold that is permeable to aqueous medium and the mold with composition can be contacted with aqueous medium such as be submerging in an aqueous bath. Preferably, the flowable composition in this instance will have a moderate to high viscosity.

Microcapsules and microparticles can be formed by techniques known in the art. Briefly, the microcapsule preparation involves formation of an emulsion of bioactive agent-carrier micelles in the flowable composition where the carrier is a nonsolvent for the biocompatible, biodegradable, branched thermoplastic polymer of the invention. The micelles are filtered and then suspended in an aqueous medium. The coating of flowable composition on the surfaces of the micelles then solidifies to form the porous microcapsules. Microparticles are formed in a similar process. A mixture of flowable composition and bioactive agent is added dropwise by spraying, dripping, aerosolizing or by other similar techniques to a nonsolvent for the flowable composition. The size and shape of the droplets is controlled to produce the desired shape and size of the porous microparticles. Sheets, membranes and films can be produced by casting the flowable composition onto a suitable nonsolvent and allowing the transformation to take place. Similarly, the viscosity of the flowable composition can be adjusted so that when sprayed or aerosolized, strings rather than droplets are formed. These strings can be cast upon a nonsolvent for the flowable composition such that a filamentous scaffold or membrane is produced. Also, suture material or other similar material can be formed by extrusion of the flowable composition into a non-solvent bath. The extrusion orifice will control the size and shape of the extruded product. The techniques for formation of these ex vivo solid matrices are described in U.S. Pat. Nos. 4,652,441; 4,917,893; 4,954,298; 5,061,492; 5,330,767; 5,476,663; 5,575,987; 5,480,656; 5,643,607; 5,631,020; 5,631,021; 5,651,990, the disclosures of which are incorporated herein by reference with the proviso that the polymers used are the biocompatible, biodegradable, thermoplastic polymers disclosed herein.

These ex vivo solid matrices can be used according to their known functions. Additionally, the implants and other solid articles are can be inserted in a body using techniques known to the art such as through an incision or by trocar.

The present invention also provides an implant. The implant includes a biodegradable, biocompatible thermoplastic polymer that is at least substantially insoluble in aqueous medium, water or body fluid; and a cell-cycle dependent biological agent, a schedule-dependent biological agent, a metabolite thereof, a pharmaceutically acceptable salt thereof, or a prodrug thereof. The implant has a solid or gelatinous microporous matrix, wherein the matrix is a core surrounded by a skin. The implant can further include a biocompatible organic liquid, at standard temperature and pressure, in which the thermoplastic polymer is soluble. The amount of biocompatible organic liquid, if present, is preferably minor, such as from about 0 wt. % to about 20 wt. % of the composition. In addition, the amount of biocompatible organic liquid preferably decreases over time. The core preferably contains pores of diameters from about 1 to about 1000 microns. The skin preferably contains pores of smaller diameters than those of the core pores. In addition, the skin pores are preferably of a size such that the skin is functionally non-porous in comparison with the core. The implant can have any suitabke shape and can have any suitable form. For example, the implant can be a solid, semi-solid, wax-like, viscous, or the implant can be gelatinous.

Cancer Treatment

The flowable composition can be employed to treat cancer in a mammal. Specifically, the mammal can be a human. Additionally, the cancer can be a tumor, such as a solid tumor. Tumors treatable with the compositions and methods of the present invention can be located in any part of the mammal. Specifically, the tumor (e.g., solid tumor) can be located in the breast, lung, thyroid, lymph node, genitourinary system, kidney, ureter, bladder, ovary, testis, prostate, musculoskeletal system, bone, skeletal muscle, bone marrow, gastrointestinal tract, stomach, esophagus, small bowel, colon, rectum, pancreas, liver, smooth muscle, central or peripheral nervous system, brain, spinal cord, nerves, head, neck, ear, eye, nasopharynx, oropharynx, salivary gland, cardiovascular system, oral cavity, tongue, larynx, hypopharynx, soft tissues, skin, cervix, anus, retina, and/or heart.

As used herein, “treating” or “treat” includes (i) preventing a pathologic condition (e.g., a solid tumor) from occurring (e.g. prophylaxis); (ii) inhibiting the pathologic condition (e.g., a solid tumor) or arresting its development; and (iii) relieving the pathologic condition (e.g., relieving the symptoms associated with a solid tumor).

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

“Therapeutically effective amount” is intended to include an amount of a chemotherapeutic compound useful in the present invention or an amount of the combination of chemotherapeutic compounds, e.g., to treat or prevent a solid tumor or to treat the symptoms associated with a solid tumor in a host. The combination of chemotherapeutic compounds is preferably a synergistic combination. Synergy, as described for example by Chou and Talalay, Adv. Enzyme Regul. 22:27-55 (1984), occurs when the effect (in this case, treatment or prevention of cancer) of the chemotherapeutic compounds when administered in combination is greater than the additive effect of the chemotherapeutic compounds when administered alone as a single agent. In general, a synergistic effect is most clearly demonstrated at suboptimal concentrations of the chemotherapeutic compounds. Synergy can be in terms of lower cytotoxicity, increased activity, or some other beneficial effect of the combination compared with the individual components.

As used herein, “pharmaceutically acceptable salts” refer to derivatives (e.g., of the chemotherapeutic agents) wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, isethionic, and the like. Specifically, the pharmaceutically acceptable salts can include those salts that naturally occur in vivo in a mammal.

The pharmaceutically acceptable salts (e.g., of the chemotherapeutic agents) useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds (e.g., chemotherapeutic agents) which are, within the scope of sound medical judgment, suitable for use in contact with the tissues of human beings and animals without excessive toxicity, irritation, allergic response, or other problem or complication commensurate with a reasonable benefit/risk ratio.

Pharmaceutical Kits

The present invention provides pharmaceutical kits. Such kits are suitable for in situ formation of a biodegradable implant in a body. The kits can include a first container that includes a flowable composition. The composition can include a biodegradable, biocompatible thermoplastic polymer that is at least substantially insoluble in aqueous medium, water or body fluid; and a biocompatible organic liquid at standard temperature and pressure, in which the thermoplastic polymer is soluble. The kit can also include a second container that includes a cell-cycle dependent biological agent, a schedule-dependent biological agent, a metabolite thereof, a pharmaceutically acceptable salt thereof, or a prodrug thereof. The pharmaceutical kit can further optionally include instructions or printed indicia for assembling and/or using the pharmaceutical kit.

Specifically, the first container can include a syringe or a catheter; and the second container can independently include a syringe or a catheter. Additionally, the first container can include a syringe, the second container can include a syringe, and both syringes can be configured to directly connect to each other.

Specific Ranges, Values, and Embodiments

In one specific embodiment of the present invention, the biodegradable, biocompatible thermoplastic polymer can have a formula incorporating monomeric units selected from the group of lactides, glycolides, caprolactones, glycerides, anhydrides, amides, urethanes, esteramides, orthoesters, dioxanones, acetals, ketals, carbonates, phosphazenes, hydroxybutyrates, hydroxyvalerates, alkylene oxalates, alkylene succinates, amino acids, and any combination thereof, and the formula contains the monomeric units random or block order.

In another specific embodiment of the present invention, the biodegradable, biocompatible thermoplastic polymer can be a polymer or copolymer of lactide monomeric units, caprolactone monomeric units, glycolide monomeric units, or any combination thereof.

In another specific embodiment of the present invention, the biodegradable, biocompatible thermoplastic polymer can include a polymer selected from the group of polylactides, polyglycolides, polycaprolactones, polydioxanones, polycarbonates, polyhydroxybutyrates, polyalkyene oxalates, polyanhydrides, polyamides, polyesteramides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, poly(methyl vinyl ether), polyesters, polyalkylglycols, copolymers thereof, block copolymers thereof, terpolymers thereof, combinations thereof, and mixtures thereof.

In another specific embodiment of the present invention, the biodegradable, biocompatible thermoplastic polymer can include at least one polyester.

In another specific embodiment of the present invention, the biodegradable, biocompatible thermoplastic polymer can be at least one of a polylactide, a polyglycolide, a polycaprolactone, a copolymer thereof, a terpolymer thereof, or any combination thereof.

In another specific embodiment of the present invention, the biodegradable, biocompatible thermoplastic polymer can be a poly (DL-lactide-co-glycolide). In another specific embodiment of the present invention, the biodegradable, biocompatible thermoplastic polymer can be a poly (DL-lactide-co-glycolide) having a carboxy terminal group. In another specific embodiment of the present invention, the biodegradable, biocompatible thermoplastic polymer can be a poly (DL-lactide-co-glycolide) without a carboxy terminal group. In another specific embodiment of the present invention, the biodegradable, biocompatible thermoplastic polymer can be 50/50 poly (DL-lactide-co-glycolide) having a carboxy terminal group. In another specific embodiment of the present invention, the biodegradable, biocompatible thermoplastic polymer can be 75/25 poly (DL-lactide-co-glycolide) without a carboxy terminal group.

In another specific embodiment of the present invention, the biodegradable, biocompatible thermoplastic polymer can be present in up to about 80 wt. % of the composition. In another specific embodiment of the present invention, the biodegradable, biocompatible thermoplastic polymer can be present in more than about 10 wt. % of the composition. In another specific embodiment of the present invention, the biodegradable, biocompatible thermoplastic polymer can be present in about 10 wt. % to about 80 wt. % of the composition. In another specific embodiment of the present invention, the biodegradable, biocompatible thermoplastic polymer can be present in about 30 wt. % to about 50 wt. % of the composition.

In another specific embodiment of the present invention, the biodegradable, biocompatible thermoplastic polymer can have an average molecular weight of more than about 15,000. In another specific embodiment of the present invention, the biodegradable, biocompatible thermoplastic polymer can have an average molecular weight of up to about 45,000. In another specific embodiment of the present invention, the biodegradable, biocompatible thermoplastic polymer can have an average molecular weight of about 15,000 to about 45,000.

In one embodiment of the present invention, the biocompatible organic liquid can have a water solubility ranging from completely insoluble in any proportion to completely soluble in all proportions. In another embodiment of the present invention, the biocompatible organic liquid can be completely insoluble in water but will diffuse into body fluid. In another embodiment of the present invention, the biocompatible organic liquid can be at least partially water-soluble. In another embodiment of the present invention, the biocompatible organic liquid can be completely water-soluble. In another embodiment of the present invention, the biocompatible liquid can be dispersible in aqueous medium, water, or body fluid.

In another embodiment of the present invention, the biocompatible organic liquid can be a polar protic liquid. In another embodiment of the present invention, the biocompatible organic liquid can be a polar aprotic liquid.

In another embodiment of the present invention, the biocompatible organic liquid can be a cyclic, aliphatic, linear aliphatic, branched aliphatic or aromatic organic compound, that is liquid at ambient and physiological temperature, and contains at least one functional group selected from the group of alcohols, ketones, ethers, amides, amines, alkylamines, esters, carbonates, sulfoxides, sulfones, and sulfonates.

In another embodiment of the present invention, the biocompatible organic liquid can be selected from the group of substituted heterocyclic compounds, esters of carbonic acid and alkyl alcohols, alkyl esters of monocarboxylic acids, aryl esters of monocarboxylic acids, aralkyl esters of monocarboxylic acids, alkyl esters of dicarboxylic acids, aryl esters of dicarboxylic acids, aralkyl esters of dicarboxylic acids, alkyl esters of tricarboxylic acids, aryl esters of tricarboxylic acids, aralkyl esters of tricarboxylic acids, alkyl ketones, aryl ketones, aralkyl ketones, alcohols, polyalcohols, alkylamides, dialkylamides, alkylsulfoxides, dialkylsulfoxides, alkylsulfones, dialkylsulfones, lactones, cyclic alkyl amides, cyclic alkyl amines, aromatic amides, aromatic amines, mixtures thereof, and combinations thereof.

In another embodiment of the present invention, the biocompatible organic liquid can be selected from the group of N-methyl-2-pyrrolidone, 2-pyrrolidone, (C₂-C₈) aliphatic alcohol, glycerol, tetraglycol, glycerol formal, 2,2-dimethyl-1,3-dioxolone-4-methanol, ethyl acetate, ethyl lactate, ethyl butyrate, dibutyl malonate, tributyl citrate, tri-n-hexyl acetylcitrate, diethyl succinate, diethyl glutarate, diethyl malonate, triethyl citrate, triacetin, tributyrin, diethyl carbonate, propylene carbonate, acetone, methyl ethyl ketone, dimethylacetamide, dimethylformamide, caprolactam, dimethyl sulfoxide, dimethyl sulfone, tetrahydrofuran, caprolactam, N,N-diethyl-m-toluamide, 1-dodecylazacycloheptan-2-one, 1,3-dimethyl-3,4,5,6-tetrahydro-2-(1H)-pyrimidinone, benzyl benzoate, and combinations thereof.

In another embodiment of the present invention, the biocompatible organic liquid can have a molecular weight in the range of about 30 to about 500.

In another embodiment of the present invention, the biocompatible organic liquid can be N-methyl-2-pyrrolidone, 2-pyrrolidone, N,N-dimethylformamide, dimethyl sulfoxide, propylene carbonate, caprolactam, triacetin, or any combination thereof. In another embodiment of the present invention, the biocompatible organic liquid can be N-methyl-2-pyrrolidone.

In another embodiment of the present invention, the biocompatible liquid can be present in more than about 40 wt. % of the composition. In another embodiment of the present invention, the biocompatible liquid can be present in up to about 80 wt. % of the composition. In another embodiment of the present invention, the biocompatible liquid can be present in about 50 wt. % to about 70 wt. % of the composition.

In one embodiment of the present invention, the cell-cycle dependent biological agent or schedule-dependant biological agent can be a compound that blocks, impedes, or otherwise interferes with, cell cycle progression at the G1-phase, G1/S interface, S-phase, G2/M interface, or M-phase of the cell cycle; or is a metabolite or prodrug thereof.

In another embodiment of the present invention, the cell-cycle dependent biological agent or schedule-dependant biological agent can be an analogue of a uridine nucleoside, an analogue of a thymidine nucleoside, an analogue of a uridine nucleoside, or an analogue of a thymidine nucleoside; a modulator of a fluoropyrimidine; a cytidine analogue or a cytidine nucleoside analogue; a purine analogue or a purine nucleoside analogue; an antifolate; an antimetabolite; an S-phase specific radiotoxin (deoxythymidine analogue); an inhibitor of an enzyme involved in deoxynucleoside/deoxynucleotide metabolism; a DNA chain-terminating nucleoside analogue; an inhibitor of an enzyme that regulates, directly or indirectly, cell cycle progression through the G1-phase, G1/S interface or S-phase of the cell cycle; a cytokine, growth factor, anti-angiogenic factor or other protein that inhibits cell cycle progression at the G1-phase or G1/S interface of the cell cycle; a drug or compound that inhibits cell cycle progression at the G2/M interface, or M-phase of the cell cycle; a taxane microtubule-targeting drug; a vinca alkaloid microtubule-targeting drug; another microtubule-targeting drug; an inhibitor of serine-threonine kinase, that regulate progression through the G2/M interface or M-phase of the cell cycle; or a metabolite or prodrug thereof.

In another embodiment of the present invention, the analogue of a uridine nucleoside, analogue of a thymidine nucleoside, analogue of a uridine nucleoside, analogue of a thymidine nucleoside, metabolite thereof, or prodrug thereof, can be 5-fluorodeoxyuridine (floxuridine, FUDR), 5-Flurouracil (5-FU), a prodrug of 5-FU, bromodeoxyuridine, iododexoyuridine, or a prodrug of halopyrimidine. In another embodiment of the present invention, the prodrug of 5-FU can be capecitabine, 5′-deoxy-5-fluorouridine, ftorafur, or flucytosine. In another embodiment of the present invention, the prodrug of halopyrimidine can be a polymeric prodrug of halopyrimidine.

In another embodiment of the present invention, the modulator of a fluoropyrimidine can be leurovorin, methotrexate, levamisole, acivicin, phosphonacetyl-L-aspartic acid (PALA), brequinar, or 5-ethynyluracil uracil.

In another embodiment of the present invention, the cytidine analogue, cytidine nucleoside analogue, metabolite or prodrug thereof, can be cytarabine (Ara-C, cytosine arabinoside), Gemcitabine (2′,2′-difluorodeoxycytidine), 5-azacytidine, or a prodrug of a cytidine analogue. In another embodiment of the present invention, the prodrug of a cytidine analogue can be a polymeric prodrug of a cytidine analogue.

In another embodiment of the present invention, the purine analogue, purine nucleoside analogue, metabolite thereof or prodrug thereof, can be 6-thioguanine, 6-mercaptopurine, azathioprine, adenosine arabinoside (Ara-A), 2′,2′-difluorodeoxyguanosine, deoxycoformycin (pentostatin), cladribine (2-chlorodeoxyadenosine), an inhibitor of adenosine deaminase, or a prodrug of a purine analogue. In another embodiment of the present invention, the prodrug of a purine analogue can be a polymeric prodrug of a purine analogue.

In another embodiment of the present invention, the antifolate, metabolite thereof, or prodrug thereof, can be methotrexate, aminopterin, trimetrexate, edatrexate, N10-propargyl-5,8-dideazafolic acid (CB3717), ZD 1694, 5,8-dideazaisofolic acid (IAHQ), 5,10-dideazatetrahydrofolic acid (DDATHF), 5-deazafolic acid (efficient substrate for FPGS), PT523 (N alpha-(4-amino-4-deoxypteroyl)-N delta-hemiphthaloyl-L-ornithine), 10-ethyl-10-deazaaminopterin (DDATHF, lomatrexol), piritrexim, 10-EDAM, ZD1694, GW1843, PDX (10-propargyl-10-deazaaminopterin), multi-targeted folate, a folate-based inhibitor of thymidylate synthase (TS), a folate-based inhibitor of dihydrofolate reductase (DHFR), a folate-based inhibitor of glycinamide ribonucleotide transformylase (GARTF), an inhibitor of folylpolyglutamate synthetase (FPGS), a folate-based inhibitor of GAR formyl transferase (AICAR transformylase).

In another embodiment of the present invention, the multi-targeted folate can be LY231514 or permetrexed. In another embodiment of the present invention, the antimetabolite can be hydroxyurea or a polyamine. In another embodiment of the present invention, the S-phase specific radiotoxin (deoxythymidine analogue) can be [¹²⁵I]-iododeoxyuridine, [¹²³I]-iododeoxyuridine, [¹²⁴I]-iododeoxyuridine, [^{80m}Br]-iododeoxyuridine, [¹³¹I]-iododeoxyuridine, or [²¹¹At]-astatine-deoxyuridine.

In another embodiment of the present invention, the inhibitor of an enzyme involved in deoxynucleoside/deoxynucleotide metabolism can be an inhibitor of thymidylate synthase (TS), an inhibitor of dihydrofolate reductase (DHFR), an inhibitor of glycinamide ribonucleotide transformylase (GARTF), an inhibitor of folylpolyglutamate synthetase (FPGS), an inhibitor of GAR formyl transferase (AICAR transformylase), an inhibitor of DNA Polymerase (DNA Pol), an inhibitor of ribonucleotide reductase (RNR), an inhibitor of thymidine kinase (TK), or an inhibitor of topoisomerase I enzymes.

In another embodiment of the present invention, the inhibitor of DNA Polymerase can be Aphidocolin. In another embodiment of the present invention, the inhibitor of topoisomerase I enzymes can be camptothecins, irinotecan [CPT-11, camptosar], topotecan, NX-211 [lurtotecan] or rubitecan. In another embodiment of the present invention, the DNA chain-terminating nucleoside analogue can be acyclovir, abacavir, valacyclovir, zidovudine (AZT), didanosine (ddI, dideoxycytidine), zalcitabine (ddC), stavudine D4T), lamivudine (3TC), a 2′3′-dideoxy nucleoside analogue, or a 2′3′-dideoxy nucleoside analogue that terminates DNA synthesis.

In another embodiment of the present invention, the inhibitor of an enzyme that regulates, directly or indirectly, cell cycle progression through the G1-phase, G1/S interface or S-phase of the cell cycle can be an inhibitor of growth factor receptor tyrosine kinases that regulates progression through the G1-phase, G1/S interface, or S-phase of the cell cycle, an inhibitor of non-receptor tyrosine kinases, an inhibitor of serine-threonine kinases that regulate progression through the G1-phase, G1/S interface or S-phase of the cell cycle, an inhibitor of G-proteins and cGMP phosphodiesterases that positively regulate cell cycle progression at the G1-phase, G1/S interface or S-phase of the cell cycle, a drug that inhibits the induction of immediate early response transcription factors, or a drug that inhibits proteosomes that degrade negative cell cycle regulatory compounds.

In another embodiment of the present invention, the inhibitor of growth factor receptor tyrosine kinases that regulates progression through the G1-phase, G1/S interface, or S-phase of the cell cycle can be trastusumab, iressa, erbitux, or tarceva. In another embodiment of the present invention, the inhibitor of non-receptor tyrosine kinase can be gleevec. In another embodiment of the present invention, the cytokine, growth factor, anti-angiogenic factor or other protein that inhibits cell cycle progression at the G1-phase or G1/S interface of the cell cycle can be an interferon, interleukin, somatostatin, a somatostatin analogue, or an anti-angiogenic factor that inhibits cell proliferation of endothelial cells at the G1 or G1/S phases of the cell cycle.

In another embodiment of the present invention, the somatostatin or somatostatin analogue can be octreotide or sandostatin LAR. In another embodiment of the present invention, the microtubule-targeting drug can be taxol, taxotere, epothilones, a taxane derivative, vinca alkaloid, vinblastine, vincristine, vindesine, vinflunine, vinorelbine, vinzolidine, nocadazole, colchicine, estramustine or CP-461.

In another embodiment of the present invention, the inhibitor of serine-threonine kinase, that regulates progression through the G2/M interface or M-phase of the cell cycle, can be an inhibitor of G2/M cyclin-dependent kinase, an inhibitor of M-phase cyclin, or a drug that blocks, impedes, or otherwise interferes with, cell cycle progression at the G2/M interface, or M-phase of the cell cycle.

In another embodiment of the present invention, the cell-cycle biological agent, schedule-dependant biological agent, metabolite thereof, pharmaceutically acceptable salt thereof, or prodrug thereof can be present in more than about 0.00001 wt. % of the composition. In another embodiment of the present invention, the cell-cycle biological agent, schedule-dependant biological agent, metabolite thereof, pharmaceutically acceptable salt thereof, or prodrug thereof can be present in up to about 20 wt. % of the composition. In another embodiment of the present invention, the cell-cycle biological agent, schedule-dependant biological agent, metabolite thereof, pharmaceutically acceptable salt thereof, or prodrug thereof can be present in about 0.00001 wt. % to about 10 wt. % of the composition.

In another embodiment of the present invention, the human maximum tolerated dose (MTD) of the cell-cycle biological agent, schedule-dependant biological agent, metabolite thereof, or prodrug thereof, present in the flowable composition can be less than the human maximum tolerated dose (MTD) of the cell-cycle biological agent, schedule-dependant biological agent, metabolite thereof, or prodrug thereof, present in solution (i.e., another carrier). In another embodiment of the present invention, the human maximum tolerated dose (MTD) of the cell-cycle biological agent, schedule-dependant biological agent, metabolite thereof, or prodrug thereof, present in the flowable composition can be at least 50% less than the human maximum tolerated dose (MTD) of the cell-cycle biological agent, schedule-dependant biological agent, metabolite thereof, or prodrug thereof, present in solution (i.e., another carrier).

In one specific embodiment of the present invention, the second chemotherapeutic agent can act at various stages of the cell cycle. In another specific embodiment of the present invention, the second chemotherapeutic agent can be an antracycline (e.g., doxorubicin, daunorubicin, epirubicin, idarubicin, or mitoxantrone); a DNA intercalator (e.g., actinomycin C, actinomycin D, actinomycin B, a podophyllotoxin, or an epipodophyllatoxin such as an etoposide, teniposide, or ctoposide); an alkylating agent (e.g., mechlorethamine, melphalan, cyclophosphamide, chlorambucil, ifosfamide, carmustine, lomustine, busulfan, dacarbazine, cisplatin, carboplatin, oxaliplatin, iproplatin, or tetraplatin); a hormonal agent (e.g., an antiestrogen/estrogen antagonist, an LHRH agonist or antagonist such as leuprolide acetate, goserelin, or abarelix; an aromatase inhibitor, or an antiandrogen); a chemoprevention agent, a metabolite thereof, or a prodrug thereof. In another specific embodiment of the present invention, the second chemotherapeutic agent can be an NSAID or cis-retinoid.

Additional suitable polymers, solvents, additives, and chemotherapeutic agents are described in U.S. Provisional Patent Application Ser. No. 60/454,100, filed on Mar. 11, 2003, and/or U.S. Provisional Patent Application Ser. No. 60/505,124, filed on Sep. 22, 2003, which applications are herein incorporated by reference.

### EXAMPLES

Atrix Laboratories investigated the use of 2-deox-5-fluorouridine (Floxuridine) in their Atrige® delivery system as a locally-delivered cancer chemotherapeutic agent. Floxuridine (FUDR) is currently marketed for use in treatment of metastatic carcinoma. The mechanism of action of FUDR involves a complex metabolic pathway leading to production of a metabolite that inhibits an intracellular enzyme [thymidylate synthase (TS)] critical to the DNA repair process and promotes incorporation of this metabolite into DNA. There is also inhibition of thymidylate monophosphate (dTMP), a precursor of thymidine triphosphate (dTTP) a substrate for DNA synthesis. It is thought that incorporation of the metabolite into DNA causes strand breaks by excision followed by inhibition of the repair process leading to cell death. The recommended therapeutic dose in humans is via continuous intra-arterial infusion (intrahepatic artery or into arterial blood supplies of tumors) at 0.6 mg/kg/day for up to 14 days.

Animal toxicology - Lethal
Intravenous Doses of FUDR¹

| Species | ( [symbol] ) |
|---|---|
| Mouse | 880 +/− 51 |
| Rat | 670 +/− 73 |
| Rabbit | 94 +/− 19.6 |
| Dog | 157 +/− 46 |

Proposed injection doses (volume & quantity) of
Atrigel ® -Floxuridine Compared to Current
Recommended Therapeutic doses in humans

| [symbol] | [symbol] | [symbol] | [symbol] | [symbol] |
|---|---|---|---|---|
| 10 | 1 | 0.143 | 0.024 | 0.010 |
| 20 | 2 | 0.286 | 0.048 | 0.020 |
| 50 | 5 | 0.714 | 0.119 | 0.050 |
| 100 | 10 | 1.428 | 0.238 | 0.102 |

| [symbol] | [symbol] | [symbol] | [symbol] | [symbol] |
|---|---|---|---|---|
| 10 | 1 | 40 (0.3) | 0.045 | 0.0035 |
| 20 | 2 | 80 (0.7) | 0.090 | 0.007 |
| 50 | 5 | 200 (1.7) | 0.227 | 0.018 |
| 100 | 10 | 400 (3.3) | 0.454 | 0.035 |

Assumes 70 kg individual
Maximum Daily Dose: 0.6 mg/kg/day × 70 kg = 42 mg/day
Minimum Daily Dose: 0.1 mg/kg/day × 70 kg = 7 mg/day
Mininum Total Dose: 0.1 mg/kg/day × 70 kg × 14 days = 98 mg

The Atrigel® delivery system has been shown to be safe and effective in laboratory animals (rodents and non-rodents) used in regulatory toxicology studies to support clinical trials and in humans in clinical trials. This delivery system is utilized in many currently FDA-approved human pharmaceutical products including Atridox® and Eligard® 1-, 3-, and 4-month formulations. Administration is via the subcutaneous route with constant release of drug over periods up to 4 months after a single injection.

Numerous studies in rats have been performed that demonstrate the sustained release of FUDR from Atrigel® following single subcutaneous injection of doses up to 2 mg/kg in a dose volume of 50 μL. In these studies, there were minimal side-effects including transient minimal body weight decrease and body weight loss and minimal to marked injection site reactions (erythema, edema, vasodilation). The ATRIGEL® Floxuridine formulation was also evaluated in non-tumor bearing and tumor bearing immuno-incompetent SCID mice. Doses were administered by intraperitoneal (i.p.), intratumoral (i.t.), and subcutaneous (s.c.) routes. FUDR was administered as “free” (up to 150 mg/kg i.p. solution in saline) or as an ATRIGEL® Floxuridine formulation (10% FUDR w/v). ATRIGEL® alone and FUDR in saline solution were administered without adverse effects on body weights or survival. The ATRIGEL® Floxuridine formulation administered at different doses (up to 150 mg/kg×5 s.c. or ranging from 50 to 100 mg/kg×1 i.t.), volumes (10 to 20 μL), and schedules (q.d.×5, ×1, ×2 on Days 1 and 14), caused mortality but showed some activity in slowing tumor growth.

Reported Toxicities With FUDR (Physicians Desk Reference, 57 Ed., 2003; Casarett & Doulls TOXICOLOGY, 6th Ed., 2001). In laboratory animals: Bone Marrow; Teratogenic, mouse at 2.5 and 100 mg/kg, rat at 75 and 150 mg/kg (cleft palate, skeletal defects, limb deformities); Reproductive Toxicant, spermato-toxic in rats at 125-250 mg/kg i.p., reproductive toxicity in female rats at 25 or 50 mg/kg; Cardiotoxicity, arrhythema; Vasculotoxic; Allergen; Ovarian Toxicity; Hematotoxic (leukopenia); Mutagenic in mouse embryo fibroblasts. Human adverse reactions: Gastrointestinal—ulcers, bleeding; Dermatological—lopecia, dermatitis; Cardiovascular—myocardial ischemia.

The Examples below demonstrate the feasibility and efficacy potential for local (intratumoral) delivery of Floxuridine in the Atrigel® delivery system to an animal tumor model. By delivering Floxuridine (FUDR) in a time-released format, a higher concentration should have been able to be administered without the toxic effects associated with the delivery of the free drug. The outcome of these studies was opposite of that hypothesis, as ATRIGEL®-FUDR has a lower maximum tolerated dose (MTD) than that of FUDR delivered as a free drug.

In these studies in tumor bearing mice models, Floxuridine delivered by ATRIGEL® was able to decrease the rate of tumor growth by approximately 50% (indicating efficacy) compared to 1) untreated controls, 2) tumor bearing mice treated with ATRIGEL® alone, or 3) Floxuridine as a free drug.

### Example 1

Floxuridine Dose Determination in SCID Mice

Introduction

This example was conducted to determine the Maximum Tolerated Dose (MTD) of Floxuridine in SCID mice when delivered via intraperitoneal (i.p.) injection. In order to compare the efficacy of Floxuridine delivered via the Atrix sustained release system and free Floxuridine as anti-tumor agents, the maximum tolerated dose (MTD) of Floxuridine in SCID mice needed to be established. It was hypothesized that by delivering Floxuridine in a time-release format, a higher concentration could be administered without the toxic effects associated with delivery of the free drug. A literature search indicated that the maximum tolerated dose of Floxuridine in normal mice is 50 mg/kg/day×5 days when administered by intraperitoneal injection.

Materials and Methods

In this 4-week study in SCID mice, FUDR was delivered as a free drug suspended in sterile saline. Male mice were given Floxuridine by intraperitoneal injection, daily for a total of 5 injections, in doses of 40, 45, 50, and 55 mg/kg/day. Mice were monitored throughout the injection series and subsequent follow-up for toxicity symptoms. Each treatment group consisted of 5 SCID mice.

Treatment Groups:

Control—vehicle only

Floxuridine (i.p., QD×5):

40 mg/kg

45 mg/kg

50 mg/kg

55 mg/kg

Total: 25 mice

Schedule:

All SCID mice used for this study were screened for IgG production and “leaky” mice were eliminated from the study group.
Mice were given i.p. injections daily for 5 days with the appropriate dose of Floxuridine.
Total injection volume were 0.1 mL/mouse/injection.
Mice were monitored weekly for weight and toxicity symptoms (survival, general health, fur condition, etc.) for at least 8 weeks (7 weeks after the last injection).

Results and Discussion

In this dose determination study, Floxuridine was delivered as free drug suspended in sterile saline solution to SCID mice. A review of the literature indicated that the Maximum Tolerated Dose (MTD) in immunocompetent mice is 50 mg/kg/day×5 days. Mice received 5 daily intraperitoneal injections in doses of 40 to 55 mg/kg/day. No toxic clinical symptoms were displayed and the experiment was terminated 3 weeks after completion of the injection series. Mouse weight, percent weight change, and dose data are shown below.

[symbol]

Mice

| Dose | Dose | | Mortality | Tumor Volume | Body Weight |
|---|---|---|---|---|---|
| (mg/kg) | Volume | Route | (%) | Change | Change |
| 40 | 0.1 mL | ip | NA | NA | NA |
| FUDR/ | | | | | |
| Saline | | | | | |
| q.d. × 5 | | | | | |
| 45, | 0.1 mL | ip | NA | NA | NA |
| FUDR/ | | | | | |
| Saline | | | | | |
| q.d. × 5 | | | | | |
| 50 | 0.1 mL | ip | NA | NA | NA |
| FUDR/ | | | | | |
| Saline | | | | | |
| q.d. × 5 | | | | | |
| 55 | 0.1 mL | ip | NA | NA | NA |
| FUDR/ | | | | | |
| Saline | | | | | |
| q.d. × 5 | | | | | |

[image]

### Example 2

Floxuridine Dose Determination in SCID Mice II

Introduction

This example was conducted to determine the Maximum Tolerated Dose (MTD) of Floxuridine in SCID mice when delivered by either intraperitoneal injection of the free drug or by subcutaneous (s.c.) injection in a sustained release format.

In order to compare the efficacy of Floxuridine delivered via the Atrix sustained release system and free Floxuridine as anti-tumor agents, the maximum tolerated dose (MTD) of Floxuridine in SCID mice must be determined for each delivery format. It is hypothesized that by delivering Floxuridine in a time-release format a higher concentration can be administered without the toxic effects associated with delivery of the free drug. Although a literature search indicated that the MTD of Floxuridine in normal mice is 50 mg/kg/day×5 days, the initial dose determination experiment (Example 1) did not show toxicity in doses of 40 to 55 mg/kg/day×5 days. In this Example, the dose range is extended, consisting of 50, 75, 100 or 150 mg/kg/day×5 days. Identical doses and schedule were administered for each format.

Materials and Methods

In this 5-week dose determination study, FUDR was delivered either as an intraperitoneal injection of free drug or as a subcutaneous injection of ATRIGEL®-FUDR in SCID mice. Mice either received 5 daily intraperitoneal (free drug) or 5 daily subcutaneous injections (polymer formulation) in doses of 50, 75, 100, and 150 mg/kg/day. Mice were monitored throughout the injection series and subsequent follow-up for toxicity symptoms. Each treatment group consisted of 5 mice.

Treatment Groups:

1. Control—vehicle only

Floxuridine (free drug in sterile saline (i.p., QD×5)]

2. 50 mg/kg

3. 75 mg/kg

4. 100 mg/kg

5. 150 mg/kg

Floxuridine (in polymer [s.c., QD×5)]

6. 50 mg/kg

7. 75 mg/kg

8. 100 mg/kg

9. 150 mg/kg

Total: 45 mice

Schedule:

All mice used for this study were screened for IgG production and “leaky mice were eliminated from the study group.
Mice were given i.p. or s.c. injection daily for 5 days with appropriate dose and formulation of Floxuridine.
Total injection volume for the intraperitoneal injection was 0.1/mL/mouse/injection.
Injection volume for subcutaneous injection was calculated assuming total release of drug from the polymer formulation.
Mice were monitored weekly for weight and toxicity symptoms (survival, general health, fur condition, etc.) for at least 8 weeks (7 weeks after the last injection).

Results and Discussion

In this dose determination study Floxuridine was delivered either as free drug or in a time-release format using the Atrix polymer sustained release system to SCID mice. The dose range consisted of 50 to 150 mg/kg/day administered daily for 5 days. In the previous dose determination study (Example 1) Floxuridine doses of 40 to 55 mg/kg/day×5 days did not result in toxicity. Mice received the drug by either intraperitoneal injection (free drug) or subcutaneous injection (drug/polymer formulation).

In the groups receiving free drug no toxicity symptoms have been observed. Only the group receiving 150 mg/kg/day (750 mg/kg/total) displayed weight lose (˜20 percent). All mice receiving the drug polymer formulation regardless of dose, died within 4 days of the completion of the injection series. When these mice were found by the animal care staff all had dried blood in both the oral and rectal areas. These mice also developed localized infection at the injection sites. Mouse weight, percent weight change, and dose data are shown below.

Atrix Laboratories, Inc. Floxuridine and Idoxuridine
in Atrigel ® Delivery System

[symbol]

| Dose | Dose | | | Mortality | Tumor Volume | Body Weight |
|---|---|---|---|---|---|---|
| (mg/kg) | Volume | | Route | (%) | Change | Change |
| 50 | 0.1 | mL | ip | 0 | NA | 0 |
| FUDR/ | | | | | | |
| Saline | | | | | | |
| q.d. × 5 | | | | | | |
| 75 | 0.1 | mL | ip | 0 | NA | 0 |
| FUDR/ | | | | | | |
| Saline | | | | | | |
| q.d. × 5 | | | | | | |
| 100 | 0.1 | mL | ip | 0 | NA | 0 |
| FUDR/ | | | | | | |
| Saline | | | | | | |
| q.d. × 5 | | | | | | |
| 150 | 0.1 | mL | ip | 0 | NA | −20% |
| FUDR/ | | | | | | |
| Saline | | | | | | |
| q.d. × 5 | | | | | | |
| 50 | 10 | μL | sc | 100 | NA | NA |
| FUDR/At | | | | D4 | | |
| q.d. × 5 | | | | | | |
| 75 | 15 | μL | sc | 100 | NA | NA |
| FUDR/At | | | | D4 | | |
| q.d. × 5 | | | | | | |
| 100 | 20 | μL | sc | 100 | NA | NA |
| FUDR/At | | | | D4 | | |
| q.d. × 5 | | | | | | |
| 150 | 30 | μL | sc | 100 | NA | NA |
| FUDR/At | | | | D4 | | |
| q.d. × 5 | | | | | | |

[image]

### Example 3

Floxuridine Delivered by the Atrix Polymer Sustained Release Delivery System to SCID Mice Bearing Subcutaneous SW480 (Human Colon Cancer) Tumors

Introduction

This Example was performed to determine whether Floxuridine delivered by intratumoral injection in a sustained release format via the Atrix polymer formulation will affect the growth of established tumors (subcutaneous SW480—Human Colon Cancer) in SCID mice.

Floxuridine, as a cell cycle dependent drug, is an ideal candidate for administration in a sustained release format. Floxuridine acts to interfere with the synthesis of DNA and to a lesser degree RNA. Since cells in a tumor are asynchronous, the ability to constantly supply Floxuridine to the tumor should markedly improve its effectiveness as an anti-tumor agent. In the clinic, Floxuridine is administered at 2 to 6 mg/kg given over 14 days. In order to approximate this in mice, 100 mg/kg given as a single intratumoral injection was used.

Materials and Methods

SCID mice were injected with 10×10⁶ SW480 (Human Colon Cancer) cells. When the average tumor diameter was approximately 0.5 cm, mice were divided into treatment groups such that the mean tumor volume in each group was equivalent and the drug was administered. Floxuridine/Polymer treatment consisted of a single intratumoral injection. The volume of Floxuridine/polymer used was calculated assuming total release of drug from the polymer and a drug concentration of 0.10 mg/μL of polymer. Equivalent free Floxuridine was administered as a single intraperitoneal injection. Each treatment group consisted of 8 SCID mice.

Treatment Groups:

1. Tumor bearing mice—Floxuridine/Polymer (100 mg/kg) i.t.—1×

2. Tumor bearing mice—Atrigel (equivalent to 100 mg/kg) i.t.—1×

3. Tumor bearing mice—Control—No injection

Total: 24 mice

Schedule:

All SCID mice used for this study were screened for IgG production and those mice producing IgG (“leaky”) were eliminated from the study group.
Mice were injected with 10×10⁶ SW480 cells.
When tumors achieved approximately 0.5 cm diameter, drug treatments were initiated. This was considered Treatment Day 1. All treatments consisted of a single injection.
Mice were monitored weekly for weight, tumor size and toxicity symptoms (survival, general health, fur condition, etc.) for the course of the study and the efficacy was compared.

Results and Discussion

This experiment was designed to measure the anti-tumor effect of Floxuridine delivered via the Atrix polymer sustained release system. All drug delivery was by intratumoral injection. Mice bearing established (˜0.5 cm diameter) subcutaneous colon (SW480) tumors were used for this study. Mice were randomized to equalize the tumor volume in each group prior to initiation of drug treatment. A single dose of 100 mg/kg, given as 1 injection, was used. A group of control mice received Atrigel without Floxuridine in a volume equivalent to the Floxuridine treatment group. Between Week 1 and 2 of drug treatment all mice receiving Floxuridine died. Control mice (tumor-bearing and those receiving Atrigel without Floxuridine) were all surviving. The experiment was terminated at this point.

At Week 1 tumor volume in control (untreated) mice had increased in volume to 369% of pre-treatment tumor volume. In Atrigel-treated mice increase was 267% and in mice treated with Floxuridine in Atrigel, the increase was only 228%. Floxuridine treated mice showed a weight loss of 21.5%, weight in the Atrigel alone group was unchanged (less than 1%) and control mice gained 3.6%.

The MTD in the literature for Floxuridine is 50 mg/kg/day×5 days for a cumulative dose of 250 mg/kg. This is 2.5 times the dose used in this Example. Data for dose volumes, mortality, tumor volume change, body weight change, mean tumor volumes and mean percent of pre-treatment tumor volumes are shown below.

Atrix Laboratories, Inc. Floxuridine and Idoxuridine
in Atrigel ® Delivery System
ATX [symbol] Mice

| Dose | Dose | | Mortality | Tumor Volume | Body Weight |
|---|---|---|---|---|---|
| (mg/kg) | Volume | Route | (%) | Change | Change |
| 100 | 20 μL | i.t. | 100 | −228% wk 1 | −22% |
| FUDR/ | | | wk 1-2 | | |
| At × 1 | | | | | |
| Atrigel × | 20 μL | i.t. | 0 | +267% wk 1 | 0 |
| 1 | | | | | |
| Control | NA | NA | 0 | +369% wk 1 | +3.6% |
| (no dose) | | | | | |

[image]

### Example 4

Floxuridine Delivered by the Atrix Polymer Sustained Release Delivery System to SCID Mice Bearing Subcutaneous SW480 (Human Colon Cancer) Tumors II

Introduction

This Example was designed to determine whether Floxuridine delivered by intratumoral injection in a sustained release format via the Atrix polymer formulation affects the growth of established tumors (subcutaneous SW480—Human Colon Cancer) in SCID mice.

Floxuridine, as a cell cycle dependent drug, is an ideal candidate for administration in a sustained release format. Floxuridine acts to interfere with the synthesis of DNA and to a lesser degree RNA. Because cells in a tumor are asynchronous, the ability to constantly supply Floxuridine to the tumor should markedly improve its effectiveness as an anti-tumor agent. In the clinic, Floxuridine is administered at 2 to 6 mg/kg given over 14 days. In order to approximate this in mice, 100 mg/kg given as a single intratumoral injection was used.

Materials and Methods

Mice were injected with 10×10⁶ SW480 (Human Colon Cancer) cells. When the average tumor diameter was approximately 0.5 cm, mice were divided into treatment groups such that the mean tumor volume in each group was equivalent and drug was administered. Floxuridine/Polymer treatment consisted of a single intratumoral injection. The volume of Floxuridine/polymer used was calculated assuming total release of drug from the polymer and a drug concentration of 0.10 mg/μL polymer. Equivalent free Floxuridine was administered as a single intraperitoneal injection. Each treatment group consisted of 8 SCID mice.

Treatment Groups:

1. Tumor bearing mice—Floxuridine/Polymer (100 mg/kg) i.t.—1×

2. Tumor bearing mice—Atrigel (equivalent to 100 mg/kg). i.t.—1×

3. Tumor bearing mice—Control—No injection

Total: 24 mice

Schedule:

All SCID mice used for this study were screened for IgG production and those mice producing IgG (“leaky”) were eliminated from the study group.
Mice were injected with 10×10⁶ SW480 cells.
When tumors achieved approximately 0.5 cm diameter, drug treatments were initiated. This was considered Treatment Day 1. All treatments consisted of a single injection.
Mice were monitored weekly for weight, tumor size and toxicity symptoms (survival, general health, fur condition, etc.) for at least 6 weeks and the efficacy was compared.

Results and Discussion

This experiment was designed to measure the anti-tumor effect of Floxuridine delivered via the Atrix polymer sustained release system. In the previous Example (Example 3) treatment with Floxuridine in Atrigel (100 mg/kg) resulted in 100% mortality. In this experiment mice were treated with 2 doses of Floxuridine in Atrigel (50 and 100 mg/kg). A group treated with a single injection of free Floxuridine at 100 mg/kg was also added. Drug delivery in Atrigel was by intratumoral injection. Free Floxuridine was administered by intraperitoneal injection. Mice bearing established (˜0.5 cm diameter) subcutaneous colon (SW480) tumors were used for this study. Mice were randomized to equalize the tumor volume in each group prior to initiation of drug treatment. A group of control mice received Atrigel without Floxuridine in a volume equivalent to the Floxuridine (100 mg/kg) treatment group. Between Week 1 and 2 of drug treatment all mice receiving Floxuridine in Atrigel died. Control mice (tumor-bearing and those receiving Atrigel without Floxuridine) and mice receiving free Floxuridine all survived. The experiment was terminated at the end of Week 2.

At Week 1 tumor volume in control (untreated) mice had increased in volume to 525% of pre-treatment tumor volume. In Atrigel-treated mice the increase was 240%. In mice treated with Floxuridine in Atrigel, increases in tumor volume of 387% for the 50 mg/kg treatments, and 206% for the 100 mg/kg treatments were observed. Tumors in mice treated with free Floxuridine increased to 244% of pre-treatment volume. Floxuridine treated mice showed weight losses of 16.3% (for 50 mg/kg treatments) and 17.8% (for 100 mg/kg treatments), the Atrigel alone group gained 2.7%. The weight of control mice was unchanged (less than 1%) and mice receiving free Floxuridine gained 3.4%.

The MTD in the literature for Floxuridine is 50 mg/kg/day×5 days for a cumulative dose of 250 mg/kg. This is 2.5 or 5 times the doses used in this study; 100 mg/kg of free Floxuridine had no negative effect. Data for dose volumes, mortality, tumor volume change, body weight change, mean tumor volumes and mean percent of pre-treatment tumor volumes are shown below.

ATX

[symbol]

| Dose | Dose | | Mortality | Tumor Volume | Body Weight |
|---|---|---|---|---|---|
| (mg/kg) | Volume | Route | (%) | Change | Change |
| 50 | 10 μL | i.t | 100 | −387% | −16% |
| FUDR/ | | | wk 1-2 | wk 1 | |
| At × 1 | | | | | |
| 100 | 20 μL | i.t. | 100 | −206% | −18% |
| FUDR/ | | | wk 1-2 | wk 1 | |
| At × 1 | | | | | |
| 100 | 20 μL | ip | 0 | +244% | +3% |
| FUDR/ | | | | wk 1 | |
| Saline × | | | | | |
| 1 | | | | | |
| 0 | 20 μL | i.t. | 0 | +240% | +3% |
| Atrigel × | | | | wk 1 | |
| 1 | | | | | |
| Control | NA | NA | 0 | +525% | 0 |
| (no dose) | | | | wk 1 | |

[image]

### Example 5

Floxuridine Delivered by the Atrix Polymer Sustained Release Delivery System to SCID Mice Bearing Subcutaneous PC-3 (Human Prostate Cancer) Tumors

Introduction

This Example was conducted to determine whether Floxuridine delivered by intratumoral injection in a sustained release format via the Atrix polymer formulation will affect the growth of established tumors (subcutaneous PC-3—Human Prostate Cancer) in SCID mice.

Floxuridine, as a cell cycle dependent drug, is an ideal candidate for administration in a sustained release format. Floxuridine acts to interfere with the synthesis of DNA and to a lesser degree RNA. Since cells in a tumor are asynchronous, the ability to constantly supply Floxuridine to the tumor should markedly improve its effectiveness as an anti-tumor agent. In the clinic, Floxuridine was administered at 2 to 6 mg/kg total dose given over 14 days. In order to approximate this in mice, 100 mg/kg given as a single intratumoral injection was used.

Materials and Methods

Mice were injected with 10×10⁶ PC-3 (Human Prostate Cancer) cells. When the average tumor diameter was approximately 0.5 cm, mice were divided into treatment groups such that the mean tumor volume in each group was equivalent and drug was administered. Floxuridine/Polymer treatment consisted of a single intratumoral injection. The volume of Floxuridine/polymer used was calculated assuming total release of drug from the polymer and a drug concentration of 0.10 mg/μL of polymer. Equivalent free Floxuridine was administered as a single intraperitoneal injection. Each treatment group consisted of 8 SCID mice

Treatment Groups:

1. Tumor bearing mice—Floxuridine/Polymer (100 mg/kg) i.t.—1×

2. Tumor bearing mice—Atrigel (equivalent to 100 mg/kg) i.t.—1×

3. Tumor bearing mice—Control—No injection

Total: 24 mice

Schedule:

All SCID mice used for this study were screened for IgG production and those mice producing IgG (“leaky”) were eliminated from the study group.
Mice were injected with 10×10⁶ PC-3 cells.
When tumors achieved approximately 0.5 cm diameter, drug treatments were initiated. This was considered Treatment Day 1. All treatments consisted of a single injection.
Mice were monitored weekly for weight, tumor size and toxicity symptoms (survival, general health, fur condition, etc.) for at least 6 weeks and the efficacy was compared.

Results and Discussion

This experiment was designed to measure the anti-tumor effect of Floxuridine delivered via the Atrix polymer sustained release system. All drug delivery was by intratumoral injection. Mice bearing established (˜0.5 cm diameter) subcutaneous prostate (PC-3) tumors were used for this study. Mice were randomized to equalize the tumor volume in each group prior to initiation of drug treatment. A single dose of 100 mg/kg, given as 1 injection, was used. A group of control mice received Atrigel without Floxuridine in a volume equivalent to the Floxuridine treatment group. Between Week 1 and 2 of drug treatment all mice receiving Floxuridine died. Control mice (tumor-bearing and those receiving Atrigel without Floxuridine) were all surviving. The experiment was terminated at this point.

At Week 1 tumor volume in control (untreated) mice had increased in volume to 567% of pre-treatment tumor volume. In Atrigel-treated mice the increase was 638% and in mice treated with Floxuridine in Atrigel increased by only 203%. Floxuridine treated mice showed a weight loss of 2.06%. Weight loss in the Atrigel alone group was 3.36% and in control mice, weight loss was 5.60%.

The MTD in the literature for Floxuridine is 50 mg/kg/da×5 days for a cumulative dose of 250 mg/kg. This is 2.5 times the dose used in this study. Data for dose volumes, mortality, tumor volume change, body weight change, mean tumor volumes and mean percent of pre-treatment tumor volumes are shown below.

Atrix Laboratories, Inc. Floxuridine and Idoxuridine
in Atrlgel ® Delivery System
ATX 9800 [symbol] Mice

| Dose | Dose | | Mortality | Tumor Volume | Body Weight |
|---|---|---|---|---|---|
| (mg/kg) | Volume | Route | (%) | Change | Change |
| 100 | 20 μL | i.t. | 100 | +203% | −2% |
| FUDR/ | | | wk 1-2 | wk 1 | |
| At × 1 | | | | | |
| 0 | 20 μL | i.t | 0 | +638% | −3% |
| Atrigel × | | | | wk 1 | |
| 1 | | | | | |
| Control | NA | NA | 0 | +567% | −6% |
| (no dose) | | | | wk 1 | |

[image]

### Example 6

Floxuridine Delivered by the Atrix Polymer Sustained Release Delivery System to SCID Mice Bearing Subcutaneous Hey (Human Ovarian Cancer) Tumors

Introduction

The Example was conducted to determine whether Floxuridine delivered by intratumoral injection in a sustained release format via the Atrix polymer formulation will affect the growth of established tumors (subcutaneous Hey—Human Ovarian Cancer) in SCID mice.

Floxuridine, as a cell cycle dependent drug, is an ideal candidate for administration in a sustained release format. Floxuridine acts to interfere with the synthesis of DNA and to a lesser degree RNA. Because cells in a tumor are asynchronous, the ability to constantly supply Floxuridine to the tumor should markedly improve its effectiveness as an anti-tumor agent. In the clinic, Floxuridine is administered at 2 to 6 mg/kg total dose given over 14 days. In order to approximate this in mice, 100 mg/kg given as a single intratumoral injection was used. The release profile for this formulation indicates that approximately 98% of the Floxuridine is released in 2 weeks, therefore a second drug dose was administered on Day 14.

Materials and Methods

In this study, SCID mice bearing established (˜0.5 cm diameter) SC ovarian (Hey) tumors were used in this study and were randomized to equalize the tumor volume prior to initiation of drug therapy. Doses of 50 and 100 mg/kg were administered intratumorally. Floxuridine in saline at 100 mg/kg was administered by intraperitoneal injection. ATRIGEL® alone was administered in a volume equal to the 100 mg/kg group.

Mice were injected with 10×10⁶ Hey (Human Ovarian Cancer) cells. When the average tumor diameter was approximately 0.5 cm, mice were divided into treatment groups such that the mean tumor volume in each group was equivalent and drug was administered. Floxuridine/Polymer treatment consisted of a two intratumoral injections (100 mg/kg each) given on days 1 and 14. The volume of Floxuridine/polymer used was calculated assuming total release of drug from the polymer and a drug concentration of 0.10 mg/μL of polymer. Equivalent free Floxuridine was administered as two intraperiteneal injections (100 mg/kg each), also given on Days 1 and 14. Each treatment group consisted of 8 SCID mice.

Treatment Groups:

1. Tumor bearing mice—Floxuridine/Polymer (100 mg/kg) i.t.—2×

2. Tumor bearing mice—Floxuridine (100 mg/kg) i.p.—2×

3. Tumor bearing mice—Control—No injection

Total: 24 mice

Schedule:

All SCID mice used for this study were screened for IgG production and those mice producing IgG (“leaky”) were eliminated from the study group.
Mice were injected with 10×10⁶ Hey cells.
When tumors achieved approximately 0.5 cm diameter, drug treatments were initiated. This was considered Treatment Day 1. All treatments consisted of two injections (Days 1 and 14).
Mice were monitored weekly for weight, tumor size and toxicity symptoms (survival, general health, fur condition, etc.) for at least 5 weeks and the efficacy was compared

Results and Discussion: This experiment was designed to measure the anti-tumor effect of Floxuridine delivered via the Atrix polymer sustained release system. In a previous experiment (Example 3) treatment with Floxuridine in Atrigel (10 mg/kg) resulted in 100% mortality. In this experiment mice were treated with 2 doses of Floxuridine in Atrigel (50 and 100 mg/kg). A group treated with single injection of free Floxuridine at 100 mg/kg was also added. Drug delivery in Atrigel was by intratumoral injection. Free Floxuridine was administered by intraperitoneal injection. Mice bearing established (˜0.5 cm diameter) subcutaneous breast cancer (Hey) tumors were used for this study. Mice were randomized to equalize the tumor volume in each group prior to initiation of drug treatment. A group of control mice received Atrigel without Floxuridine in a volume equivalent to the Floxuridine (100 mg/kg) treatment group. Between Week 1 and 2 of drug treatment 80% of mice receiving Floxuridine (50 mg/kg) in Atrigel, and 60% of mice receiving Floxuridine (100 mg/kg) in Atrigel died. Control mice tumor-bearing and those receiving Atrigel without Floxuridine) and mice receiving free Floxuridine were all surviving. The experiment was terminated at Week 4. Observations were continued in the surviving mice for an additional week.

At Week 1 tumor volume in control (untreated) mice had increased in volume to 176% of pre-treatment tumor volume. In Atrigel-treated mice the increase was 240%. In mice treated with Floxuridine in Atrigel, increases of 387% (for 50 mg/kg treatments) and were, and 206% (for 100 mg/kg treatments) were observed. Tumors in mice treated with free Floxuridine increased to 244% of pre-treatment volume. Floxuridine treated mice showed a weight losses of 12.33% (50 mg/kg) and 9.61% (100 mg/kg). Weights of control, Atrigel alone and free Floxuridine mice were unchanged (less than 1%).

At the termination of this experiment (Week 3), tumor volume increase control in mice was 445%, 354% in Atrigel alone and 377% in mice treated with free Floxuridine (100 mg/kg). The one remaining mouse treated with Floxuridine (50 mg/kg) in Atrigel displayed a volume increase of 177% and the 2 remaining mice in the Floxuridine (100 mg/kg) in Atrigel group averaged 148% increase.

The MTD in the literature for Floxuridine is 50 mg/kg/day×5 days for a cumulative dose of 250 mg/kg. This is 2.5 or 5 times the doses used in this study. No negative effect was found for treatment with 100 mg/kg of free Floxuridine. Data for dose volumes, mortality, tumor volume change, body weight change, mean tumor volumes and mean percent of pre-treatment tumor volumes are shown below.

ATX

[symbol]

| Dose | Dose | | Mortality | Tumor Volume | Body Weight |
|---|---|---|---|---|---|
| (mg/kg) | Volume | Route | (%) | Change | Change |
| 50 | 10 μL | i.t. | 80 | +387% | −12% |
| FUDR/ | | | wk 1-2 | wk 1 | |
| At × 2 | | | | | |
| 1 & 14 | | | | | |
| days | | | | | |
| 100 | 20 μL | i.t | 60 | +206% | −10% |
| FUDR/ | | | wk 1-2 | wk 1 | |
| At × 2 | | | | | |
| 1 & 14 | | | | | |
| days | | | | | |
| 100 | 20 μL | i.p. | 0 | +244% | 0 |
| FUDR/ | | | | wk 1 | |
| Saline × | | | | +377% | |
| 2 | | | | wk 3 | |
| 1 & 14 | | | | | |
| days | | | | | |
| 0 | 20 μL | i.t | 0 | +240% | 0 |
| Atrigel × | | | | wk 1 | |
| 1 | | | | +354% | |
| 1 & 14 | | | | wk 3 | |
| days | | | | | |
| Control | NA | NA | 0 | +176% | 0 |
| (no dose) | | | | wk 1 | |
| | | | | +455% | |
| | | | | wk 3 | |

[image]

### Example 7

Evaluation of the 7-Day Macroscopic Toxicity of Formulations Containing Floxuridine Delivered by a Single Subcutaneous Injection in C3H Male Mice

Introduction

This experiment was designed to determine the maximum tolerated dose (MTD) of FUDR in ATRIGEL® in C3H mice.

Materials and Methods

In a 7-Day study, three formulations were tested in 45 animals with three animals per treatment group. Each of the three formulations was tested at five different dose volumes. On Day 0 all mice were anesthetized, their dorsal thoracic (DT) area shaved, and injection sites wiped with isopropanol. Each animal received one 5, 10, 20, 25, 50, or 75 μL SC injection of the test article (TA) 1, TA 1.1, TA 2, or Control Article 1 formulation in the dorsal thoracic (DT) region. On Days 0, 1, 3, and 7 all mice were weighed and injection sites evaluated for any abnormalities including: redness, bleeding, swelling, discharge, bruising, and TA extrusion. On Days 0-7, mice were observed twice daily for signs of overt toxicity. Maximum tolerated dose (MTD) was defined as a 10% body weight loss and with clinical signs of overt toxicity.

Test Articles:

1) 38% 50/50 PLG (InV 0.26)/2% PEG5000-50/50 PLG (InV 0.79)/50% NMP with 10% (w/w) FUDR at dose volumes of 5, 10, and 25 μL for Groups I, II, and III;

1.1) 42.2% 50/50 PLG (InV 0.26)/2.2% PEG5000-50/50 PLG (InV 0.79)/55.6% NMP with 0.5% (w/w) FUDR at dose volumes of 10 and 20 μL for Groups IV and V;

2) 10% (w/v) FUDR in saline at dose volumes of 5, 10, 25, 50, and 75 μL;

3) 42.2% 50/50 PLG (InV 0.26)/2.2% PEG5000-50/50 PLG (InV 0.79)/55.6% NMP at dose volumes of 5, 10, 25, 50, and 75 μL.

Results and Discussion

The results of this study indicate that the maximum tolerated SC dose of ATRlGEL®-FUDR in C3H mice was exceeded in Groups I-V. The doses used were in the expected efficacious range for chemotherapeutic activity, therefore the C3H strain of ice may not be a suitable strain/species for further efficacy studies. Each of the three ATRIGEL®-FUDR formulations were tested at five different doses: 25, 55, 125, 2.5, and 5 mg/kg for Groups I-V, respectively. All mice in Groups I-V were sacrificed due to moribund condition or were found dead on Day 5, with overt toxicity signs of decreased activity, morbidity and coma. Body weight losses at Day 5 for these mice ranged from 11.2% to 21% in Groups I-V. Mice in Groups VI-X were dosed with FUDR in saline at doses of 24, 50, 120, 240, and 340 mg/kg, respectively. Mice in Groups XI-XV were dosed with ATRIGEL® alone at dose volumes of 5, 10, 25, 50 and 75 μL. Overt toxicity observations for mice in Groups VI-XV were unremarkable for the duration of the study. Test site observations were unremarkable for all groups.

### Example 8

Determination of the 28-Day Release Kinetics of an ATRIGEL® Formulation Containing Floxuridine Following SC Injection in Rats.

Introduction

This experiment was designed to determine the 28-Day release kinetics of an ATRIGEL®-FUDR formulation in Spague Dawley rats. Additionally, the in vivo molecular weight change over 28 days of this formulation was determined by Gel Permeation Chromatography (GPC) analysis.

Materials and Methods

In this 28-day study, one test article (TA; 95% 50/50 PLG (IV 0.26)+5% PEG5000-70/30 PLG (IV 0.79) in NMP (45/55) w/10% FUDR) was tested in one group of 30 rats. Each animal was given a single 0.05 cc subcutaneous injection of the TA in the DT region with a 23-gauge needle. On each of Days 1, 3, 7, 14, 21, and 28, five animals were anesthetized with isoflurane and bled by cardiac puncture. Animals were then terminated with CO₂ and implants were recovered. The retrieved implants were analyzed for Molecular Weight changes by Gel Permeation Chromatography. Macroscopic subcutaneous tissue irritation was evaluated by gross examination of the implants and the surrounding tissues. The animals were also observed daily for any overt toxicity.

Manufacturer Information. Floxuridine (FUDR): Spectrum Quality Products; Lot MW0189; Poly (DL-lactide-co-glycolide), 50/50 PLG (InV 0.26): Birmingham Polymers, Lot 115-69-1; Poly (DL-lactide-co-glycolide), 70/30 PLG/PEG (InV 0.79): Birmingham Polymers, Lot D97132; NMP: International Specialty Products; Trace #06097B.

Results and Discussion

No animals were found dead or moribund during the course of the study. Weight loss occurred after TA administration and continued until Day 14. The animals recovered their original body weight by Day 21 and gained weight thereafter. The weight loss correlates very well with the systemic Floxuridine release profile. A fairly uniform amount of formulation was administered to each rat. The mean amount injected for all the rats was 53.5 mg. The formulation was quite easy to inject through a 23-gauge needle. Minimal vasodilation, erythema, and capsule formation were observed during the whole study period. Edema was the only tissue reaction that was greatly in evidence. Edema increased from minimal at Day 1 to mild at Day 3 for some rats. It further progressed to marked edema at Day 7 and then slowly resolved itself around Day 21. The cause of this edema is most likely due to the release of Floxuridine from the implant because it was highly correlated to the drug release profile of the TA. The formulation had a 31%. Continuous drug release was followed at an almost zero-order fashion up to Day 14 when only about 6% drug was still entrapped in the polymer implant. The remaining Floxuridine released at a very slow rate over the next two weeks and by the end of the study, only about 1% remained in the implant. Floxuridine in rat plasma was generally undetectable with the current RP-HPLC method due to the high detection limit (20 ng/mL) of the method combined with the short biological half life of the drug. Still, the pharmacological effect of Floxuridine was evident with the loss of body weight and the mild to marked edema to the local tissue during the first two weeks after drug administration. Thus, the current formulation not only had relatively low initial burst but also was able to control the subsequent Floxuridine release up to two weeks in an almost linear manner. The formulation is therefore very promising for intratumoral injection to achieve sustained local action against tumor cells. Because the formulation contains two polymers, two peaks were observed on the GPC chromatograms: one was the high MW PEG-PLG and the other PLG. The high MW peak started to disappear as early as Day 3. At Day 14, it became completely absent. In contrast, the MW of PLG decreased very slowly from 13,400 at Day 1 to 12,200 at the end the study. This agrees well with the implant microscopic observation that little change in the implant size was noticed. It will probably take 3-4 months for the polymer to be completely degraded.

The results showed that the formulation had a relatively low initial burst (about 31%) and a fairly constant rate of drug release for two weeks after administration. At the current dose level, Floxuridine plasma levels of the rats were generally lower than the RP-HPLC detection limit of 20 ng/mL for most of the samples analyzed. The formulation was found to be well tolerated in the rat model. These animals did experience temporal weight loss and mild to marked edema at the injection site in the first two weeks after administration, indicating the toxic effect of Floxuridine. In addition, GPC analysis showed that the PLG polymer degraded rather slowly while the PEG-PLG polymer disappeared quickly. Thus, the implant will remain in the body for quite a long time after all the Floxuridine has been released. A graph of the 28-day release profile of Floxuridine is shown below.

### Example 9

Evaluation of the 14-Day Release Kinetics of Three ATRIGEL® Formulations at Varied Drug Loading of 1%, 5% and 10% Floxuridine Delivered by a Single Subcutaneous Injection into Male Rats

Introduction

This experiment was designed to determine the 14-Day release kinetics of 3 ATRIGEL®-FUDR formulations in Spague Dawley rats.

Materials and Methods

In this 14-Day study, three formulations were tested in 60 animals with 20 animals per treatment group. Each rat received one 100 μL SC injection of appropriate TA in the DT region. On Days 0, 1, 3, 7 and 14 all rats were weighed and the injection sites of all rats evaluated. On Days 0-14, rats were observed twice daily for signs of overt toxicity. On Days 1, 3, 7 and 14, five rats per treatment group were euthanized with CO₂. Implants and test sites were characterized and documented. All animals were given a partial necropsy (abdominal cavity) and observations documented. Implants were analyzed for FUDR content and drug release profile determination.

Test Articles:

1) 42.2% 50/50 PLG (InV 0.26)/2.2% PEG5000-50/50 PLG (InV 0.79)/55.6% NMP with 1.0% (w/w) FUDR;

2) 45.0% 50/50 PLG (InV 0.26)/3.0% PEG5000-50/50 PLG (InV 0.79)/52.0% NMP with 5.0% (w/w) FUDR;

3) 42.2% 50/50 PLG (InV 0.26)/2.2% PEG5000-50/50 PLG (InV 0.79)/55.6% NMP with 10% (w/w) FUDR.

Results and Discussion

The results of this study indicate all three formulations had acceptable initial burst at Day 1, followed by continuous and almost complete drug release by Day 7. Drug loading apparently had some effect on drug release: it did not affect initial burst on Day 1 but subsequent release tended to be faster for the 1% drug loading formulation than the higher drug loading formulations. In addition, increase in polymer content in the formulation apparently decreased initial burst. However, the duration of release was not affected. It is noted that duration of release for all three formulations was only 7 days instead of the previously observed 14 days (Example 8). The use of the PEG5000-50/50 PLG(InV 0.79) in the current study instead of the PEG5000-70/30 PLG(InV 0.79) may have caused that difference.

### Example 10

Evaluation of the 14-Day Macroscopic Toxicity of Formulations Containing Floxuridine Delivered by a Single Subcutaneous Injection in Fischer 344 Male Rats

Introduction

This experiment was designed to determine the maximum tolerated dose (MTD) of FUDR in ATRIGEL® in Fischer 344 Rats.

Materials and Methods

In this 14-Day study, one formulation was tested in eighteen (18) animals with three animals per treatment group. Each animal in Groups I-III and VI received one 12, 25, or 50 μL subcutaneous injection of the TA or CA formulation in the DT region. On Days 0, 1, 3, 5, 7, 10 and 14 rats in Groups I-III and VI were weighed and the injection sites evaluated for any abnormalities including: redness, bleeding, swelling, discharge, bruising, and TA extrusion. Rats were observed twice daily for signs of overt toxicity by Inhausen Research Institute (IRI) personnel for the duration of the study. The MTD determination of this study was achieved with Groups I-III and VI, therefore rats in Groups IV and V were used to determine the 7-Day release kinetics, thus providing a more accurate assessment of the 14-Day release kinetics. Rats in Group IV and V received approximately 65 μL of TA 1.

Test Articles:

1) 42.2% 50/50 PLG (InV 0.26) 2.2% PEG5000-50/50 PLG (InV 0.79)/55.6% NMP with 1.0% (w/w) FUDR;

2) 45.0% 50/50 PLG (InV 0.26) 3.0% PEG5000-50/50 PLG (InV 0.79)/52.0% NMP with 5.0% (w/w) FUDR;

3) 42.2% 50/50 PLG (InV 0.26) 2.2% PEG5000-50/50 PLG (InV 0.79)/55.6% NMP with 10% (w/w) FUDR.

Results and Discussion

Analysis of the retrieved implants shows the formulation releasing FUDR very quickly in the first three days followed by a slow and continuous release up to Day 14. The percent of drug released at Days 3, 7, and 14 were 73.4%, 94.8%, and 99.8% respectively. The rate of release demonstrated in the current study is apparently much faster than the release in a previous study, Example 8. Drug release at Day 7 for the Example 8 formulation was only 60% versus 95% for this study. It was noted that the formulation used in Example 8 had very high molecular weights of 187,200 and 14,000 for the two polymers. The formulation used in Example 8 was not sterilized. Although the same lots of polymers were used in the current study, molecular weight of the formulation may be decreased significantly due to five-years storage as well as sterilization by irradiation.

### Introduction to Examples 11-19

Examples 11-19 were conducted with ATRIGEL® Floxuridine (FUDR) in male Sprague Dawley rats. All doses were administered by subcutaneous injection in the dorsal lumbar or dorsal thoracic region. The main purpose of the studies was to evaluate the release kinetics of FUDR from varying ATRIGEL® formulations in-situ. Clinical observations, including survival, body weights and injection site reactions, were also evaluated. Doses (FUDR) administered were approximately 20 mg/kg administered in one 50 μL injection. In some cases, two 50 μL injections (40 mg/kg) were administered. This yielded approximately five mg per animal. The formulations were 10% FUDR (w/w) and delivered to rats of an average weight of 250 grams. The needle size required for injection of this formulations viscosity was 20 to 23-gauge (1″ needle). There were no mortalities. Body weight effects ranged from no effect to minimal decreases for the 24-hour studies and transient weight losses (through Day 14) of up to 15% in the 28-day studies. Injection site reactions included minimal to marked erythema, edema, vasodilation, and capsule formulation but no apparent necrosis or ulceration.

### Example 11

Injection Site Reaction and Release Kinetics (Implant Retrieval)

Materials and Methods

This Example was conducted using 50 Sprague Dawley rats (two injections per rat). The duration of the study was 24 hours. Twenty test articles were used.

Test Articles:

1) 50% 75/25 PLC (IV 0.33)/50% NMP

2) 50% 75/25 PLC (IV 0.33)/50% DMSO

3) 50% 75/25 PLC (IV 0.33)/50% DMA

4) 50% 50/50 PLG (IV 0.16)/50% TG

5) 40% 50/50 PLGH (IV 0.20)/60% NMP

6) 40% 50/50 PLGH (IV 0.20)/60% DMSO

7) 40% 50/50 PLGH (IV 0.20)/60% DMA

8) 40% 50/50 PLGH (IV 0.20)/60% TG

9) 40% 50/50 PLGH (IV 0.10)/60% NMP

10) 40% 50/50 PLGH (IV 0.10)/60% DMSO

11) 40% 50/50 PLGH (IV 0.10)/60% DMA

12) 30% 50/50 PLGH (IV 0.10)/70% TG

13) 50% 85/15 PLG (IV 0.09)/50% NMP

14) 50% 85/15 PLG (IV 0.09)/50% DMSO

15) 50% 85/15 PLG (IV 0.09)/50% DMA

16) 50% PLA-H (IV 0.20)/50% NMP

17) 50% PLA-H (IV 0.20)/50% DMSO

18) 50% PLA-H (IV 0.20)/50% DMA

19) 50% 50/50 PLG (IV 0.09)/50% NMP

20) 50% 50/50 PLG (IV 0.09)/50% DMSO

Results and Discussion

No animals were found dead or moribund during the course of the study. All groups showed a slight decrease in weight. The average amount injected for each group was between 12.8 mg and 61.7 mg. Group II-DL and Group IV DT-injections were much lower due to injection difficulty and/or limited formulation. There was marked capsule formation associated with every group except Group III-DL, Group IV-DL, and Group V-DL. Edema was fairly low for each group and vasodilation ranged from mild to moderate. Also, there was more marked erythema than any other tissue response. Only five TAs showed an initial burst lower than 80%. The best formulation was the 50% PLA-H/50% DMSO (Formulation 17), which yielded a burst of 50%.

### Example 12

Injection Site Reaction and Release Kinetics (Implant Retrieval) II

Materials and Methods

This Example was conducted using 40 Sprague Dawley rats. The duration of the study was 24 hours. Eight test articles were used.

Test Articles:

1) 50% 75/25 PLC (IV 0.33)/50% NMP

2) 50% 75/25 PLC (IV 0.33)/50% DMA

3) 50% PLA (MW 2000)/50% DMA

4) 50% PLA (MW 2000) 10% Myverol/40% DMA

5) 50% PLA-H (IV 0.20)/10% Myverol/40% DMA

6) 50% PLA-H (IV 0.20)/10% Ethyl Heptanoate/40% DMA

7) 50% PLA-H (IV 0.20)/50% DMSO

8) 50% PLA-H (IV 0.20)/50% DMA

Results and Discussion

No animals were found dead or moribund during the course of the study. All weights stayed within normal parameters. The target injection amount was 50 mg. The average amount injected per group was between 48.4 mg and 60.8 mg. One animal in Group III and four animals in Group IV had difficulty with injection. These difficulties arose because Formulations 3 and 4 were dispersions. A larger gauge needle was used to inject these formulations (20-gauge versus the 22-gauge used for all the other formulations\) and they were heated slightly before syringe filling, but even these measures were not enough to give a good delivery. Vasodilation, erythema, and edema were present in all groups, in severity ranging from minimal to marked (one instance of marked), with no apparent pattern. There was minimal capsule formation in Groups I, III, VI, and VIII and one instance of moderate capsule formation in Group III, The degree of tissue reaction at the 24-hour time point was not unexpected for injected polymer formulations. Groups I and II had initial bursts that were 5% higher than in ATRS-191 while Group VIII had a burst 3% lower than in Example 11. Group VII, the best formulation from Example 11—where it had an average release of 50.1%, had an average release of 83.2% in this study. Of the four new formulations tested (Groups III, IV, V, and VI), only Group VI had an average 24 hour release less than 80%.

### Example 13

Injection Site Reaction and Release Kinetics (Implant Retrieval) III

Materials and Methods

This Example was conducted using 15 Sprague Dawley rats (2 injections per rat). The duration of the study was 24 hours. Six test articles were used.

Text Articles:

1) 50/50 PLG (IV 0.26)/NMP w/5% Pluronic F127

2) 50/50 PLG (IV 0.26)/NMP w/1% Lecithin

3) 95% 50/50 PLG (IV 0.26)+5% PEG-5000-50/50 PLG (IV 0.81)/NMP (40/60)

4) 90% 50/50 PLG (IV 0.26)+10% PEG-5000-50/50 PLG (IV 0.81)/NMP (40/60)

5) 95% 50/50 PLG (IV 0.26)+5% PEG-5000-50/50 PLG (IV 0.81)/NMP (40/60) w/1% Lecithin

6) 90% 50/50 PLG (IV 0.26)+10% PEG-5000-50/50 PLG (IV 0.81)/NMP (40/60) w/1% Lecithin

Results and Discussion

No animals were found dead or moribund during the course of the study. All groups showed a minimal decrease in weight. The average amount injected for each group varied between 50.3 and 56.8 mg. There were no injection difficulties encountered in the experiment. Minimal to mild capsule formation was present in every group with no particular pattern being observed. Almost all tissue reactions were of minimal to mild nature. The incident of vasodilation, erythema, and edema occurred at about same rate and generally, only one or two categories of such tissue reactions happened to a single test site. It appears that the addition of 1% lecithin does not alter the 24-hour release kinetics in any significant way. However, PEG-PLG drastically reduces drug burst from ˜85% to ˜45%. Compared to an in vitro study in eggs, the same drug release trend was apparent, although the amount released is almost twice as high in vivo. Incorporating 5% or 10% PEG-PLG (iv 0.81) in the ATRIGEL® formulation can significantly reduce the initial burst of Floxuridine. However, such effect was not observed with either Pluronic® F 127 or Lecithin.

### Example 14

Injection Site Reaction and Release Kinetics (Implant Retrieval) IV

Materials and Methods

This Example was conducted using 15 Sprague Dawley rats (2 injections per rat). The duration of the study was 24 hours. Six test articles were used.

Test Articles:

1) 50/50 PLG (IV 0.16)/NMP w/2% PEG8-Stearate

2) 95% 50/50 PLG (IV 0.16)+5% PEG-5000-50/50 PLG (IV 0.81)/NMP

3) 90% 50/50 PLG (IV 0.16)+10% PEG-5000-50/50 PLG (IV 0.81)/NMP

4) 95% 50/50 PLG (IV 0.12)+5% PEG-5000-50/50 PLG (IV 0.81)/NMP

5) 90% 50/50 PLG (IV 0.12)+10% PEG-5000-50/50 PLG (IV 0.81)/NMP

6) 90% 50/50 PLG-H (IV 0.20)+10% PEG-5000-50/50 PLG (IV 0.81)/NMP

Results and Discussion

No animals were found dead or moribund during the course of the study.

There were no unusual weight changes over the 24-hour period of this study. The average amount injected for each group was between 50.6 and 55.4 mg. There were no injection difficulties encountered. Minimal or no or capsule formation was observed for all the groups. Almost all tissue reactions were of minimal to mild nature. Vasodilation happened more frequently than erythema and edema occurred only occasionally. The addition of 2% peg 400-stearate does not appear to reduce drug burst. However, when PEG-PLG was added to the formulations made from the PLG IV 0.16 polymer, the burst was reduced to about 60%. This burst-reducing effect of PEG-PLG was also observed for the PLGH polymer. These results are consistent with the results of Example 13, where the effect of PEG-PLG was even more pronounced. However, it appears that PEG-PLG works only with polymers that have moderate or high molecular weight, not with very low molecular weight PLGs such as the IV 0.12 polymer in the current study.

The initial burst of floxuridine can be significantly reduced by adding 5% or 10% PEG-PLG (IV 0.81 ) to an ATRIGEL® formulation made up of moderate or high molecular weight (IV>0.16) PLGs or PLGHs. As a general trend, the low molecular weight PLGs yielded a higher burst than the moderate or high molecular weight PLGs. Very low molecular weight PLGs always produced more than a 90% initial burst, even with the addition of PEG-PLG. No formulation in the study showed acceptable release kinetics.

### Example 15

Injection Site Reaction and Release Kinetics (Implant Retrieval) V

Materials and Methods

This Example was conducted using 20 Sprague Dawley rats (2 injections per rat). The duration of the study was 24 hours. Eight test articles were used.

Test Articles:

1) 95% D,L-PLAH (IV 0.20)+5% PEG-50/50 PLG (IV 0.81) in NMP (40/60)

2) 90% D,L-PLAH (IV 0.20)+10% PEG-50/50 PLG (IV 0.81) in NMP (40/60)

3) 65% 50/50 PLG (IV 0.26)+35% 50/50 PLG (IV 0.16)+5% PEG-5000-50/50 PLG (IV 0.81) in NMP (40/60)

4) 47.5% 50/50 PLG (IV 0.26)+47.5% 50/50 PLG (IV 0.16)+5% PEG-5000-50/50 PLG (IV 0.81) in NMP (40/60)

5) 30% 50/50 PLG (IV 0.26)+65% 50/50 PLG (IV 0.16)+5% PEG-5000-50/50 PLG (IV 0.81) in NMP (40/60)

6) 65% 50/50 PLG (IV 0.26)+30% 50/50 PLG (IV 0.16)+5% PEG-5000-50/50 PLG (IV 0.81) in NMP (40/60)

7) 47.5% 50/50 PLG (IV 0.26)+47.5% 50/50 PLG (IV 0.20)+5% PEG-5000-50/50 PLG (IV 0.81) in NMP (40/60)

8) 30% 50/50 PLG (IV 0.26)+65% 50/50 PLGH (IV 0.20)+5% PEG-5000-50/50 PLG (IV 0.81) in NMP (40/60)

Results and Discussion

No animals were found dead or moribund during the course of the study. There were no unusual weight changes over the 24-hour period of this study. The average amount injected for each group was between 50.4 and 54.4 mg. There were no injection difficulties encountered in the experiment. Minimal or no capsule formation was observed for all the groups. Most tissue reactions were of minimal to mild nature, but occasionally moderate, or even marked, vasodilation or erythema was notice. There did not appear to be a pattern to the tissue reaction. All the PLAH-based formulations had very high initial bursts, making PLAH inappropriate for use in prolonging Floxuridine release. For the formulations made of polymer mixtures, the higher the content of the 0.16 PLG, the higher the burst. However, it should be noted that the blend with 30% 0.16 PLG in this study had a comparable burst to the formulation used in Example 13 that was made entirely of IV 0.26 PLG. Therefore, it is possible to modify the release kinetics as well as formulation viscosity without increasing initial burst. However, this modification was probably polymer-specific, because, when PLGH was blended with the 0.26 PLG polymer, its effect on burst was obvious even at the low PLGH concentration level of 30%.

The delivery of Floxuridine from ATRIGEL® formulations, with the benefits of prolonged release and a lower viscosity, may be made possible by a mixture of two polymers plus 5% PEG-PLG. When 30% 0.16 PLG was mixed with 65% 0.26 PLG plus 5% PEG-PLG, the formulation had a lowered burst, similar to the one made by 95% 0.26 PLG plus 5% PEG-PLG in a previous study, while having decreased viscosity and possibly faster degradation. The addition of PLGH can increase drug burst significantly even at a low concentration level. PLAH was not well suited for Floxuridine formulations due to its high burst.

### Example 16

Injection Site Reaction and Release Kinetics (Implant Retrieval) VI

Materials and Methods

This Example was conducted using 30 Sprague Dawley rats. The duration of the study was 28 days. One test article was used (65% 50/50 PLG (IV 0.26)+35% 50/50 PLG (IV 0.16)+5% PEG-5000-50/50 PLG (IV 0.81) in NMP (40/60) w/10% FUDR).

Results and Discussion

No animals were found dead or moribund during the course of this study. Weight loss occurred immediately after TA administration. All animals continuously lost body weight until Day 7 and maintained that lower body weight up to Day 14. The animals slowly recovered their original body weight by Days 21 and 28. The weight loss correlates very well with the Floxuridine release profile. The planned injection amount was 50 mg (about 0.05 cc). The mean amount injected for all the rats was 51.1 mg. The formulation was quite easy to inject through a 23-gauge needle. Only minimal to mild tissue reactions were observed and these reactions were mostly resolved after Day 21. Occasional capsule formation was noticed in some rats at later time points, but they were also minimal to mild in nature. The formulation had a 51% initial burst, which agreed with the previous experimental value. Continuous drug release followed at a fairly constant rate up to Day 7, when about 13% drug still remained in the implant. The remaining Floxuridine released at a very slow rate over the next three weeks. Surprisingly, the plasma assay revealed no detectable Floxuridine level at any time point. This is probably due to the high detection limit (20 ng/mL) of the current RP-HPLC method combined with the fact that Floxuridine has a very short biological half life. Still, the pharmacological effect of Floxuridine was evident with the loss of body weight during the first three weeks after drug administration. Thus, the current formulation can prolong Floxuridine release but it still releases too fast, especially in the initial 24 hours after administration.

The initial burst of the formulation (roughly 51%) and the rate of release thereafter were high and therefore most of the drug (87%) was released by one week after administration. In addition, at the current dose level, the plasma levels of Floxuridine were found to be lower than the RP-HPLC detection limit of 20 ng/mL for all the samples analyzed. The formulation was well tolerated in the rat model since no major macroscopic tissue reactions were observed. The animals did experience temporary weight loss in the first three weeks after administration, indicating the toxic effect of Floxuridine.

### Example 17

Injection Site Reaction and Release Kinetics (Implant Retrieval) VII

Materials and Methods

This Example was conducted using 15 Sprague Dawley rats (2 injections per rat). The duration of the study was 24 hours. Six test articles were used.

Test Articles:

1) 50/50 PLG (IV 0.35) in NMP (40/60)

2) 95% 50/50 PLG (IV 0.35)+5% PEG-5000-50/50 PLG (IV 0.41) in NMP (40/60)

3) 95% 50/50 PLG (IV 0.35)+5% PEG-5000-50/50 PLG (IV 0.81) in NMP (40/60)

4) 95% 50/50 PLG (IV 0.35)+5% 50/50 PLG (IV 0.61) in NMP (40/60)

5) 95% 50/50 PLG (IV 0.35)+5% 50/50 PLG (IV 0.70) in NMP (40/60)

6) 95% 50/50 PLG (IV 0.35)+5% 50/50 PLG (IV 1.03) in NMP (40/60)

Results and Discussion

No animals were found dead or moribund during the course of the study. One group of rats showed some weight gain, all other groups exhibited a minimal decrease in weight. The average amount injected for each group was between 48.6 and 58.1 mg. The variations were caused by the crude volume marks on the syringe rather than injection difficulties. Also, an air bubble in the needle may have caused a marked low amount as in the case of the first rat in Group I. The amount injected within each group was fairly consistent and there were no injection difficulties encountered in the experiment. No capsule formation was observed for all the groups. Most tissue reaction were of minimal to mild nature, but occasionally moderate or even marked erythema was noticed. Erythema occurred in more than 70% of the sites. Vasodilation occurred in around 26% of the sites, and edema only occurred in about 13% of the sites. It appears that burst was reduced only with addition of the high molecular weight PEG-PLG. The low molecular weight IV 0.4 PEG-PLG did not reduce but in fact promoted drug burst just as predicted in vitro. However, it is surprising to see that addition of high molecular weight PLGs had no effect on Floxuridine burst at all, no matter what molecular weight of PLGs added. This was in direct contrast to the results from in vitro, where the high molecular weight of PLGs produced release profiles comparable to IV 0.81 PEG-PLG in phosphate buffers with 40% methanol. Therefore no correlation may be established between the in vitro and in vivo studies. It is also interesting to note that although TA #3 employed a more viscous IV 0.35 PLG, its burst (44.4%) was almost same as the one using a less viscous IV 0.26 PLG (45.3% burst, TA #3 of Example 13). Since degradation is already too long for the IV 0.26 PLG, the formulation in the current study may not have any advantages over the previous formulations.

Addition of high molecular weight PLGs as additives cannot reduce floxuridine burst of ATRIGEL formulations. Low burst can only be achieved with the addition of high molecular weight PEG-PLG such as PEG5000-50/50 PLG (iv 0.81), but not with low molecular weight ones. The use of PLG (iv 0.35) in the ATRIGEL formulation yielded no improved burst as compared to the one based on PLG (iv 0.26). Therefore, the formulations in this Example are generally similar to previous Examples.

### Example 18

Injection Site Reaction and Release Kinetics (Implant Retrieval) VIII

Materials and Methods

This Example was conducted using 15 Sprague Dawley rats (2 injections per rat). The duration of the study was 24 hours. Six test articles were used.

Test Articles:

1) 50/50 PLG (IV 0.26) in NMP (50/50)

2) 98% 50/50 PLG (IV 0.26)+2% PEG-5000-50/50 PLG (IV 0.81) in NMP (50/50)

3) 95% 50/50 PLG (IV 0.26)+5% PEG-5000-50/50 PLG (IV 0.81) in NMP (50/50)

4) 90% 50/50 PLG (IV 0.26)+10% PEG-5000-50/50 PLG (IV 0.81) in NMP (50/50)

5) 95% 50/50 PLG (IV 0.26)+5% PEG-5000-50/50 PLG (IV 0.79) in NMP (50/50)

6) 95% 50/50 PLG (IV 0.26)+5% PEG-5000-50/50 PLG (IV 0.81) in Propylene Carbonate (40/60)

Results and Discussion

No animals were found dead or moribund during the course of the study. All groups showed a minimal decrease in weight. The average amount injected for each group was between 45.7 and 59.4 mg. Due to high polymer content, these formulations were quite viscous and thus were difficult to push out the syringe. Hidden air bubbles in the needle may have caused the two occasions of unusually low injection amount. There was little incidence of capsule formation with only one mild capsule seen in TA 5. Most groups only experienced minimal to mild tissue reactions in all three categories of vasodilation, erythema, and edema except the TA 6 group. Each animal in TA 6 (the propylene carbonate group) showed some erythema with two animals showing marked erythema reactions. When propylene carbonate was used in the formulation, a very high burst was obtained. All the NMP based formulations yielded smaller bursts; increasing the amount of PEG-PLG to 5% resulted in even smaller bursts. However, at 10% the burst increased. It is thus concluded that 5% PEG-PLG is the optimum amount to lower the burst of Floxuridine. In addition, when another PEG-PLG with similar intrinsic viscosity was used, approximately the same burst was attained. So the molecular weight of PEG-PLG is more important than the polymer composition in reducing the drug burst. In Example 13, the formulation of PLG (iv 0.26) with 5% PEG-PLG in 60% NMP (w/w) had a burst of ˜45%. With these same polymers in 50% NMP, the present study showed a much lower burst of ˜29%. Such a formulation is more viscous and may be difficult to inject with a higher polymer concentration. A balance must be achieved between low drug burst and injectability.

The results indicated that addition of high MW PEG-PLG as an additive dramatically reduced the Floxuridine initial burst from ATRIGEL® formulations. The maximum burst-reducing effect of PEG-PLG was achieved at only 5% of the total polymer amount in the formulation. The PEG-5000-70/30 PLG (IV 0.79) was found to be as effective as the PEG-5000-50/50 PLG (IV 0.81) in reducing drug burst. Burst was also reduced with an elevated polymer/NMP ratio. For example, at a 50/50 (w/w) polymer/NMP ratio, a Floxuridine formulation with PLG (IV 0.26) and 5% PEG/PLG had a burst of ˜29%, whereas a low ratio (40/60) formulation had a burst ˜45%. In addition, propylene carbonate was found to be unsuitable as a solvent for Floxuridine ATRIGEL® formulations.

### Example 19

Injection Site Reaction and Release Kinetics (Implant Retrieval) IX

Materials and Methods

This Example was conducted using 15 Sprague Dawley rats (2 injections per rat). The duration of the study was 24 hours. Six test articles were used.

Test Articles:

1) 95% 50/50 PLG (IV 0.26)+5% PEG5000-50/50 PLG (IV 0.81) in NMP (45/55) w/10% FUDR

2) 95% 50/50 PLG (IV 0.26)+5% PEG5000-70/30 PLG (IV 0.79) in NMP (45/55) w/10% FUDR

3) 95% 50/50 PLGH (IV 0.20)+5% PEG5000-70/30 PLG (IV 0.79) in NMP (45/55) w/10% FUDR

4) 95% 50/50 PLGH (IV 0.20)+5% PEG5000-70/30 PLG (IV 0.79) in NMP (50/50) w/10% FUDR

5) 95% 50/50 PLGH (IV 0.30)+5% PEG5000-70/30 PLG (IV 0.79) in NMP (35/65) w/10% FUDR

6) 95% 50/50 PLGH (IV 0.30)+5% PEG5000-70/30 PLG (IV 0.79) in NMP (40/60) w/10% FUDR

Results and Discussion

No animals were found dead or moribund during the course of the study. All groups showed a minimal decrease in weight. The average amount injected for each group varied between 52.2 and 55.4 mg and the standard deviation was 4.0 mg or less. All formulations were quite easy to inject through a 23-gauge needle. There was little capsule formation for all the TAs tested. Most groups experienced only minimal to mild reactions in categories of vasodilation, erythema, and edema. No major tissue reactions were found in any animals. Both TAs made of PLG (IV 0.26) produced an acceptable low burst of 27%. These results were comparable to formulations of identical composition (but higher total polymer content) run in Example 18, where a 50% total polymer concentration was used instead of the current 45%. Therefore, it is possible to reduce polymer concentration to improve syringeability without altering the release profile much. In addition, here again it is shown that TA 1 and TA 2 have almost identical burst although different PEG-PLGs were used in their formulations. This confirms our earlier findings that these two PEG-PLGs are similar in their burst-reducing ability. Such ability thus appears more related to the MW of PEG-PLG than its molecular structure. All TAs made of PLGH polymers showed larger initial bursts than the PLG formulations. They contained the same 5% PEG-PLG (IV 0.79) as TA 2. the PLGH polymers used in these formulations also had a similar MW as the PLG (IV 0.26) polymer. The difference in burst is probably due to the carboxyl end group that increases the hydrophilicity of the polymer. The use of higher MW PLGH will not result in a smaller burst because current data clearly show that the higher the MW of PLGH, the larger the burst. The PLGH polymer has the advantage of fast degradation in vivo.

The results showed that it is possible to reduce the polymer concentration while leaving the initial burst unaffected and an increase in polymer concentration might not translate into decreased initial burst. The effect of polymer concentration on initial burst may be polymer specific and concentration related. The study confirmed the previous finding that the initial burst-reducing effect was almost identical for PEG5000-70/30 PLG (IV 0.79) and PEG5000-50/50 PLG (IV 0.81). The MW of PEG-PLW is a better indicator of the burst-reducing ability than the molecular structure. For Floxuridine, the carboxyl end capped PLGH was not as good as the corresponding PLG in controlling the initial burst, therefore, PLGH is not suitable for Floxuridine ATRIGEL® formulations. Successful formulations can be prepared using PLG (IV 0.26) and either 0.79 or 0.81 IV PEG-PLG as an additive.

The Examples demonstrate that Floxuridine delivered as Atrigel®-FUDR results in a lower Maximum Tolerated Dose than FUDR delivered as a free drug. The lower dose of Floxuridine results in fewer side-effects from the treatment. The formulation was found to be well tolerated in the rat model. Additionally, Floxuridine delivered by Atrigel® to tumor bearing mice was able to decrease the rate of tumor growth by approximately 50%, as compared to tumor bearing mice treated with Floxuridine as a free drug, Atrigel® alone, and untreated controls. A formulation was developed with a low initial drug-release burst (˜31%) and a constant rate of drug release for two weeks after administration.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any sub-combination.

1. A flowable composition suitable for use as a controlled release implant, the composition comprising:
(a) a biodegradable, biocompatible thermoplastic polymer that is at least substantially insoluble in aqueous medium, water or body fluid;
(b) a cell-cycle dependent biological agent, a schedule-dependent biological agent, a metabolite thereof, a pharmaceutically acceptable salt thereof, or a prodrug thereof; and
(c) a biocompatible organic liquid, at standard temperature and pressure, in which the thermoplastic polymer is soluble.

2. The composition of claim 1 wherein the biodegradable, biocompatible thermoplastic polymer is a linear polymer.

3. The composition of claim 1 wherein the biodegradable, biocompatible thermoplastic polymer is a branched polymer.

4. The composition of claim 1 wherein the biodegradable, biocompatible thermoplastic polymer has a formula incorporating monomeric units selected from the group of lactides, glycolides, caprolactones, glycerides, anhydrides, amides, urethanes, esteramides, orthoesters, dioxanones, acetals, ketals, carbonates, phosphazenes, hydroxybutyrates, hydroxyvalerates, alkylene oxalates, alkylene succinates, amino acids, and any combination thereof; and the formula contains the monomeric units random or block order.

5. The composition of claim 1 wherein the biodegradable, biocompatible thermoplastic polymer is a polymer or copolymer of lactide monomeric units, caprolactone monomeric units, glycolide monomeric units, or any combination thereof.

6. The composition of claim 1 wherein the biodegradable, biocompatible thermoplastic polymer comprises a polymer selected from the group of polylactides, polyglycolides, polycaprolactones, polydioxanones, polycarbonates, polyhydroxybutyrates, polyalkyene oxalates, polyanhydrides, polyamides, polyesteramides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, poly(methyl vinyl ether), polyesters, polyalkylglycols, copolymers thereof, block copolymers thereof, terpolymers thereof, combinations thereof, and mixtures thereof.

7. The composition of claim 1 wherein the biodegradable, biocompatible thermoplastic polymer comprises at least one polyester.

8. The composition of claim 1 wherein the biodegradable, biocompatible thermoplastic polymer is at least one of a polylactide, a polyglycolide, a polycaprolactone, a copolymer thereof, a terpolymer thereof, or any combination thereof.

9. The composition of claim 1 wherein the biodegradable, biocompatible thermoplastic polymer is a poly (DL-lactide-co-glycolide).

10. The composition of claim 1 wherein the biodegradable, biocompatible thermoplastic polymer is a poly (DL-lactide-co-glycolide) having a carboxy terminal group.

11. The composition of claim 1 wherein the biodegradable, biocompatible thermoplastic polymer is a poly (DL-lactide-co-glycolide) without a carboxy terminal group.

12. The composition of claim 1 wherein the biodegradable, biocompatible thermoplastic polymer is 50/50 poly (DL-lactide-co-glycolide) having a carboxy terminal group.

13. The composition of claim 1 wherein the biodegradable, biocompatible thermoplastic polymer is 75/25 poly (DL-lactide-co-glycolide) without a carboxy terminal group.

14. The composition of claim 1 wherein the biodegradable, biocompatible thermoplastic polymer is present in up to about 80 wt. % of the composition.

15. The composition of claim 1 wherein the biodegradable, biocompatible thermoplastic polymer is present in more than about 10 wt. % of the composition.

16. The composition of claim 1 wherein the biodegradable, biocompatible thermoplastic polymer is present in about 10 wt. % to about 80 wt. % of the composition.

17. The composition of claim 1 wherein the biodegradable, biocompatible thermoplastic polymer is present in about 30 wt. % to about 50 wt. % of the composition.

18. The composition of claim 1 wherein the biodegradable, biocompatible thermoplastic polymer has an average molecular weight of more than about 15,000.

19. The composition of claim 1 wherein the biodegradable, biocompatible thermoplastic polymer has an average molecular weight of up to about 45,000.

20. The composition of claim 1 wherein the biodegradable, biocompatible thermoplastic polymer has an average molecular weight of about 15,000 to about 45,000.

21. The composition of claim 1 wherein the biocompatible organic liquid has a water solubility ranging from completely insoluble in any proportion to completely soluble in all proportions.

22. The composition of claim 1 wherein the biocompatible organic liquid is completely insoluble in water but will diffuse into body fluid.

23. The composition of claim 1 wherein the biocompatible organic liquid is at least partially water-soluble.

24. The composition of claim 1 wherein the biocompatible organic liquid is completely water-soluble.

25. The composition of claim 1 wherein the biocompatible organic liquid is a polar protic liquid.

26. The composition of claim 1 wherein the biocompatible organic liquid is a polar aprotic liquid.

27. The composition of claim 1 wherein the biocompatible organic liquid is a cyclic, aliphatic, linear aliphatic, branched aliphatic or aromatic organic compound, that is liquid at ambient and physiological temperature, and contains at least one functional group selected from the group of alcohols, ketones, ethers, amides, amines, alkylamines, esters, carbonates, sulfoxides, sulfones, and sulfonates.

28. The composition of claim 1 wherein the biocompatible organic liquid is selected from the group of substituted heterocyclic compounds, esters of carbonic acid and alkyl alcohols, alkyl esters of monocarboxylic acids, aryl esters of monocarboxylic acids, aralkyl esters of monocarboxylic acids, alkyl esters of dicarboxylic acids, aryl esters of dicarboxylic acids, aralkyl esters of dicarboxylic acids, alkyl esters of tricarboxylic acids, aryl esters of tricarboxylic acids, aralkyl esters of tricarboxylic acids, alkyl ketones, aryl ketones, aralkyl ketones, alcohols, polyalcohols, alkylamides, dialkylamides, alkylsulfoxides, dialkylsulfoxides, alkylsulfones, dialkylsulfones, lactones, cyclic alkyl amides, cyclic alkyl amines, aromatic amides, aromatic amines, mixtures thereof, and combinations thereof.

29. The composition of claim 1 wherein the biocompatible organic liquid is selected from the group of N-methyl-2-pyrrolidone, 2-pyrrolidone, (C₂-C₈) aliphatic alcohol, glycerol, tetraglycol, glycerol formal, 2,2-dimethyl-1,3-dioxolone-4-methanol, ethyl acetate, ethyl lactate, ethyl butyrate, dibutyl malonate, tributyl citrate, tri-n-hexyl acetylcitrate, diethyl succinate, diethyl glutarate, diethyl malonate, triethyl citrate, triacetin, tributyrin, diethyl carbonate, propylene carbonate, acetone, methyl ethyl ketone, dimethylacetamide, dimethylformamide, caprolactam, dimethyl sulfoxide, dimethyl sulfone, tetrahydrofuran, caprolactam, N,N-diethyl-m-toluamide, 1-dodecylazacycloheptan-2-one, 1,3-dimethyl-3,4,5,6-tetrahydro-2-(1H)-pyrimidinone, benzyl benzoate, and combinations thereof.

30. The composition of claim 1 wherein the biocompatible organic liquid has a molecular weight in the range of about 30 to about 500.

31. The composition of claim 1 wherein the biocompatible organic liquid is N-methyl-2-pyrrolidone, 2-pyrrolidone, N,N-dimethylformamide, dimethyl sulfoxide, propylene carbonate, caprolactam, triacetin, or any combination thereof.

32. The composition of claim 1 wherein the biocompatible organic liquid is N-methyl-2-pyrrolidone.

33. The composition of claim 1 wherein the biocompatible liquid is present in more than about 40 wt. % of the composition.

34. The composition of claim 1 wherein the biocompatible liquid is present in up to about 80 wt. % of the composition.

35. The composition of claim 1 wherein the biocompatible liquid is present in about 50 wt. % to about 70 wt. % of the composition.

36. The composition of claim 1 wherein the biocompatible liquid is dispersible in aqueous medium, water, or body fluid.

37. The composition of claim 1 wherein the cell-cycle dependent biological agent or schedule-dependant biological agent is a compound that blocks, impedes, or otherwise interferes with, cell cycle progression at the G1-phase, G1/S interface, S-phase, G2/M interface, or M-phase of the cell cycle; or is a metabolite or prodrug thereof.

38. The composition of claim 37 wherein the compound is:
an analogue of a uridine nucleoside, an analogue of a thymidine nucleoside, an analogue of a uridine nucleoside, or an analogue of a thymidine nucleoside;
a modulator of a fluoropyrimidine;
a cytidine analogue or a cytidine nucleoside analogue;
a purine analogue or a purine nucleoside analogue;
an antifolate;
an antimetabolite;
an S-phase specific radiotoxin (deoxythymidine analogue);
an inhibitor of an enzyme involved in deoxynucleoside/deoxynucleotide metabolism;
a DNA chain-terminating nucleoside analogue;
an inhibitor of an enzyme that regulates, directly or indirectly, cell cycle progression through the G 1-phase, G1/S interface or S-phase of the cell cycle;
a cytokine, growth factor, anti-angiogenic factor or other protein that inhibits cell cycle progression at the G1-phase or G1/S interface of the cell cycle;
a drug or compound that inhibits cell cycle progression at the G2/M interface, or M-phase of the cell cycle;
a taxane microtubule-targeting drug;
a vinca alkaloid microtubule-targeting drug;
another microtubule-targeting drug;
an inhibitor of serine-threonine kinase, that regulate progression through the G2/M interface or M-phase of the cell cycle; or
a metabolite or prodrug thereof.

39. The composition of claim 38 wherein the analogue of a uridine nucleoside, analogue of a thymidine nucleoside, analogue of a uridine nucleoside, analogue of a thymidine nucleoside, metabolite thereof, or prodrug thereof, is 5-fluorodeoxyuridine (floxuridine, FUDR), 5-Flurouracil (5-FU), a prodrug of 5-FU, bromodeoxyuridine, iododexoyuridine, or a prodrug of halopyrimidine.

40. The composition of claim 39 wherein the prodrug of 5-FU is capecitabine, 5′-deoxy-5-fluorouridine, ftorafur, or flucytosine.

41. The composition of claim 39 wherein the prodrug of halopyrimidine is a polymeric prodrugs of halopyrimidine.

42. The composition of claim 38 wherein the modulator of a fluoropyrimidine is leurovorin, methotrexate, levamisole, acivicin, phosphonacetyl-L-aspartic acid (PALA), brequinar, or 5-ethynyluracil uracil.

43. The composition of claim 38 wherein the a cytidine analogue, cytidine nucleoside analogue, metabolite or prodrug thereof, is cytarabine (Ara-C, cytosine arabinoside), Gemcitabine (2′,2′-difluorodeoxycytidine), 5-azacytidine, or a prodrug of a cytidine analogue.

44. The composition of claim 43 wherein the prodrug of a cytidine analogue is a polymeric prodrug of a cytidine analogue.

45. The composition of claim 38 wherein the purine analogue, purine nucleoside analogue, metabolite thereof or prodrug thereof, is 6-thioguanine, 6-mercaptopurine, azathioprine, adenosine arabinoside (Ara-A), 2′,2′-difluorodeoxyguanosine, deoxycoformycin (pentostatin), cladribine (2-chlorodeoxyadenosine), an inhibitor of adenosine deaminase, or a prodrug of a purine analogue.

46. The composition of claim 38 wherein the prodrug of a purine analogue is a polymeric prodrug of a purine analogue.

47. The composition of claim 38 wherein the antifolate, metabolite thereof, or prodrug thereof, is methotrexate, aminopterin, trimetrexate, edatrexate, N10-propargyl-5,8-dideazafolic acid (CB3717), ZD1694, 5,8-dideazaisofolic acid (IAHQ), 5,10-dideazatetrahydrofolic acid (DDATHF), 5-deazafolic acid (efficient substrate for FPGS), PT523 (N alpha-(4-amino4-deoxypteroyl)-N delta-hemiphthaloyl-L-omithine), 10-ethyl-10-deazaaminopterin (DDATHF, lomatrexol), piritrexim, 10-EDAM, ZD1694, GW1843, PDX (10-propargyl-10-deazaaminopterin), multi-targeted folate, a folate-based inhibitor of thymidylate synthase (TS), a folate-based inhibitor of dihydrofolate reductase (DHFR), a folate-based inhibitor of glycinamide ribonucleotide transformylase (GARTF), an inhibitor of folylpolyglutamate synthetase (FPGS), a folate-based inhibitor of GAR formyl transferase (AICAR transformylase).

48. The composition of claim 47 wherein the multi-targeted folate is LY231514 or permetrexed.

49. The composition of claim 38 wherein the antimetabolite is hydroxyurea or a polyamine.

50. The composition of claim 38 wherein the S-phase specific radiotoxin (deoxythymidine analogue) is [¹²⁵I]-iododeoxyuridine, [¹²³I]-iododeoxyuridine, [¹²⁴I]-iododeoxyuridine, [^{80m}Br]-iododeoxyuridine, [¹³¹I]-iododeoxyuridine, or [²¹¹At]-astatine-deoxyuridine.

51. The composition of claim 38 wherein the inhibitor of an enzyme involved in deoxynucleoside/deoxynucleotide metabolism is an inhibitor of thymidylate synthase (TS), an inhibitor of dihydrofolate reductase (DHFR), an inhibitor of glycinamide ribonucleotide transformylase (GARTF), an inhibitor of folylpolyglutamate synthetase (FPGS), an inhibitor of GAR formyl transferase (AICAR transformylase), an inhibitor of DNA Polymerase (DNA Pol), an inhibitor of ribonucleotide reductase (RNR), an inhibitor of thymidine kinase (TK), or an inhibitor of topoisomerase I enzymes.

52. The composition of claim 51 wherein the inhibitor of DNA Polymerase is Aphidocolin.

53. The composition of claim 51 wherein the inhibitor of topoisomerase I enzymes is camptothecins, irinotecan [CPT-11, camptosar], topotecan, NX-211 [lurtotecan] or rubitecan.

54. The composition of claim 38 wherein the a DNA chain-terminating nucleoside analogue is acyclovir, abacavir, valacyclovir, zidovudine (AZT), didanosine (ddI, dideoxycytidine), zalcitabine (ddC), stavudine D4T), lamivudine (3TC), a 2′3′-dideoxy nucleoside analogue, or a 2′3′-dideoxy nucleoside analogue that terminates DNA synthesis.

55. The composition of claim 38 wherein the inhibitor of an enzyme that regulates, directly or indirectly, cell cycle progression through the G1-phase, G1/S interface or S-phase of the cell cycle is an inhibitor of growth factor receptor tyrosine kinases that regulates progression through the G1-phase, G1/S interface, or S-phase of the cell cycle, an inhibitor of non-receptor tyrosine kinases, an inhibitor of serine-threonine kinases that regulate progression through the G1-phase, G1/S interface or S-phase of the cell cycle, an inhibitor of G-proteins and cGMP phosphodiesterases that positively regulate cell cycle progression at the G1-phase, G1/S interface or S-phase of the cell cycle, a drug that inhibits the induction of immediate early response transcription factors, or a drug that inhibits proteosomes that degrade negative cell cycle regulatory compounds.

56. The composition of claim 55 wherein the inhibitor of growth factor receptor tyrosine kinases that regulates progression through the G1-phase, G1/S interface, or S-phase of the cell cycle is trastusumab, iressa, erbitux, or tarceva.

57. The composition of claim 55 wherein the inhibitor of non-receptor tyrosine kinase is gleevec.

58. The composition of claim 38 wherein the cytokine, growth factor, anti-angiogenic factor or other protein that inhibits cell cycle progression at the G1-phase or G1/S interface of the cell cycle is an interferon, interleukin, somatostatin, a somatostatin analogue, or an anti-angiogenic factor that inhibits cell proliferation of endothelial cells at the G1 or G1/S phases of the cell cycle.

59. The composition of claim 58 wherein the somatostatin or somatostatin analogue is octreotide or sandostatin LAR.

60. The composition of claim 38 wherein the microtubule-targeting drug is taxol, taxotere, epothilones, a taxane derivative, vinca alkaloid, vinblastine, vincristine, vindesine, vinflunine, vinorelbine, vinzolidine, nocadazole, colchicine, estramustine or CP461.

61. The composition of claim 38 wherein the inhibitor of serine-threonine kinase, that regulates progression through the G2/M interface or M-phase of the cell cycle, is an inhibitor of G2/M cyclin-dependent kinase, an inhibitor of M-phase cyclin, or a drug that blocks, impedes, or otherwise interferes with, cell cycle progression at the G2/M interface, or M-phase of the cell cycle.

62. The composition of claim 1 wherein the cell-cycle biological agent, schedule-dependant biological agent, metabolite thereof, pharmaceutically acceptable salt thereof, or prodrug thereof is present in more than about 0.00001 wt. % of the composition.

63. The composition of claim 1 wherein the cell-cycle biological agent, schedule-dependant biological agent, metabolite thereof, pharmaceutically acceptable salt thereof, or prodrug thereof is present in up to about 20 wt. % of the composition.

64. The composition of claim 1 wherein the cell-cycle biological agent, schedule-dependant biological agent, metabolite thereof, pharmaceutically acceptable salt thereof, or prodrug thereof is present in about 0.00001 wt. % to about 10 wt. % of the composition.

65. The composition of claim 1 wherein the human maximum tolerated dose (MTD) of the cell-cycle biological agent, schedule-dependant biological agent, metabolite thereof, or prodrug thereof, present in the flowable composition is less than the human maximum tolerated dose (MTD) of the cell-cycle biological agent, 25 schedule-dependant biological agent, metabolite thereof, or prodrug thereof, present in solution.

66. The composition of claim 1 wherein the human maximum tolerated dose (MTD) of the cell-cycle biological agent, schedule-dependant biological agent, metabolite thereof, or prodrug thereof, present in the flowable composition is at least 50% less than the human maximum tolerated dose (MTD) of the cell-cycle biological agent, schedule-dependant biological agent, metabolite thereof, or prodrug thereof, present in solution.

67. The composition of claim 1 further comprising at least one of:
a release rate modification agent for controlling the rate of release of the cell-cycle biological agent or schedule-dependant biological agent in vivo from an implant matrix;
a pore-forming agent;
a biodegradable, crystallization-controlling agent;
a plasticizer;
a leaching agent;
a penetration enhancer;
an absorption altering agent;
an opacification agent; and
a colorant.

68. The composition of claim 67 wherein the release rate modification agent is selected from the group of an ester of a monocarboxylic acid, an ester of a dicarboxylic acid, an ester of a tricarboxylic acid, a polyhydroxy alcohol, a fatty acid, a triester of glycerol, a sterol, an alcohol, and any combination thereof.

69. The composition of claim 67 wherein the release rate modification agent is selected from the group of 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, propylene glycol, polyethylene glycol, glycerin, sorbitol, triglyceride, epoxidized soybean oil, cholesterol, a (C₆-C₁₂) alkanol, 2-ethoxyethanol, and any combination thereof.

70. The composition of claim 67 wherein the pore-forming agent is a sugar, salt, water-soluble polymer, or water-soluble organic liquid.

71. The composition of claim 67 wherein the biodegradable, crystallization-controlling agent is selected from the group of calcium carbonate, hydroxyapatite, calcium phosphate, calcium apatite, calcium sulfate, calcium bicarbonate, calcium chloride, sodium carbonate, sodium bicarbonate, sodium chloride, calcium stearate, calcium palmitate, sodium stearate, dextran, starch, sodium carboxymethyl cellulose, carboxymethyl cellulose, hydroxyethyl cellulose, hydroxypropyl cellulose, cross-linked sodium carboxymethyl cellulose, poly(vinyl alcohol), glycerol palmitate, glycerol stearate, triethyl citrate, ethyl lactate, poly(ethylene glycol), poly(vinyl pyrrolidone), poly(lactide-co-caprolactone), and combinations thereof.

72. The composition of claim 67 wherein the modifying agent is selected from the group of benzyl benzoate, phthalic esters, benzylphthalates, glycol benzoates, trimellitates, adipates, azelates, sebacates, esters of aliphatic and aromatic di- and tricarboxylic acids, organic phosphates, sesame oil, soybean oil, cotton seed oil, almond oil, sunflower oil, peanut oil, and combinations thereof.

73. The composition of claim 67 wherein the absorption altering agent is selected from the group of propylene glycol, glycerol, urea, diethyl sebecate sodium, lauryl sulfate, sodium lauryl sulfate, sorbitan ethoxylates, oleic acid, pyrrolidone carboxylate esters, N-methylpyrrolidone, N,N-diethyl-m-tolumide, dimethyl sulfoxide, alkyl methyl sulfoxides, and combinations thereof.

74. The composition of claim 67 wherein the rate modification agent is a water insoluble organic substance.

75. The composition of claim 74 wherein the water insoluble organic substance is an ester of a mono-, di- or tricarboxylic acid.

76. The composition of claim 67 wherein the opacification agent comprises barium, iodine, or calcium.

77. The composition of claim 1 wherein the cell-cycle biological agent, schedule-dependant biological agent, metabolite thereof, pharmaceutically acceptable salt thereof, or prodrug thereof is incorporated into a particulate or encapsulated controlled-release component.

78. The composition of claim 77 wherein the particulate controlled-release component comprises a conjugate in which the cell-cycle biological agent, schedule-dependant biological agent, metabolite thereof, pharmaceutically acceptable salt thereof, or prodrug thereof is covalently bonded to a carrier molecule.

79. The composition of claim 77 wherein the particulate controlled-release component is a microstructure selected from the group of a microcapsule, a nanoparticle, a cyclodextrin, a liposome, and a micelle.

80. The composition of claim 77 wherein the particulate controlled-release component is a microstructure of less than about 500 microns.

81. The composition of claim 77 wherein the particulate controlled-release component is a macrostructure selected from the group of a fiber, film, rod, disc and cylinder.

82. The composition of claim 77 wherein the particulate controlled release-component is a macrostructure of at least about 500 microns.

83. The composition of claim 1 that is capable of forming a solid microporous matrix, the matrix being a core surrounded by a skin and the core containing pores of diameters from about 1 to about 1000 microns.

84. The composition of claim 83 wherein the skin contains pores of smaller diameters than those of the core pores such that the skin is functionally non-porous in comparison with the core.

85. The composition of claim 1 having a volume of more than about 0.001 mL.

86. The composition of claim 1 having a volume of up to about 20.0 mL.

87. The composition of claim 1 having a volume of about 0.01 mL to about 10.0 mL.

88. The composition of claim 1 that is formulated for administration less than about once per week.

89. The composition of claim 1 that is formulated for administration more than about once per year.

90. The composition of claim 1 that is formulated for administration about once per week to about once per year.

91. The composition of claim 1 that delivers the cell-cycle biological agent, schedule-dependant biological agent, metabolite thereof, pharmaceutically acceptable salt thereof, or prodrug thereof to mammalian tissue at a dosage of about 1 picogram/kilogram/day to about 1 milligram/kilogram/day.

92. The composition of claim 91 wherein the delivery is systemic delivery.

93. The composition of claim 91 wherein the delivery is local delivery.

94. The composition of claim 91 wherein the dosage is delivered locally for a period of time of up to about 1 year.

95. The composition of claim 91 wherein the dosage is delivered locally for a period of time of up to about 1 month.

96. The composition of claim 91 wherein the dosage is delivered locally for a period of time of up to about 1 week.

97. The composition of claim 91 wherein the dosage is delivered locally for a period of time of more than about 1 day.

98. The composition of claim 1 further comprising a second chemotherapeutic agent.

99. The composition of claim 98 wherein the second chemotherapeutic agent acts at various stages of the cell cycle.

100. The composition of claim 99 wherein the second chemotherapeutic agent is an antracycline, a DNA intercalator, an alkylating agent, a hormonal agent, a chemoprevention agent, a metabolite thereof, or a prodrug thereof.

101. The composition of claim 100 wherein the antracycline is doxorubicin, daunorubicin, epirubicin, idarubicin, or mitoxantrone.

102. The composition of claim 100 wherein the DNA intercalator is actinomycin C, actinomycin D, actinomycin B, a podophyllotoxin, or an epipodophyllatoxin.

103. The composition of claim 102 wherein the epipodophyllatoxin is etoposide, teniposide, or ctoposide.

104. The composition of claim 100 wherein the alkylating agent is mechlorethamine, melphalan, cyclophosphamide, chlorambucil, ifosfamide, carmustine, lomustine, busulfan, dacarbazine, cisplatin, carboplatin, oxaliplatin, iproplatin, or tetraplatin.

105. The composition of claim 100 wherein the hormonal agent is an antiestrogen/estrogen antagonist, an LHRH agonist or antagonist, an aromatase inhibitor, or an antiandrogen.

106. The composition of claim 105 wherein the LHRH agonist or antagonist is leuprolide acetate, goserelin, or abarelix.

107. The composition of claim 100 wherein the chemoprevention agent is an NSAID or cis-retinoid.

108. A method of treating cancer in a mammal, the method comprising administering to a mammal in need of such treatment an effective amount of a flowable composition comprising:
(a) a biodegradable, biocompatible thermoplastic polymer that is at least substantially insoluble in aqueous medium, water or body fluid;
(b) a cell-cycle dependent biological agent, a schedule-dependent biological agent, a metabolite thereof, a pharmaceutically acceptable salt thereof, or a prodrug thereof; and
(c) a biocompatible organic liquid at standard temperature and pressure, in which the thermoplastic polymer is soluble.

109. The method of claim 108 wherein the mammal is a human.

110. The method of claim 108 wherein the cancer is a solid tumor.

111. The method of claim 108 wherein the cancer is a solid tumor located in the breast, lung, thyroid, lymph node, genitourinary system, kidney, ureter, bladder, ovary, testis, prostate, musculoskeletal system, bone, skeletal muscle, bone marrow, gastrointestinal tract, stomach, esophagus, small bowel, colon, rectum, pancreas, liver, smooth muscle, central or peripheral nervous system, brain, spinal cord, nerves, head, neck, ear, eye, nasopharynx, oropharynx, salivary gland, cardiovascular system, oral cavity, tongue, larynx, hypopharynx, soft tissues, skin, cervix, anus, retina, or heart.

112. The method of claim 108 wherein the flowable composition is administered in multiple locations of the mammal.

113. A method of blocking, impeding, or otherwise interfering with cell cycle progression at the G1-phase, G1/S interphase, S-phase, G2/M interface or M-phase of the cell cycle, the method comprising administering to a mammal in need of such blocking, impeding, or interfering, an effective amount of a flowable composition comprising:
(a) a biodegradable, biocompatible thermoplastic polymer that is at least substantially insoluble in aqueous medium, water or body fluid;
(b) a cell-cycle dependent biological agent, a schedule-dependent biological agent, a metabolite thereof, a pharmaceutically acceptable salt thereof, or a prodrug thereof; and
(c) a biocompatible organic liquid at standard temperature and pressure, in which the thermoplastic polymer is soluble.

114. An implant comprising:
(a) a biodegradable, biocompatible thermoplastic polymer that is at least substantially insoluble in aqueous medium, water or body fluid;
(b) a cell-cycle dependent biological agent, a schedule-dependent biological agent, a metabolite thereof, a pharmaceutically acceptable salt thereof, or a prodrug thereof; and
(c) a biocompatible organic liquid at standard temperature and pressure, in which the thermoplastic polymer is soluble;
wherein the implant has a solid or gelatinous microporous matrix, the matrix being a core surrounded by a skin and wherein the implant is surrounded by body tissue.

115. The implant of claim 114 that has fully coagulated.

116. The implant of claim 114 that has solidified

117. The implant of claim 114 wherein the amount of biocompatible organic liquid decreases over time.

118. The implant of claim 114 wherein the core contains pores of diameters from about 1 to about 1000 microns.

119. The implant of claim 114 wherein the skin contains pores of smaller diameters than those of the core pores.

120. The implant of claim 114 wherein the skin pores are a size such that the skin is functionally non-porous in comparison with the core.

121. An implant comprising:
(a) a biodegradable, biocompatible thermoplastic polymer that is at least substantially insoluble in aqueous medium, water or body fluid; and
(b) a cell-cycle dependent biological agent, a schedule-dependent biological agent, a metabolite thereof, a pharmaceutically acceptable salt thereof, or a prodrug thereof;
wherein the implant has a solid or gelatinous microporous matrix, the matrix being a core surrounded by a skin and wherein the implant is surrounded by body tissue.

122. The implant of claim 121 wherein the core contains pores of diameters from about 1 to about 1000 microns.

123. The implant of claim 121 wherein the skin contains pores of smaller diameters than those of the core pores.

124. The implant of claim 121 wherein the skin pores are a size such that the skin is functionally non-porous in comparison with the core.

125. A method of forming an implant in situ within a living body, the method comprising:
(a) injecting a flowable composition within the body of a patient, the composition comprising:
(i) a biodegradable, biocompatible thermoplastic polymer that is at least substantially insoluble in aqueous medium, water or body fluid;
(ii) a cell-cycle dependent biological agent, a schedule-dependent biological agent, a metabolite thereof, a pharmaceutically acceptable salt thereof, or a prodrug thereof; and
(iii) a biocompatible organic liquid at standard temperature and pressure, in which the thermoplastic polymer is soluble; and
(b) allowing the biocompatible organic liquid to dissipate to produce a solid biodegradable implant.

126. A pharmaceutical kit suitable for in situ formation of a biodegradable implant in a body, the kit comprising:
(a) a first container comprising a flowable composition, the composition comprising:
(i) a biodegradable, biocompatible thermoplastic polymer that is at least substantially insoluble in aqueous medium, water or body fluid; and
(ii) a biocompatible organic liquid at standard temperature and pressure, in which the thermoplastic polymer is soluble;
(b) a second container comprising a cell-cycle dependent biological agent, a schedule-dependent biological agent, a metabolite thereof, a pharmaceutically acceptable salt thereof, or a prodrug thereof.

127. The kit of claim 126 wherein the first container is a syringe.

128. The kit of claim 126 wherein the first container comprises a catheter.

129. The kit of claim 126 wherein the second container is a syringe.

130. The kit of claim 126 wherein the second container comprises a catheter.

131. The kit of claim 126 wherein the first container is a syringe, the second container is a syringe, and both syringes are configured to directly connect to each other.

132. The kit of claim 126 further comprising instructions.

133. The composition of any of claims 1 for use in medical therapy or diagnosis.

134. A use of the composition of any of claims 1 for the manufacture of a medicament for treating cancer.
